(19) United States
(12) Patent Application Publication
(10) Pub. No.: US 2007/0224287 A1
(43) Pub. Date: Sep. 27, 2007

(54) Methods of Modulating Inflammatory Reactions by Modulating Xanthine Oxidoreductase Activity

(76) Inventors: Richard M. Wright, Denver, CO (US); John E. Repine, Denver, CO (US)

Correspondence Address: MORGAN LEWIS & BOCKIUS LLP, 1111 PENNSYLVANIA AVENUE NW, WASHINGTON, DC, 20004, US

(21) Appl. No.: 10/573,354

(22) PCT Filed: Sep. 27, 2004
(86) PCT No.: PCT/US04/31478

§ 371 Date: May 10, 2007

Related U.S. Application Data
(60) Provisional application No. 60/505,922, filed on Sep. 26, 2003.

(51) Int. Cl. A61K 31/519; A61K 33/24; C12N 9/02

(52) U.S. Cl. 424/617; 435/189; 514/262.1

(57) ABSTRACT

Evidence is presented that inflammation and injury involves activation of xanthine oxidoreductase (XOR) in the newly recruited mononuclear phagocytes (MNP). XOR has been shown to be increased predominantly in the MNP that increase rapidly in the lungs of rats that develop acute lung injury (ALI) following intratracheal cytokine insufflation. XOR was recovered from the MNP largely converted to its oxygen radical generating, reversible O-form, and alveolar MNP exhibited increased oxidative stress as evidenced by increased nitrotyrosine staining. Cytokine insufflation also increased alveolar cell apoptosis. A functional role for XOR in cytokine induced inflammation was demonstrated. Tungsten and allopurinol decreased MNP XOR induction, nitrotyrosine staining, inflammatory cell infiltration, and alveolar cell apoptosis. Transfer of control or allopurinol treated MNP into rat lungs and confirmed a specific role for MNP XOR in promoting lung inflammation. These data indicate that XOR can contribute to lung inflammation by its expression and conversion in a highly mobile inflammatory cell population.

### CROSS-REFERENCE TO RELATED APPLICATIONS

This application is related to U.S. Provisional Application No. 60/505,922, filed Sep. 26, 2003, which is herein incorporated by reference in its entirety.

### STATEMENT REGARDING FEDERALLY SPONSORED RESEARCH OR DEVELOPMENT

This invention was made with U.S. government support in part by grants from the National Institutes of Health (HL52509 and HL45582), The Department of Defense (BC980149), and the Robert and Helen Kleberg Foundation. The U.S. government has certain rights in the invention.

### FIELD OF THE INVENTION

The present invention generally pertains to a method of modulating, including methods of treating and/or preventing, inflammatory diseases involving leukocytes and their precursor cells in a subject in need thereof The present invention also pertains to methods of modulating xanthine oxidoreductase (XOR) activity in leukocytes and leukocyte precursor cells in vivo or in vitro.

### BACKGROUND OF THE INVENTION

Xanthine oxidoreductase (XOR) contributes to diverse inflammatory diseases of humans and in animal models of inflammatory disease. The specific XOR inhibitors, allopurinol and tungsten, have been used to infer the contribution of XOR in human and in animal models of inflammatory disease.

Heart disease. XOR contributes significantly to diverse heart diseases. The cardio-protective effect of allopurinol in patients with chronic heart failure (CHF) (7-9), cardiomyopathy (10), or in patients undergoing heart surgery (11,12) identifies a heretofore unrecognized contribution of XOR to these diseases. These observations are striking since the normal heart is a poor source of XOR (13). While the mechanism by which XOR mediates heart disease is unknown, inflammation is now recognized to contribute substantially to each of these disorders (7-12).

Diabetes and pancreatic inflammation. Allopurinol treatment has been protective of vascular endothelial function in Type 1 and Type 2 diabetes in humans (14,15), and XOR was markedly elevated in several organs in experimentally induced diabetes in rats (16). In streptozotocin induced diabetes in mice, for example, allopurinol normalized superoxide generation (17). Furthermore, allopurinol blocked pancreatic fibrosis in rats treated with the inflammatory inducer, diethyldithiocarbamate (18), and blocked evidence of pancreatitis and vascular injury in L-arginine induced pancreatic inflammation in rats (19).

Liver and gut inflammatory injury. In humans with Crohn's disease, epithelial damage was strongly associated with expression of XOR (20), and in mice, allopurinol treatment markedly reduced I/R induced colonic leukocyte-chemokine expression and leukocyte accumulation (21,22). Leukocyte-chemokine expression and leukocyte accumulation in an endotoxemic model of liver injury (23) were likewise blocked with allopurinol treatment. Furthermore, treatment with allopurinol blocked the progression of experimentally induced acute gastric mucosal lesions in rats and decreased markers of leukocyte infiltration in the gut (24). Inflammatory lung disorders are among the most common lethal diseases of humans and become the third or fourth leading cause of death with advancing age (1-3). The complex immunological mechanisms mediating inflammatory lung disorders involve cells of both innate and adaptive immunity (1-5). For example, acute lung injury (ALI), the acute respiratory distress syndrome (ARDS), and lung ischemia/reperfusion injury (I/R) are acute inflammatory disorders typified by a large influx of leukocytes, including polymorphonuclear phagocytes (PMN or neutrophils) and mononuclear phagocytes (MNP). Chronic obstructive pulmonary disease (COPD), tuberculosis, and sarcoidosis are chronic inflammatory disorders that typically reveal the participation of both MNP cells and T-helper1 (Th1) cells of adaptive immunity (1-6).

Inflammation of the eye. Very recent data have shown the marked immunomodulatory effect of allopurinol on inflammatory cell accumulation in clinical and experimental uveitis (25-27). Allopurinol was as effective as steroid treatment in its capacity to reduce PMN accumulation in the eye even after the onset of disease, and with its minimal side effects, allopurinol offers great promise as an alternative therapy for inflammatory diseases of the eye. No mechanism has been postulated for the immunomodulation produced by allopurinol in inflammatory eye disease.

Lung and vascular inflammatory injury. XOR and its substrate, hypoxanthine, were elevated in the lungs and blood of patients with ALI (28,29), and up regulation of XOR in many organs, including the lung, is a molecular signature of systemic inflammation induced by sepsis (30), a common cause of ALI. Furthermore, XOR is up regulated in the bronchoalveolar lavage fluid of patients with COPD (31,32), and allopurinol treatment reduced both XOR and evidence of XOR derived oxidative stress in COPD (33,34). Feeding animals on tungsten or allopurinol diets also reduced inflammatory lung injury produced by hemorrhage (35-38) and reduced vascular permeability that was induced experimentally by I/R (38,39). Our work demonstrated that allopurinol and tungsten both attenuated inflammation and markers of lung injury in a rat model of cytokine induced lung inflammation (40). Allopurinol treatment was also found to induce remission in patients with sarcoidosis (41-45), a chronic inflammatory injury of both the skin and the lung. These results are noteworthy for the capacity of allopurinol to cause even fulminate sarcoid lesions to fully regress. Allopurinol treatment was likewise found to reduce endothelial injury in the lungs (and gut) of heavy smokers and in rats subjected to heavy smoking conditions (46-48). Finally, evidence of vascular injury induced in mice by hemorrhagic shock (49) or LPS injection (50) were also dramatically attenuated by prior inhibition of XOR with allopurinol.

Transplantation. Oxidative stress occurs in ischemia-reperfusion injury and may also contribute to tissue rejection damage. It is thought to play a major role in activation of endothelium, the primary target in allograft rejection. Several cellular sources of reactive oxygen species (ROS) are known, including the mitochondria, oxidant enzymes in peroxisomes, cytoplasmic enzymes like xanthine oxidoreductase (XOR), and the plasma membrane-bound NAD(P)H oxidase complexes. ROS at low concentrations function as mediators of intracellular signaling cascades; however, ROS at high concentration may result in oxidative modification of lipids, proteins, and DNA. ROS can also act as chemoattractants for infiltrating leukocytes, which may worsen tissue injury because of their ability to release proteases and inflammatory cytokines. Therefore, ROS, including superoxide anion, hydrogen peroxide, hydroxyl radical, and singlet oxygen, have been implicated in the pathophysiology of a wide variety of human diseases including ischemia-reperfusion injury.

Xanthine oxidoreductase (XOR) catalyses the final reactions of purine catabolism, fostering the hydroxylation of hypoxanthine to xanthine and xanthine to uric acid, and may account for cell damage by producing reactive oxygen metabolites in cells reoxygenated after hypoxia. In normal tissues, XOR exists mostly as xanthine dehydrogenase (XDH) using as the electron acceptor; it can be converted into an oxidase (XO) in vivo and in vitro. In contrast with XDH, XO uses molecular oxygen as the electron acceptor and produces substantial amounts of superoxide and hydrogen peroxide under appropriate conditions. Recent studies have shown that XOR expression and activity is induced by inflammation and by cytokines where it can promote inflammation as a product of the invading mononuclear phagocyte, and inhibition of XOR can be protective in a wide variety of inflammatory conditions.

In principal, ROS produced predominantly by increased XOR activity could also modulate the extent of tissue rejection. Pretreatment with tungsten (WfC12), which inhibits XOR activity by replacing the molybdenum ion within the enzyme and thereby inactivating its catalytic site, could alleviate the damage of grafts after transplantation by a decrease in XOR activity and consequently a reduction of ROS generation. This hypothesis was recently tested (Sun et al., Role of Xanthine Oxidoreductase in Experimental Acute Renal-Allograft Rejection. Transplantation 77: 1683-1692, 2004). The authors confirmed the role of XOR in experimental graft rejection and observed the following: “Generation of ROS was enhanced, being 10fold higher in renal allografts versus control kidneys at day 9 (P<0.01); this was associated with histologic signs of acute rejection. Oxygen radicals were generated to a significant degree by enhanced XOR activity, which increased more than 10-fold in renal allografts at day 9 posttransplantation; XOR protein in glomeruli and tubulointerstitium was also elevated in allografts. In addition, NADPH oxidase activity increased significantly in allografts. The activity of antioxidant enzymes tended to decrease. Tungsten treatment resulted in a pronounced reduction of XOR activity and ROS production, without any effect on NADPH-oxidase activity; mononuclear cell infiltration and rejection signs were significantly ameliorated at day 9 post-transplantation by selective inhibition of XOR. A major part of ROS generation in acute rejection was contributed by XOR. ROS are not only associated with but also contribute to acute allograft rejection because inhibition of XOR alleviated rejection phenomena.”

The contribution of XOR to inflammatory lung disorders is not well understood.

However, XOR could mediate inflammation as a source of reactive oxygen species (ROS) or reactive nitrogen species (RNS). While XOR is uniquely responsible for the synthesis of uric acid in the course of purine degradation, it can also generate ROS with high efficiency. Both H₂O₂ and O₂^{-.} are produced from XOR following conversion of D-form XOR (xanthine dehydrogenase, XDH) to O-form XOR (xanthine oxidase, XO) (51).

Conversion from D-form to O-form can be mediated in vitro following proteolysis or thiol oxidation (52,53), but it remains a poorly characterized process in vivo. We established the contribution of XOR to cytokine induced lung inflammation and oxidative stress using tungsten and allopurinol inhibition (40). Both of these inhibitors decreased lung inflammation, MNP nitrotyrosine staining, and alveolar cell apoptosis. Allopurinol is a highly specific XOR inhibitor that acts noncompetitively following its conversion into oxypurinol (alloxanthine) which then inhibits the molybdenum center of XOR by tight, but reversible, binding (54). In our experiments, rats were fed allopurinol at a dose of 50 mg/Kg for seven days, a regimen well known to inhibit XOR in vivo (35,40). While high concentrations of allopurinol may theoretically have ROS scavenging capability in vivo (55), this results primarily from quenching hydroxyl radical and not O₂⁻ (56).

Furthermore, allopurinol, even at relatively high doses, fails to scavenge ROS generated from the NADPH oxidase, confirming that allopurinol is not, per se, a significant scavenger of O₂^{-.} in vitro or in vivo (57-59). Tungsten feeding is another selective method for inhibiting XOR. Tungsten displaces molybdenum from the molybdopterin cofactor necessary for the activity of XOR and other MoCo enzymes, thereby precluding substrate reduction of the enzyme (60). The similar effect achieved by treatment with either of these distinct and differently acting inhibitors strongly suggest that XOR activity contributed to the inflammatory responses described above.

Although XOR is largely recognized to contribute to inflammatory and vascular injury as a source of ROS, recent evidence indicates that XOR can also convert nitrates into nitric oxide (NO) under hypoxic conditions (61-63), contributing to the formation of peroxynitrite and perhaps other RNS (64). This kinetically efficient reaction contributes to hypoxic myocardial NO synthesis at levels equivalent to those of nitric oxide synthase (65), and may contribute significantly to the formation of RNS in human COPD (34). Thus, XOR could play a complex role in inflammatory disorders as a source of both ROS and RNS.

MNPs play essential and multiple roles in inflammation. MNP are derived from differentiation of hematopoietic bone marrow stem cells, and the complex life-cycle of the MNP suggests several ways in which the MNP affect the course of inflammation. The role played by the MNPs in inflammatory lung disorders is not well understood. However, resident alveolar macrophages (AM) are potent sources of pro-inflammatory cytokines such as TNF-α, IL-1, IL-8, IFN-γ or ROS (1-3) that can promote inflammation. Resident, mature AM comprise close to 100% of the cells found in the BALF of resting, untreated rats, and when detected lymphocytes and PMNs comprise well below 1% of the total inflammatory cell population (40). Thus the resting lung may be considered to harbor an essentially pure population of mature AM. The resting mature AM responds to many activating signals including products of bacterial pathogens like the lipopolysaccharide (LPS) and cytokines such as IL-1 or IFN-γ (98-101). Upon activation, AM secrete cytokines involved in the orchestration of the inflammatory response, activate the respiratory burst NADPH oxidase, and release ROS. For example, IL-1, IL-6, IL-8, and TNF-α are all released in response to LPS (98,99). The response of alveolar macrophages to IFN-γ is complex. IFN-γ is a principle activating factor in vivo and is responsible for activation of the NADPH oxidase and general reprogramming of macrophage gene transcription and subsequent cytokine release (100,101). IFN-γ signal transduction in the macrophage operates through a system of protein relays that involves activation of the JAK/STAT pathway and NF-κB activation (102). Thus, the AM comprise cells highly responsive to pathogens and cytokines, not only as phagocytic cells, but as cells capable of calling up and amplifying the inflammatory process. As phagocytic cells, the resident AM also play vital roles in the resolution of inflammation through removal of apoptotic cells (103).

The recruitment, infiltration, and differentiation of monocytes into macrophages is a critical feature of lung inflammation that underlies the role played by the MNPs. Following the induction of inflammation, monocytes are recruited to the lung primarily, but not exclusively, in response to the chemokines MCP-1 and MCP-2 (104-106). Once established in the air space, monocytes rapidly differentiate into macrophages by a complex process that has been initiated from the earliest moments of transendothelial migration (107-110). Recognition of the differentiation program has evolved over the last 15 years and considerable data have now been marshaled that comprise a solid framework for understanding monocyte to macrophage differentiation (111-113). In essence the program of differentiation begins in the bone marrow with the differentiation of hematopoietic stem cells into monocytes, which are best understood as committed but still undifferentiated cells with the potential to become several different end stage cells depending upon the tissue in which they localize (114). For example monocytes differentiate into osteoclasts in bone, Kupfer cells in the liver, macrophages in the peritoneum or lung, etc. Differentiation of circulating monocytes into macrophages occurs once these cells have left the vascular compartment. Cytokine driven differentiation sequentially activates specific subsets of transcription factors that mediate differentiation (111,112). Not all of these signals are fully understood, however, factors committing precursor cells to the monocytic lineage include STAT1, GATA-1, PU-1, AML-1, and C/EBP whose DNA binding signatures can all be found on the upstream regulatory DNA of mouse, rat, and human XOR. Differentiation of the committed monocyte into a macrophage is mediated by the factors C/EBP, NF-Y, AML-1, STAT1, STAT5 and others, whose signatures are also key features of the XOR regulatory DNA. Cytokines promoting differentiation include GM-CSF, M-CSF, IL-6, NF-γ, and TGF-β, although signals derived from adhesion molecule ligation and bone morphogenetic proteins (BMP2-7) also contribute to MNP differentiation (115-117).

Cytokines and chemokines expressed by MNP orchestrate the inflammatory process. MNP can participate in the autocrine and paracrine synthesis of proinflammatory cytokines and chemokines resulting in amplification of the inflammatory process. For example, human monocytes in culture or established human monocytic cell lines express and secrete IFN-γ, RANTES, MIP-1α, MCP-1, MCP-2, and IL-8(107-109,118-123). Autocrine stimulation of mouse macrophages by IFN-γ results in stimulated expression of TNF-α, MCP-1, MIP-1aα and IL-8 (121-123). Thus, while PMN and MNP chemokines can be derived from endothelial or epithelial cells (124,125), they can also be derived from the inflammatory MNP themselves. Recently, newly infiltrating MNP were found to promote PMN immigration, recruitment, and infiltration following intratracheal instillation of LPS in mice (126,127) or cytokine in rats (40). Although the mechanism for MNP induced PMN recruitment was not defined in these studies, MNP are sources of both peptide (IL-8, Cinc) and lipid PMN chemotactic factors following activation (128-130). Thus, inflammatory activation of the MNP can greatly expand the inflammatory cell population and promote injury.

Intracellular ROS derived from XOR could modulate the function of MNP during inflammation. While many different sources of ROS can be defined in the MNP, both XOR and the NADPH oxidase have been functionally linked to inflammation in humans and in rodent models of inflammation. The different compartments into which ROS are released by each of these enzymes can be anticipated to differentiate their respective contributions to inflammation.

XOR as an intracellular source of ROS. The expression of both XOR and the NADPH oxidase by MNP would appear to provide redundant sources of O2-., however, this redundancy may be more apparent than real. First, there is now overwhelming support for the oxidation of low density lipoprotein (LDL) by MNP NADPH oxidase which contributes to the inflammatory response of atherosclerosis (131). Cell specific knockout of NADPH oxidase precludes LDL oxidation by activated monocytes and precludes O2-. synthesis (132). It is important that the MNP NADPH oxidase is assembled in a polarized sense on the MNP plasma membrane, with O2-. being generated “outside” the cell and therefore released to an extracellular environment where it can participate in LDL oxidation (133). Routine assay of NADPH oxidase derived O2-. involves exposing plated cells to cytochrome c, whose reduction is monitored spectrophotometrically in the absence of cell lysis (132). Even during phagocytosis, NADPH oxidase derived O2-. will be released to a membrane bound subcellular compartment, the phagolysosome. On the other hand, XOR is a soluble cytoplasmic enzyme whose ROS will necessarily be released to the cytoplasm where they are anticipated to undergo rapid reactivity with cytoplasmic components (51-54). Thus, XOR and the NADPH oxidase may provide the MNP with functionally distinct and compartmentalized sources of O2-.

As described above, XOR is largely recognized to contribute toinflammatory injury as a source of ROS. The capacity for XOR to convert nitrates into NO during inflammation (62, 63) will contributing to the formation of peroxynitrite and perhaps other RNS (64). This kinetically efficient reaction contributes to hypoxic myocardial NO synthesis at levels equivalent to those of nitric oxide synthase (65), and contributes significantly to the formation of RNS in human COPD (34). Once again, as a cytoplasmic enzyme, RNS generated by MNP XOR would also be generated initially in the intracellular compartment where they would be immediately available to modulate RNS sensitive intracellular signaling. Thus, XOR could play a complex role in inflammation as a source of urate, ROS, or RNS.

ROS signaling in the MNP. The capacity for NADPH oxidase generated extracellular O₂^{-.} to affect redox signaling in the MNP has been exhaustively reviewed (134,135). Several points emerge from these analyses that suggest possible ways in which intracellular XOR derived ROS could modulate MNP function as well. First, the exceptional reactivity of O₂^{-.} confers upon it a spatial restriction for its reactivity. The restricted location of action would provide specificity in signal transduction. For example, extracellular O₂^{-.} released from the NADPH oxidase must first undergo dismutation to H₂O₂ prior to entering the MNP, while O₂^{-.} released from XOR to the intracellular compartment would be immediately available for reactivity. Second, intracellular steady state O₂^{-.} concentration is estimated to be 10⁻¹¹ M, and even substantial shift in concentration could be achieved by relatively low levels of XOR activity. Third, under limiting substrate conditions, the preferred XOR derived ROS is O₂^{-.} and this would distinguish the affects of the NADPH oxidase from those of XOR. Finally, intracellular redox signaling in the MNP is complex (134), involving activation of redox sensitive transcription factors (NF-κB, AP-1, SP-1), ERK/MEK kinases, tyrosine phosphatases, and lipid mediators (134). Furthermore, reactivity of each of these down stream mediators will be affected by the immediate cytokine environment whose signaling is transduced by many of the same pathways.

The profile of cytokines and chemokines expressed by MNPs can be sensitive to intracellular ROS. Analysis of IL-8 gene expression in MNP indicates that it is activated in response to ROS via NF-κB and AP-1 mechanisms (120-123,128) and may, therefore, be sensitive to intracellular ROS. Given the broad reactivity of the pathways sensitive to intracellular redox signaling, it is presently impossible to anticipate specific targets of XOR derived ROS.

Proliferation of MNP in response to intracellular ROS. In addition to an effect on MNP cytokine or chemokine expression, intracellular ROS can affect the growth or apoptosis of cells (136,137). In particular, granulocyte-macrophage colony stimulating factor (GM-CSF) promotes the growth and differentiation of granulocytes and MNP from hematopoietic stem cells (138,139). GM-CSF has been also recognized to promote a proliferative phase of the MNP (140) that depends upon the generation of ROS (141-143). GM-CSF, acting either through tyrosine MAP kinases or the JAK2/STAT5 signaling complex, stimulates ROS generation (142) leading to an increase in MNP mitosis and cell proliferation (141,143), and ROS appear to be critical for entry of MNP into the G1-S phase of the cell cycle thereby inducing mitosis (141). Interestingly, these observations were presaged 20 years ago by the demonstration that increased MNP mitosis was one mechanism mediating expansion of the MNP population in a chronic inflammatory disorder (144). These observations reveal the key role played by intracellular ROS in the expansion of the MNP population and suggest an additional important point at which XOR derived intracellular ROS may also regulate MNP function.

Cytokines elevated in the lungs of patients with inflammatory lung disorders could promote XOR dependent ROS generation. Cytokines and other factors that are increased in the bronchoalveolar lavage fluid (BALF) of ALI patients and in animal models of inflammatory lung injury include (in part) IL-1, IFN-65 , TNF-α, IL-4, IL-6, IL-8, IL-10, TGF-β, GM-CSF, M-CSF, MCP-1, and MCP-2(145-147). While the balance between pro- and anti- inflammatory cytokines and chemokines appears to determine the progression of ALI and its outcome (145-147), this balance may itself be critical for the induction and expression of XOR, and cytokine activation of XOR may be a necessary prerequisite for its involvement in lung inflammation.

Cytokines that are elevated in inflammatory lung disorders can increase lung XOR expression and subsequent ROS production. In particular, IL-1 is increased in inflammatory lung disorders and may contribute to inflammation by inducing the expression of inflammatory chemokines (IL-8, MCP-1) and adhesion molecules important for migration of inflammatory cells into the air space (ICAM-1) (145-148). Intratracheal insufflation of IL-1 promotes inflammatory cell accumulation and acute lung leak in rats (149,150) and induces expression of inflammatory chemokines (IL-8, MCP-1) and adhesion molecules (ICAM-1) in cultured lung epithelial cells in vitro (148). Importantly, XOR steady state RNA and enzyme levels are regulated in several cultured cells by IL-1 through a cycloheximide and an actinomycin-D inhibitable mechanism (78-80). IL-1 alone induces XOR by approximately 4 fold in cultured endothelial and epithelial cells (79,80). IFN-y is also increased in the BALF of ALI patients (145-147), induces inflammatory cell recruitment in vivo (151,152), and promotes apoptosis in cultured lung alveolar epithelial cells in vitro (153). IFN-γ also induces XOR steady state RNA, protein, and enzyme activity by mechanisms potentially synergistic with IL-1 in cultured cells in vitro and in vivo in mice (78-80,154,155). IL-6 has been reported to induce XOR in cultured epithelial cells (78-80), and synergy between IL-1 and IL-6 increases this induction to 10 fold in vitro. In a cytokine model of ALI, alveolar MNP XOR levels were dramatically elevated, and the MNP XOR recovered was largely converted into O-form (40).

Transcriptional activation of XOR gene expression by inflammatory cytokines may underlie its contribution to inflammatory lung injury. Most analyses of XOR gene expression have been conducted with cultured epithelial cells, endothelial cells, or fibroblasts where XOR was induced by proinflammatory cytokines, LPS, or hypoxia (78-81). Our work with mammary gland XOR demonstrates that XOR gene expression and MRNA content are induced over 80% in vivo by lactation (81,156), and in vitro, XOR was induced in cultured mammary epithelial cells by prolactin and cortisol (81,157). While XOR can be regulated by post-transcriptional mechanisms such as phosphorylation and mRNA turnover (71), regulation at the transcriptional level is a primary component in the regulation of XOR gene expression. In cultured cells, cytokine or hormone activation of XOR was blocked by actinomycin D (78-80) as was activation in response to hypoxic growth (71,80,158).

Efforts to understand XOR transcriptional regulation have been undertaken in several laboratories. Upstream regulatory DNA from the XOR genetic locus has been fused to luciferase reporter genes and transient expression studied in cultured cells. Deletion analysis of XOR upsteam DNA for human and rat XOR produced a basic understanding of the XOR promoter and regulatory DNA. First, human and rat XOR reveal complex architecture in the upstream DNA that maintains XOR under a state of repression that, presumably, must be released before XOR can be activated (159). Second, a basal promoter can be defined from approximately +1 to −100 nt that is sufficient for activation in all cells examined so far (89,159,160). Third, a region of transcription initiation at −59 nt was identified in several cells that corresponds to the site selected in vivo (91,159,160). Fourth, a domain within the first 42 nt of the rat XOR exon 1 is required for proper transcription site selection in vitro and this region binds a transcription factor of the C/EBP class (91). Fifth, a fractional site upstream of the transcription initiation site has been identified that is essential for activation of the human XOR gene, and this site binds the transcription factor NF-Y (160). Importantly, the functional NF-Y site observed in the human XOR promoter is conserved in nearly the identical position in human, rat, and mouse sequences. Sixth, many binding sites within the first 200 nt upstream of the translational start site can bind members of the C/EBP class (91,160), and other binding sites found in this region include an E-box ARE site, and sites for YY1 and USF1 (89-91,159,160). In mammary epithelial cells, XOR transcriptional activation by prolactin and dexamethasone was blocked by inhibitors of the JAK2/STAT5 and glucocorticoid pathways (157) and by inhibitors of the MEK-1/2 and ERK-1/2 pathways (156).

Many of the signaling pathways that activate XOR in endothelial and epithelial cells are shared with the MNP. Research into the mechanism of inflammatory disorders for much of the preceding several decades has focused on the induction of inflammation and the role played by endothelial cells and endothelial cell XOR in this process. Most of our present knowledge about XOR regulation has been obtained from analyses of cultured endothelial and epithelial cells. However, recognition that the inflammatory leukocytes, particularly MNPs, express high levels of XOR, and that inhibition of XOR is protective in many inflammatory disorders, is important because XOR derived intracellular ROS could exert many effects on the life of the leukocytes and thereby potentiate inflammation many different levels.

Therefore, a need continues to exist for the development of a method that will be effective to modulate inflammatory reactions involving leukocytes and their precursor cells. In addition, a need also continues to exist for the development of compounds which regulate XOR activity in vivo and in vitro in leukocytes and leukocyte precursor cells.

### SUMMARY OF THE INVENTION

The invention includes a method of modulating inflammatory reactions involving leukocytes and leukocyte precursor cells in a subject comprising contacting a subject in need of said modulating with an amount of a xanthine oxidoreductase (XOR) inhibitor effective to modulate said inflammatory reaction involving leukocytes and leukocyte precursor cells in said subject. The invention is also directed to a method of modulating XOR activity in leukocytes and leukocyte precursor cells comprising contacting said leukocytes and leukocyte precursor cells with an agent which modulates the expression, synthesis, degradation, secretion, release, half-life, conversion or catalysis of XOR in leukocytes and leukocytes precursor cells thereby modulating XOR activity. Also included in the invention is a method of modulating inflammatory reactions involving leukocytes and leukocyte precursor cells in a subject comprising contacting said subject in need of said modulating with an amount of an agent which is effective to modulate the expression, synthesis, degradation, secretion, release, half-life, conversion or catalysis of XOR in leukocytes and leukocyte precursor cells thereby modulating said inflammatory reactions.

The invention is further directed to a method of modulating cytokine-induced inflammation in a subject comprising a) removing leukocytes and leukocyte precursor cells from said subject to form a population of leukocytes and leukocyte precursor cells; b) contacting said population of leukocytes and leukocyte precursor cells formed in part a) with an effective amount of one or more agents to obtain a treated cell population, wherein said one or more agents are effective to modulate the expression, synthesis, degradation, secretion, release, half-life, conversion or catalysis of XOR in leukocytes and leukocyte precursor cells; and, c) administering said treated cell population of part b) to said subject; wherein said administered cell population modulates said cytokine-induced inflammation in said subject.

### BRIEF DESCRIPTION OF THE DRAWINGS

FIGS. 1A-E. Lung XOR induction following cytokine insufflation. Total lung and liver XOR activity was quantitated in the presence of NAD⁺ in soluble desalted protein extracts from rats that were insufflated 24h before with saline, IL-1, IFN-γ or IL-1 and IFN-γ. Data show the mean and standard error for six rats in each group. IL-1 and IFN-γ insufflated rats had increased (**, p<0.02) lung (A), but not (p>0.05) liver (B), XOR activity compared to rats insufflated with saline, IL-1 (*, p<0.05), or IFN-γp>0.05). (C) Western immunoblot analysis of lung XOR from cytokine insufflated rats. Antibody to rat β-actin was used to control for gel loading. Gels were scanned with a Perkin-Elmer phosphoimager for semi-quantitation. Data are shown for two rats in each group. These data demonstrate that lungs from rats insufflated with IL-1 and IFN-γ had increased XOR immunoreactive protein compared to rats insufflated with saline, IL-1 or IFN-γ. (D) The percentage of O-form XOR was quantitated in soluble protein extracts of cytokine insufflated rat lungs. Data show the mean and standard error for six rats in each group and are expressed as the percentage of the total XOR activity. Rats insufflated with IL-1 and IFN-γ had increased (*, p<0.05) lung O-form XOR activity compared to rats insufflated with saline, IL-1 or IFN-γ. (E) Soluble protein extracts were reduced in the presence of 5 mM dithiothreitol (DTT) for one hour at 37° C. after which they were re-chromatographed on Sephadex G25 and re-assayed as above. O-form XOR activity in the soluble protein extracts from all insufflated rats was comparably reduced by DTT. Data show the mean and standard error for six rats in each group.

FIGS. 2A-F. Induction of XOR in lung inflammatory cells. (A) BALF from rats insufflated 24 h before with IL-1 and IFN-γ had increased (**, p<0.02) numbers of inflammatory cells compared to rats insufflated with saline, IL-1, or IFN-γ alone. Data show the mean and standard error for eight rats in each group. (B) Lung tissue slices were mounted on glass slides, H&E stained, examined under bright-field microscopy, and digitized using the Roper Scientific Digital Imaging program (Roper Scientific, Trenton, N.J.). Lungs from rats insufflated with IL-1 and IFN-λ, 24 h before had increased numbers of airway and perivascular inflammatory cells compared to rats insufflated with saline, IL-1 or IFN-γ. (C) Immunofluorescent detection of XOR immunoreactive protein in paraffin embedded lung tissue sections using rhodamine conjugated anti-XOR antisera (XOR, panels A,C,E,G,I). Lung architecture was delineated by staining with FITC conjugated wheat germ agglutinin (WGA, panels B,D,F,H,J). Higher magnifications (Panels I and J) depict increased XOR immunofluorescence primarily associated with the inflammatory cells in the alveolar space of rats insufflated with IL-1 and IFN-γ. (D) Western immunoblot analysis of XOR protein in cells recovered from the BALF of cytokine insufflated rats. Antibody to rat β-actin was used to control for gel loading. Data are shown for two rats in each group. XOR protein was elevated in the BALF cells following IL-1 and IFN-γ insufflation compared to the response observed following saline, IL-1 or IFN-γ insufflation. (E,F) Quantitation of D-form and O-form XOR in BALF cells recovered from cytokine insufflated rat lungs. D-form XOR activity (e) was increased in the BALF cells from rats insufflated with IL-1 and IFN-γ compared to rats insufflated with saline (p<0.02), IL-1 or IFN-γ alone. XOR was recovered in predominantly O-form in BALF cells from rats insufflated with IL-1 and IFN-γ (F). Data show the mean and standard error for 6 rats in each group.

FIGS. 3A-E. Induction of XOR in the infiltrating and differentiating MNP. (A) Differential analysis of inflammatory cells recovered sequentially over an 18 day time course from the lungs of rats insufflated with IL-1 and IFN-γ. (B) FACS analysis of cells recovered from the BALF of IL-1 and IFN-γ insufflated rats. As described in detail in the text, BALF cells were stained with the monocyte and neutrophil marker, CD11 b, or the alveolar macrophage marker, ED1, and were then analyzed by FACS. Ten thousand cells were analysed for each staining reaction and representative scatter diagrams are depicted for each time point. For each fluorescence curve, cell numbers are plotted on the ordinate and fluorescence intensity on the abscissa. Filled curves represent net fluorescence while the black line indicates fluorescence due to the non-specific antisera. In the scatter diagrams, forward scatter is plotted along the abscissa and side scatter along the ordinate. These data demonstrate the rapid influx of neutrophils and monocytes and the maturation of monocytes in the lung following cytokine insufflation. (C) Western immunoblot analysis of Percoll-gradient purified neutrophils and MNP recovered from the BALF of rats 24 hours following insufflation with IL-1 and IFN-γ shows the expression of XOR in MNP but not neutrophils (D, E) Quantitation of D-form (D) and O-form (E) XOR in adherent MNP recovered over a 24 hour time course from rats insufflated with IL-1 and IFN-γ. Data show the mean and standard error for three rats at each time point. The inset depicts a western immunoblot showing increased XOR activity for cells recovered at each time point. Cells recovered from the BALF of rats insufflated with saline alone 8 or 24 hours previously (sham treated controls) showed only negligible XOR activity and immunoreactivity (not shown) similar to that observed at the 0 time control.

FIGS. 4A-H. Effect of tungsten or allopurinol feeding on lung and MNP XOR, BALF cell numbers, MNP nitrotyrosine staining, and alveolar cell apoptosis. Lung XOR activity (A), MNP XOR activity (B), BALF cell number (C), lung histologic abnormalities (D), MNP nitrotyrosine staining (E), and alveolar cell apoptosis (F,G,H) were all increased (**, p<0.02) following insufflation of IL-1 and IFN-γ compared to saline insufflation. In contrast, rats fed tungsten or allopurinol diets prior to IL-1 and IFN-γ insufflation had decreased (* p<0.05 or ** p<0.02) lung XOR activity (A), MNP XOR activity (B), BALF cell numbers (C), lung histologic abnormalities (D), MNP nitrotyrosine staining (E), alveolar cell TUNEL stain (F,G), and alveolar cell caspase-3 activation (H) compared to rats fed a control diet and then insufflated with IL-1 and IFN-γ. Nitrotyrosine immunofluorescence staining (E) was conducted on lung tissue sections 24 hours following IL-1 and IFN-γ insufflation. Nitrotyrosine immuno-fluorescence is shown in red and tissue architecture in green and blue. Allopurinol and tungsten inhibition of MNP nitrotyrosine immunofluorescence and the effect of nitrotyrosine prebinding (NT pre-binding) are also shown (E). These data demonstrate increased oxidative stress in the lungs of rats insufflated with IL-1 and IFN-γ and that immunofluorescence in the alveolar located MNP was attenuated by prior inhibition of XOR. In Figure (G), panels 1 and 2 show higher magnification fields of fluorescence TUNEL staining associated with alveolar cells. Each data point in the quantitative TUNEL assay represents the counting of at least 8,000 nuclei derived from tandem serial sections of clearly identifiable alveolar cells. Data were acquired for each tissue slice by counting 200 nuclei per field and 20 fields per slide in parallel transects across each slide. These data demonstrate the quantitative increase in TUNEL stain following cytokine insufflation and the subsequent decrease in TUNEL stain in rats previously fed allopurinol or tungsten diets. Active caspase-3 (H) was quantitated in a similar fashion by counting the percentage of activated caspase-3 positive cells per high field view in 20 fields per tissue for two rats in each group. Activated caspase-3 was increased in the lungs of rats insufflated with IL-1 and IFN-γ compared to rats insufflated with saline (**, p<0.02) or tungsten (*, p<0.05) or allopurinol (**, p<0.02) fed rats insufflated with IL-1 and IFN-γs. Since allopurinol dissociates from XOR during preparation of the protein extracts, allopurinol inhibition of XOR was not assayed (n.d.) in Figures (A) and (B).

FIGS. 5A-C. MNP XOR augments lung inflammation in vivo. A lung inflammatory response was induced in rats with IL-1 and IFN-γ insufflation. After 8 hours, cells from BALF were recovered from each rat in PBS, pooled, and quantitated. Cells were then divided into two equivalent fractions and exposed to 1 mM KOH or 1 mM KOH with 150 μM allopurinol in vitro. The neutral pH of the PBS and cell mixture was unchanged by adding 1 mM KOH. After 15 min, the cells were washed and resuspended in PBS. Subsequently, 2×10⁶ of control or allopurinol treated cells were insufflated into control rats. 24 hours after cell insufflation, BALF cells were recovered from these rats, Wright's stained and quantitated. Insufflation of untreated BALF cells increased the recovery of neutrophil 24 h later (A) compared to insufflation of allopurinol treated BALF cells (B). Quantitation of neutrophils from both groups is shown in (C). The numbers of neutrophils recoverable in BALF from rats insufflated with control cells is significantly increased (* p<0.05) compared to the number of neutrophils recovered following insufflation of allopurinol treated cells. Data are the mean and standard error of 6 rats in each group.

FIGS. 6A-D. XOR Was Induced in the Adherent, Interstitial, and Parenchymal MNP by Cytokine Insufflation. High levels of O-form XOR were demonstrated in the MNP recovered by lavage following IL-1 and IFN-γ induced lung inflammation in rats (Wright et al., 2004), and these cells exhibited excess protein nitration and promoted allopurinol inhibitable alveolar cell apoptosis and neutrophil recruitment. Since IL-1 and IFN-γ could promote MNP adhesion to the respiratory epithelium, adjacent paraffin embedded lung tissue specimens were examined for XOR immunoreactive protein and the macrophage specific markers, ED1 and ED2. Twenty four hours following cytokine insufflation XOR immunoreactive protein was detected in MNP in several lung compartments. FIG. 6 shows that XOR colocalized with the MNP markers ED1 and ED2 in the AM and in the MNP found in the perivascular tissue space, the interstitial alveolar compartment, and in MNP adherent to the alveolar epithelium. These observations are consistent with the involvement of XOR in COPD, which is now well recognized, and other interstitial lung diseases.

### DETAILED DESCRIPTION OF THE PREFERRED EMBODIMENTS

Definitions

As is generally the case in biotechnology and chemistry, the description of the present invention has required the use of a number of terms of art. Although it is not practical to do so exhaustively, definitions for some of these terms are provided here for ease of reference. Unless defined otherwise, all technical and scientific terms used herein have the same meaning as commonly understood by one of ordinary skill in the art to which this invention belongs. Definitions for other terms also appear elsewhere herein. However, the definitions provided here and elsewhere herein should always be considered in determining the intended scope and meaning of the defined terms. Although any methods and materials similar or equivalent to those described herein can be used in the practice of the present invention, the preferred methods and materials are described.

As used herein, the term “agent” refers to any compound which is pharmacologically active and/or modulates XOR activity in leukocytes in vitro or in vivo. The terms “compound” and “agent” are used interchangeably except where specifically indicated. Where a difference is intended, such will be made clear. Accordingly, the agents of the present invention are those which modulate at least one function or characteristic of an XOR protein. The ability of an agent to modulate such a fumction can be demonstrated in a binding assay (e.g., ligand binding or agonist binding), a signalling assay, a catalytic assay and/or cellular response function, for example, stimulation of chemotaxis, exocytosis or inflammatory mediator release by leukocytes and leukocyte precursor cells.

The agent may modulate XOR bioactivity. Agents may act on any of a variety of different levels to affect XOR activity or bioactivity, including regulation of XOR gene expression at the promoter region (for example, transcription factors), regulation of MRNA splicing mechanisms, stabilization of MRNA, phosphorylation of proteins for translation, translation, conversion of XDH to XOR, conversion of XDH to XOR, secretion of XOR, release of XOR, XOR half-life, XOR stability, XOR degradation, and XOR catalytic ability. The invention envisions modulating XOR activity at every level in leukocytes and their precursor cells.

Inhibitor. As used herein, the term “inhibitor” refers to a compound which decreases, or completely inhibits, a chemical reaction. Here, the term inhibitor refers to compounds which competitively and/or non-competitively inhibit XOR. Inhibitors include those compounds which inhibit the molybdenum center of XOR by binding thereto. Inhibitors also include those compounds which displace molybdenum from the molybdenum cofractor necessary for the activity of XOR.

Inhibition. As used herein, the term “inhibition” refers to a decrease in XOR activity which results from contacting the enzyme with the inhibitor.

Modulate. As used herein, the term “modulate” refers to a change, such as a decrease or increase in an existing cellular condition, such as XOR activity or inflammation, or amount of an enzyme, such as the amount of XDH or XOR.

Mononuclear phagocytes. As used herein, the term “mononuclear phagocytes” [MNPs] refers to cells derived from differentiation of hematopoietic bone marrow stem cells which affect the course of inflammation.

Pharmaceutically acceptable. As used herein, the term “pharmaceutically acceptable” means that the agent is compatible with other ingredients of the formulation or composition and not injurious to the patient. Several pharmaceutically acceptable ingredients are known in the art and official publications such as THE UNITED STATES PHARMACOPEIA describe the analytical criteria used to assess the pharmaceutical acceptability of numerous ingredients of interest. As used herein, the phrase “pharmaceutically acceptable salt(s),” means salt(s) formed from an acid and an XOR inhibitor. Preferred salts include, but are not limited to, sulfate, citrate, acetate, oxalate, chloride, bromide, iodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, and p-toluenesulfonate. Many other salts are known to those of skill in the art and are envisioned for use herein with the inhibitors of the present invention.

Preventing. As used herein, “prevention” or “preventing” refers to a reduction of the risk of acquiring inflammation. In one embodiment, an inhibitor of the invention is administered as a preventative measure to a subject. According to this embodiment, the subject can have a genetic or a non-genetic predisposition to inflammation. Accordingly, the agents of the invention can be used for the treatment of one manifestation of inflammation and prevention of another. See, for example, U.S. Pat. No. 6,583,309.

Subject. As used herein, the term “subject” broadly refers to any animal that is to be treated with the agents and by the methods disclosed herein. As used herein, the term means an animal, including, but not limited, to a cow, monkey, horse, sheep, pig, chicken, turkey, quail, cat, dog, mouse, rat, rabbit, and guinea pig. In a preferred embodiment, the animal is a mammal, and, in a highly preferred embodiment, is a human. In a preferred embodiment, the term includes humans in need of, or desiring modulation of, XOR activity in leukocytes in vitro or in vivo. In another preferred embodiment, the term refers to humans in need of, or desiring modulation of inflammation.

Treating. As used herein, the term “treatment” or “treating” refers to a modulation of, or a decrease in inflammation, or at least one discernible symptom thereof such as XOR activity, for example. In another embodiment, “treatment” or “treating” refers to a decrease of at least one measurable physical parameter, not necessarily discernible by the subject. In yet another embodiment, “treatment” or “treating” refers to inhibiting the progression of inflammation, either physically, such as, for example, stabilization of a discernible symptom, or physiologically, for example, the stabilization of a physical parameter such as XOR activity, or both. In yet another embodiment, “treatment” or “treating” refers to delaying the onset of inflammation. See, for example, U.S. Pat. No. 6,583,309.

Vehicle. As used herein, the term “vehicle” refers to a diluent, adjuvant, excipient, or carrier with which a compound of the invention is administered.

The invention includes a method of modulating inflammatory reactions involving leukocytes and leukocyte precursor cells in a subject comprising contacting a subject in need of said modulating with an amount of a xanthine oxidoreductase (XOR) inhibitor effective to modulate said inflammatory reaction involving leukocytes and leukocyte precursor cells in said subject. In one embodiment, the leukocytes and leukocyte precursor cells are selected from the group consisting of mononuclear phagocytes, neutrophils and eosinophils. In another embodiment, the inflammatory reaction is a disease selected from the group consisting of chronic heart failure, cardiomyopathy, diabetes, pancreatic inflammation, liver inflammation, Crohn's disease, uveitis, acute lung injury, COPD, sarcoidosis, granulomatous lung inflammation (GLI), acute lymphoblastic leukemia (ALL), ischemia reperfusion injury, hemorrhagic shock and renal transplant rejection. In a preferred embodiment, the inflammatory disease is granulomatous lung inflammation. In a different preferred embodiment, the inflammatory disease is acute or chronic lung injury. In another embodiment, the oxidoreductase inhibitor is selected from the group consisting of allopurinol, oxypurinol, tungsten, amflutizole and (−)BOF-4272 ([(−)-8-(3-methoxy-4-phenylsulfinylphenyl) pyrazolo (1,5-alpha)-1,3,5-triazine-4-monohydrate]).

The invention is also directed to a method of modulating XOR activity in leukocytes and leukocyte precursor cells comprising contacting said leukocytes and leukocyte precursor cells with an agent which modulates the expression, synthesis, degradation, secretion, release, half-life, conversion or catalysis of XOR in leukocytes and leukocytes precursor cells thereby modulating XOR activity. In one embodiment, the leukocytes and leukocyte precursor cells are selected from the group consisting of mononuclear phagocytes, neutrophils and eosinophils. In a preferred embodiment, the contacting occurs in vitro. In another preferred embodiment, the contacting occurs in vivo. In a different embodiment, the leukocytes and leuckocyte precursor cells are involved in inflammatory reactions. In one embodiment, the inflammatory reaction is an inflammatory disease selected from the group consisting of chronic heart failure, cardiomyopathy, diabetes, pancreatic inflammation, liver inflammation, Crohn's disease, uveitis, acute lung injury, COPD, sarcoidosis, granulomatous lung inflammation (GLI), acute lymphoblastic leukemia (ALL), ischemia reperfusion injury, hemorrhagic shock and renal transplant rejection. In one preferred embodiment, the inflammatory disease is granulomatous lung inflammation. In another preferred embodiment, the inflammatory disease is acute lung injury. In a different embodiment, the agent is selected from the group consisting of allopurinol, oxypurinol, tungsten, amflutizole and (−)BOF-4272.

Also included in the invention is a method of modulating inflammatory reactions involving leukocytes and leukocyte precursor cells in a subject comprising contacting said subject in need of said modulating with an amount of an agent which is effective to modulate the expression, synthesis, degradation, secretion, release, half-life, conversion or catalysis of XOR in leukocytes and leukocyte precursor cells thereby modulating said inflammatory reactions. In one embodiment, the leukocytes and leukocyte precursor cells are selected from the group consisting of mononuclear phagocytes, neutrophils and eosinophils. In a different embodiment, the inflammatory reaction is an inflammatory disease selected from the group consisting of chronic heart failure, cardiomyopathy, diabetes, pancreatic inflammation, liver inflammation, Crohn's disease, uveitis, acute lung injury, COPD, sarcoidosis, granulomatous lung inflammation (GLI), acute lymphoblastic leukemia (ALL), ischemia reperfusion injury, hemorrhagic shock and renal transplant rejection. In a preferred embodiment, the inflammatory disease is granulomatous lung inflammation. In another preferred embodiment, the inflammatory disease is acute lung injury. In yet another embodiment, the agent is selected from the group consisting of allopurinol, oxypurinol, tungsten, amflutizole and (−)BOF-4272.

The invention is further directed to a method of modulating cytokine-induced inflammation in a subject comprising a) removing leukocytes and leukocyte precursor cells from said subject to form a population of leukocytes and leukocyte precursor cells; b) contacting said population of leukocytes and leukocyte precursor cells formed in part a) with an effective amount of one or more agents to obtain a treated cell population, wherein said one or more agents are effective to modulate the expression, synthesis, degradation, secretion, release, half-life, conversion or catalysis of XOR in leukocytes and leukocyte precursor cells; and, c) administering said treated cell population of part b) to said subject; wherein said administered cell population modulates said cytokine-induced inflammation in said subject. In one embodiment, the leukocytes and leukocyte precursor cells are selected from the group consisting of mononuclear phagocytes, neutrophils and eosinophils. In a different embodiment, the agent is an inhibitor. In a preferred embodiment, the inhibitor is selected from the group consisting of allopurinol, oxypurinol, tungsten, amflutizole and (−)BOF-4272.

Inflammatory Reaction. The cardinal signs of an inflammatory reaction are redness (rubor), heat (calor), pain (dolor), swelling (edema)and loss of fumction (fiinctio laesa). Inflammation can be produced by infectious (bacteria, viral, fingal, etc.) and non-infectious insults, such as heat, cold, radiant energy, electrical, chemical or trauma. Inflammation may be acute or chronic, localized or systemic. See, for example, Stanley L. Robbins. The Pathologic Basis of Disease, WB Saunders Company Philadelphia, London, and Toronto, pp 57-105,1974 and V. Kumar, R S Cotran, and Robbins S. L., Saunders 7th edition. BASIC PATHOLOGY. Chapter 2 by R. N. Mitchell and R. S. Cotran. Acute and Chronic Inflammation. Pp 33-59, 2003.

Agents

Pharmaceutically acceptable vehicles. When administered to a subject, an agent, including inhibitors, of the invention is preferably administered as a component of a composition that comprises a pharmaceutically acceptable vehicle. The present compositions, which comprise a compound of the invention, may be administered orally. The compositions of the invention may also be administered by any other convenient route, for example, by jinfsion or bolus injection, by absorption through epithelial or mucocutaneous linings (e.g., oral mucosa, rectal, and intestinal mucosa, etc.) and may be administered together with another biologically active agent. Administration can be systemic or local. Various delivery systems are known, for example, encapsulation in liposomes, microparticles, microcapsules, capsules, etc., and can be used to administer the compounds of the invention. Other delivery systems known in the art are envisioned in use of the present invention.

Acceptable pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a subject, the pharmaceutically acceptable vehicles are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or buffering agents. See, for example, U.S. Pat. No. 6,583,309.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see, for example, U.S. Pat. No. 5,698,155). Other examples of suitable pharmaceutical vehicles are described in Remington's Pharmaceutical Sciences, Alfonso R. Gennaro ed., Mack Publishing Co. Easton, Pa., 19th ed., 1995, incorporated herein by reference in its entirety. See, particularly pages 1447-1676 and for example, U.S. Pat. No. 6,583,309.

In a preferred embodiment, the compounds of the invention are formulated in accordance with routine procedures as a pharmaceutical composition adapted for oral administration to human beings. Compositions for oral delivery may be in the form of tablets, lozenges, aqueous or oily suspensions, granules, powders, emulsions, capsules, syrups, or elixirs, for example. Orally administered compositions may contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compositions. In these devices, fluid from the environment surrounding the capsule is imbibed by the driving compound, which swells to displace the agent or agent composition through an aperture. These devices can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade. Typically, compositions for intravenous administration comprise sterile isotonic aqueous buffer. Where necessary, the compositions may also include a solubilizing agent.

Methods of Administration

In certain embodiments, an agent of the invention is administered to a subject preferably a human, as a preventative measure against inflammation. Methods of administration include but are not limited to intradermal, intramuscular, intraperitoneal, intravenous, subcutaneous, intranasal, epidural, oral, sublingual, intranasal, intravaginal, transdermal, rectally, by inhalation, or topically, to the skin. The mode of administration is left to the discretion of the practitioner. In most instances, administration will result in the release of a compound of the invention into the bloodstream.

Dosage forms

In dosage forms, the agent can be provided as a powder by freeze drying, spray drying, or the like. Alternatively, it is possible to incorporate the agent into solutions, tablets, granules, dragees, capsules, suspensions, emulsions, ampules, injections, and the like. In certain embodiments, the present compositions can comprise one or more compounds of the invention.

Pulmonary administration can also be employed, for example, by use of an inhaler or nebulizer, and formulation with an aerosolizing agent, or via perfusion in a fluorocarbon or synthetic pulmonary surfactant. In certain embodiments, the compounds of the invention can be formulated as a suppository, with traditional binders and vehicles such as triglycerides.

In another embodiment, the compounds of the invention can be delivered in a vesicle, in particular a liposome. In yet another embodiment, the compounds of the invention can be delivered in a controlled release system such as a pump for example. In another embodiment, polymeric materials can be used. In yet another embodiment, a controlled-release system can be placed in proximity of a target of a compound of the invention, e.g., a particular RNA, thus requiring only a fraction of the systemic dose.

In another embodiment, the compounds of the invention can be formulated for intravenous administration. Compositions for intravenous administration may optionally include a local anesthetic such as lignocaine to lessen pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compounds of the invention are to be administered by infusion, they can be dispensed, for example, with an infusion bottle containing sterile pharmaceutical grade water or saline. Where the compounds of the invention are administered by injection, an ampoule of sterile water for injection or saline can be provided so that the ingredients may be mixed prior to administration.

The amount of a compound of the invention that will be effective in the treatment of a particular type of inflammatory disease or disorder and will depend on the nature of the disorder or condition, and can be determined by standard clinical techniques. In addition, in vitro or in vivo assays may optionally be employed to help identify optimal dosage ranges. The precise dose to be employed will also depend on the route of administration, and the seriousness of the disease or disorder, and should be decided according to the judgment of the practitioner and each patient's circumstances. However, suitable dosage ranges for oral administration are generally about 0.001 milligram to about 200 milligrams of a compound of the invention or a pharmaceutically acceptable salt thereof per kilogram body weight per day. In specific preferred embodiments of the invention, the oral dose is about 0.01 milligram to about 100 milligrams per kilogram body weight per day, more preferably about 0.1 milligram to about 75 milligrams per kilogram body weight per day, more preferably about 0.5 milligram to 5 milligrams per kilogram body weight per day. The dosage amounts described herein refer to total amounts administered; that is, if more than one compound of the invention is administered, or if a compound of the invention is administered with a therapeutic agent, then the preferred dosages correspond to the total amount administered. Oral compositions preferably contain about 10% to about 95% active ingredient by weight.

Suitable dosage ranges for intravenous (i.v.) administration are about 0.01 milligram to about 100 milligrams per kilogram body weight per day, about 0.1 milligram to about 35 milligrams per kilogram body weight per day, and about 1 milligram to about milligrams per kilogram body weight per day. Suitable dosage ranges for intranasal administration are generally about 0.01 pg/kg body weight per day to about 1 mg/kg body weight per day. Suppositories generally contain about 0.01 milligram to about 50 milligrams of a compound of the invention per kilogram body weight per day and comprise active ingredient in the range of about 0.5% to about 10% by weight.

Recommended dosages for intradermal, intramuscular, intraperitoneal, subcutaneous, epidural, sublingual, intracerebral, intravaginal, transdermal administration or administration by inhalation are in the range of about 0.001 milligram to about 200 milligrams per kilogram of body weight per day. Suitable doses for topical administration are in the range of about 0.001 milligram to about 1 milligram, depending on the area of administration. Effective doses may be extrapolated from dose-response curves derived from in vitro or animal model test systems. Such animal models and systems are well known in the art. See, for example, U.S. Pat. No. 6,583,309.

The compounds of the invention are preferably assayed in vitro and in vivo, for the desired therapeutic or prophylactic activity, prior to use in humans. For example, in vitro assays can be used to determine whether it is preferable to administer a compound of the invention alone or in combination with another compound of the invention and/or a therapeutic agent. Animal model systems can be used to demonstrate safety and efficacy. Other methods will be known to the skilled artisan and are within the scope of the invention.

Combination Therapy

In certain embodiments of the present invention, a compound of the invention can be used in combination therapy with at least one other therapeutic agent. The compound of the invention and the therapeutic agent can act additively or, more preferably, synergistically. In a preferred embodiment, a composition comprising a compound of the invention is administered concurrently with the administration of another therapeutic agent, which can be part of the same composition as or in a different composition from that comprising the compound of the invention. In another embodiment, a composition comprising a compound of the invention is administered prior or subsequent to administration of another therapeutic agent. As many of the disorders for which the compounds of the invention are useful in treating are chronic, in one embodiment combination therapy involves alternating between administering a composition comprising a compound of the invention and a composition comprising another therapeutic agent, for example, to minimize the toxicity associated with a particular drug. The duration of administration of the compound of the invention or therapeutic agent can be, e.g., one month, three months, six months, a year, or for more extended periods. In certain embodiments, when a compound of the invention is administered concurrently with another therapeutic agent that potentially produces adverse side effects including, but not limited to, toxicity, the therapeutic agent can advantageously be administered at a dose that falls below the threshold at which the adverse side is elicited. See, for example, U.S. Pat. No. 6,583,309.

Agents to be screened in the practice of the invention include, but are not limited to, compounds that are products of rational drug design, such as small molecule inhibitors, natural products and compounds having defined, undefined, or partially defined activity. An agent can be a protein-based compound, a carbohydrate-based compound, a lipid-based compound, a nucleic acid-based compound, a natural organic compound, a synthetically derived organic compound, an anti-idiotypic antibody and/or catalytic antibody, and fragments thereof An agent can be obtained, for example, from libraries of natural (See, for example, U.S. Pat. No. 6,589,573) or synthetic compounds, in particular from chemical or combinatorial libraries (i.e., libraries of compounds that differ in sequence or size but that have the same building blocks; see for example, U.S. Pat. Nos. 5,010,175 and 5,266,684) or by rational drug design. Many therapeutic agents are known in the art. See, for example, Remington: The Science and Practice of Pharmacy, 1995, Mack Publishing Co., Easton, Pa.

The invention also provides pharmaceutical packs or kits comprising one or more vessels containing a compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains more than one compound of the invention. In another embodiment, the kit comprises a compound of the invention and additionally one or more compounds having therapeutic activity.

Once agents which modulate XOR activity are identified, the agents are further studied both in in vitro and in vivo systems. Such systems are known in the art and are described herein.

ROS derived from the expression and conversion of XOR in the differentiating MNP potentiates the inflammatory response. Analysis of IL-8 gene expression in MNP indicates that it is activated in response to ROS via NF-κB and AP-1 mechanisms (120-123,128) and may, therefore, be sensitive to intracellular ROS. Given the broad reactivity of the pathways sensitive to intracellular redox signaling, it is presently impossible to anticipate specific targets of XOR derived ROS. However, in the broadest terms, intracellular ROS derived from MNP XOR could promote inflammation through the oxidative activation of pro-inflammatory cytokines or chemokines.

Acute lung injury (ALI) is a highly fatal inflammatory disorder characterized by increased alveolar cytokine expression, neutrophil and monocyte recruitment into the lung, macrophage activation, oxidative stress, alveolar cell apoptosis, and lung edema (1,2). Bronchoalveolar lavage fluid (BALF) from ALI patients contains increased concentrations of numerous cytokines and chemokines including IL-1, IFN-γ IL-6, IL-8, TNF-α, IL-10, TGF-β, and MCP-1 (3-5). While the contribution of individual cytokines to the pathophysiology of ALI is not understood, intratracheal insufflation of IL-1 in rats produces lung inflammation with many characteristics typical of ALI. For example, in rats, IL-1 insufflation increased lung edema, BALF protein levels, lung neutrophil recruitment, and oxidative stress (6,7). In cultured lung epithelial cells, IL-1 stimulated expression of inflammatory chemokines (IL-8, MCP-1) and adhesion molecules (ICAM-1) (8) suggesting its potential role in inflammatory cell recruitment. Likewise, insufflation of IFN-γ (9), or its induced expression as a Clara cell transgene (10), also promoted inflammation and macrophage activation in the lung. Therefore, IFN-γ may also contribute to many of the events that arise during lung inflammation including the stimulation of inflammatory cell recruitment (11,12) and the induction of alveolar epithelial cell apoptosis (13). While neutrophils are widely recognized as possible mediators of ALI pathophysiology (14,15), alveolar macrophages also appear to contribute to ALI as sources of proinflammatory cytokines (IL-1 ), chemokines (IL-8, MCP-1), and reactive oxygen species (ROS) (1,2). The recruitment, infiltration, and differentiation of monocytes into macrophages are important steps in the life cycle of the mononuclear phagocytes (MNP) (16-18) that may also impact ALI. Oxidative stress is a common feature of ALI whose contribution to pathogenesis is not well understood (19-22), and while ROS can be derived from many sources during inflammation, xanthine oxidoreductase (XOR) emerged as a possible source because it and its substrate, hypoxanthine, are elevated in the blood and lung lavage of patients with ALI (23-25). Furthermore, feeding animals tungsten or allopurinol, XOR inhibiting diets, reduced ALI induced by hemorrhage (26-28) and vascular permeability induced by ischemia/reperfusion (29). XOR generates ROS with high efficiency following proteolytic or oxidative conversion of D-form XOR (xanthine dehydrogenase, XDH) to O-form XOR (xanthine oxidase, XO). While proteolytic conversion of D-form to O-form has been well studied in vitro (30,31), XOR conversion by thiol oxidation may be a key biological mediator because it is both reversible and potentially subject to regulation. XOR thiol oxidation can be reversed by incubation with reducing agents, like dithiothreitol, to produce D-form XOR (32), and rat liver O-form XOR content has been directly linked to reduced glutathione status (33). O-form XOR is an efficient source of the superoxide anion (O₂^{-.}) (34,35) that can promote protein tyrosine nitration by reacting with nitric oxide (NO) to form peroxynitrite (ONOO), the nitrating species (36,37). Protein nitrotyrosine is a relatively stable modification that provides in vivo evidence of the concomitant presence of NO and O₂^{-.} (38,39).

Alveolar epithelial cell apoptosis has been recognized in the lungs of patients with ALI and in animal models that develop ALI (1,2), can be induced by oxidative stress, and may be responsible for loss of alveolar epithelial function (2). Unlike necrosis, apoptotic cell death involves the ordered activation of effector caspases, such as caspase-3, and the activation of endonucleases that generate DNA nicks that are routinely assayed by fluorescence TUNEL stain (40).

The potential relationship between inflammation and XOR activation was reinforced by the observation that pro-inflammatory cytokines, IL-1 and IFN-γ, induced s XOR in cultured epithelial cells (41-44) while intraperitoneal injection of IFN-γ induced XOR in the lung (45).

The following examples are illustrative of preferred embodiments of the invention and are not to be construed as limiting the invention thereto.

### EXAMPLES

### Example 1

In the present work, we hypothesized that insulation of IL-1 and IFN-γ would induce lung XOR activity and contribute to lung inflammation. Our data not only supported this premise, but revealed XOR induction in the differentiating MNP that increased rapidly in the alveoli following cytokine insufflation. Importantly, MNP XOR induction, MNP oxidative stress, lung inflammation, and alveolar cell apoptosis were all attenuated in rats fed tungsten or allopurinol diets. Cell transfer experiments provided additional evidence for the potential contribution of MNP XOR in the inflammatory process.

Methods. Most reagents, sodium tungstate, buffers, substrates, and inhibitors were purchased from Sigma Chemical Company (St Louis Mo., USA). Recombinant rat interleukin-1-α (IL-1 ; 500-RL-005) and interferon-y (IFN-γ; 285-IF-100) were purchased from R & D Systems (Minneapolis, Minn.). TUNEL staining kits were obtained from Trevigen, Inc. (Gaithersburg, Md.). Nitrotyrosine and immunoaffinity purified anti-nitrotyrosine antibody (IgG) were purchased from UpState Biotechnology (Lake Placid, N.Y.). Alexa Flour-488 and Alexa Flour-594 fluorescent antibodies were purchased from Molecular Probes (Eugene, Oreg.). Normal goat serum was obtained from ICN Biomedicals (Aurora, Ohio).

Intratracheal cytokine insufflation. Healthy male Sprague-Dawley rats (300-400 g body weight, Sasco, Omhaa, Nebr.) were fed control, tungsten enriched, or allopurinol supplemented diets as previously described (46,47). IL-1 and/or IFN-γ were delivered intratracheally into anesthetized rats as described previously (7). Dose-response optimization of IL-1 revealed no further inflammatory response to IL-1 at doses beyond 100 ng, and since the difference between 100 ng and 50 ng was small, 50 ng per rat was selected for use in the present experiments. Similar experiments and results were obtained with IFN-γ, which was subsequently tested in high and low dose in combination with IL-1. Routinely, 50 ng of recombinant rat IL-1α and/or 50 ng of IFN-γ in 0.5 mls of normal saline were pumped into the airway. Sham treated control rats were insufflated with normal saline alone. Differential and total inflammatory cell counts were determined on BALF obtained 24 hours following cytokine insufflation (6,7). TUNEL, nitrotyrosine staining, histology, and immunofluorescence were performed on lungs harvested 24 hours following cytokine insufflation. Lungs were perfused until blood free, removed surgically, and divided. One fraction was immediately fixed in paraformaldehyde for histology, TUNEL, or nitrotyrosine staining. One fraction was frozen immediately in liquid N₂ for subsequent biochemical analyses. Livers were obtained from each rat, perfused blood-free, and immediately frozen in liquid N₂. BALF cells were collected from separate rats by pumping 5.0 mls of normal saline into the trachea. Lavage fluid was pumped in and out of the lung three times before being collected. The use of rats in this study was approved by the University of Colorado Institutional Review Board under the protocol number 4980199(04)1E.

Xanthine oxidoreductase assay. Total XOR activity in lung and liver tissue was determined using whole tissue protein extracts. Briefly, tissues that had been perfused blood free were placed into liquid N₂ and stored at −80° C. prior to generating enzyme extracts. Enzyme extracts were prepared by placing frozen specimens in extract buffer (100 mM K-Phosphate, pH 7.8, 1 mM EDTA, 1 mM PMSF). Tissues were thawed in extract buffer on ice and minced. Minced tissues were broken in a rotating dounce using exactly three strokes of the pestle. Tissue homogenates were then centrifuged at 15,000×g at 4° C. for 30 min. Clarified extracts were desalted on 2 cm×20 cm Sephadex G25 columns to remove low molecular weight substances that potentially interfere with enzyme activity (48), and 3 ml of the flow through front were collected. Allopurinol inhibitable XOR activity was determined spectroscopically by measuring uric acid formation from xanthine at 290 nm. The stability of uric acid added to the flow through fraction was determined in the presence and absence of the uricase inhibitor, oxonic acid (OA). Because these analyses revealed high levels of uricase in rat lung and liver extracts, XOR assays were performed in the presence of 0.8 mM OA. Evaluation of D-form XOR and O-form XOR was determined by measuring enzymatic activity in the presence and absence of NAD+(49).

Tissue fixation, immunostaining, and microscopy. Lung tissue was fixed in 10% neutral-buffered formalin overnight and embedded in paraffin. Four micron sections were prepared and hydrated by exposure to xylene for 2—five minute periods followed by sequential 1 minute exposures to 100%, 90%, 70% and 30% ethanol and finally to phosphate buffered saline (PBS) for 5 min. Hydrated sections were either stained with H&E or prepared for antibody staining using the antigen retrieval procedure (Vector Labs, Burlingame, Calif.) according to the manufacturer's instructions. Sections were blocked and permeablized by incubation in PBS containing 0.2% glycine for 30 min followed by PBS containing, 5% goat serum and 0.1 mg/ml saponin for 1 hour and then incubated with anti-mouse XOR antibodies (1/100) at room temperature for 1 hour. Immunoreactivity was detected using a Cy 3 conjugated donkey anti-rabbit IgG (Jackson ImmunoResearch, West Grove, Pa.) secondary antibody (1/150) and visualized with a Nikon Diaphot inverted fluorescent microscope. Anti-XOR antibodies used for immuno-cytochemistry were generated against affinity purified XOR (49) and purified on Protein-A sepharose. Specificity of the XOR antibody was demonstrated by Western immunoblot analysis of crude tissue extracts (49).

Flow cytometric analyses. Cells were recovered from the BALF by centriflgation, washed in PBS, distributed at 2.5×105 cells in PBS/5.0% FCS into 96 well plates and then brought up to 100 uL with saline. All cells were pre-incubated for 5 min on ice with mouse anti-rat CD32 monoclonal clone D34-485 (BD Pharmingen, San Jose, Calif.) to minimize non-specific binding. Phycoerythrin (PE) labelled anti-rat CD11 b clone MRC OX-42 (Biosource International, Camarillo, Calif.) and biotin-conjugated anti-rat MNP monoclonal clone 1C7 (BD-Pharningen) were directly added to the wells 10 min in the dark on ice. The cells were washed in PBS/5.0% FCS followed by sedimentation for 5 min at 1200 rpm in a bench top Sorvall centrifuge and then incubated with either streptavidin-PE or streptavidin-FITC (both from BD-Pharmingen) in the appropriate wells. Subsequently, the cells were washed, fixed with 2% paraformaldehyde in PBS and then washed again. Reacted and washed cells were then resuspended in PBS and analyzed on a FACSCalibur analyzer (Beckton-Dickinson, San Jose, Calif.). The gates were set by a blank and the appropriate controls were used to indicate non-specific binding. A total of 10,000 cells were acquired for each sample and analyzed with Cell Quest (Beckton-Dickinson) version 3.1 software.

Lung nitrotyrosine staining. Paraffin embedded lung tissue sections were deparaffinized in ethanol and rehydrated in H₂O and PBS. Slides were blocked with a solution of 7.5% normal goat serum, 2.5% P-casein, 0.1% triton X-100 for 3 hrs at room temperature. The primary anti-nitrotyrosine antibody was applied at a dilution of 1:1 000 for 15 min at room temperature in the blocking solution. Slides were then washed in PBS and the Alexa Flour goat anti-rabbit IgG was applied for 15 min at a dilution of 1:1 00 in the blocking solution at room temperature. Subsequently, the slides were washed extensively in PBS and stained with Hoescht dye for 5 min in PBS, after which they were washed in PBS, covered in Anti-Fade, and sealed under glass coverslips. Positive immunofluorescent controls were exposed to 24 to 77 mM peroxynitrite for 20 min at room temperature, washed with PBS, and then processed as above. Pre-binding negative controls were performed by mixing the anti-nitrotyrosine antibody with 10 mM nitrotyrosine in PBS for one hour at room temperature prior to its addition to blocked slides. All subsequent steps were performed as above. Pre-binding blocked the reactivity of anti-nitrotyrosine antibody to nitrotyrosine but had no effect on heme-dependent autofluorescence of red blood cells. Slides were visualized under red (nitrotyrosine), green (tissue architecture), and blue (nuclei) fluorescence using a Nikon Diaphot inverted confocal fluorescence microscope at 100× magnification. Digitally derived blue and green photographs were combined with the MetaMorph software and printed from 24 bit digital files. A black background was set consistently for each figure using a section of the open air-space in which no cells were present in the field.

TUNEL staining and morphometric analysis. The TUNEL assay was used to detect apoptotic cells in fixed lung sections. After fixation with 4% buffered formalin, lung tissue sections were embedded in paraffin, sectioned at 5 uM, and mounted on glass slides. Slides were de-paraffinized, rehydrated for 10 min in PBS, and treated with 0.002% proteinase K (Sigma) in distilled water for 5 to 15 min at room temperature. Terminal deoxynucleotidyl transferase (TdT) was used for labeling of DNA nicks using the TACS kit (Trevigen, Inc., Gaithersburg, Md.). Counterstaining with DAPI was done to stain nuclear DNA and with Rhodamine coupled wheat germ agglutinin (WGA, Molecular Probes, Eugene, Oreg.) to stain the structural architecture. TUNEL positive cell staining appeared as green fluorescence in the nuclei of clearly defined alveolar cells. Background nuclear staining appeared as blue while the tissue architecture was red.

Activated caspase-3 detection. Detection of the cleaved, mature, activated caspase-3 was performed on paraffin-embedded sections using the Cell Signaling Technology, Inc (Beverly, Mass.) protocol. First, paraffin-embedded slides were deparaffinized and rehydrated. Incubation with 1% H₂O₂ for 10 min blocked endogenous peroxidase activity. For antigen unmasking, the tissue sections were microwaved in 10 mM sodium citrate buffer (pH 6.0) for 10 min. Next, the sections were blocked in 5% goat serum for 1 hr at room temperature. Slides were then incubated at 4° C. overnight with caspase-3 antibody specific for the cleaved, mature, form of caspase-3 diluted 1:200 in phosphate buffered saline (Asp-175; Cell Signaling Technology, Inc). This was followed by a 30 min incubation with a 1:200 dilution of goat anti-rabbit antibody (PK-6101; Vector Laboratories, Burlingame, Calif.). A subsequent 30 min incubation with VectaStain Elite ABC reagent (PK-6101; Vector Laboratories) ensued. This reaction was followed by the addition of the DAB substrate kit for peroxidase (SK-4100; Vector Labs). The slides were finally counterstained with hematoxylin (H-3401; Vector Labs), dehydrated and mounted. Caspase-3 positive cells were quantitated by counting the percent positive alveolar cells per field under 100× magnification.

SDS-PAGE and immunoblot analysis. Protein was electrophoresed on SDS-PAGE and transferred to nitrocellulose membranes (Osmonics, Inc, Minnetonka, Minn.). Membranes were sliced for staining with commassie brilliant blue or processed for immunoblot analysis. For reaction with antisera, membrane strips were blocked with 0.05% dried milk overnight prior to reaction with preimmune sera or anti-XOR antisera. Antigen-antibody complexes were detected by reaction with an ECL Western blotting detection kit according to manufacturer's instruction (Amersham Life Sciences, Piscataway, N.J.).

Statistical analyses. Data are expressed as the mean and standard error of the mean and were assessed for significance using the Student's t-test. A p value of <0.05 was considered significant.

Results

XOR was Induced in the Lungs of Rats Following IL-1 and IFN-γ Insufflation

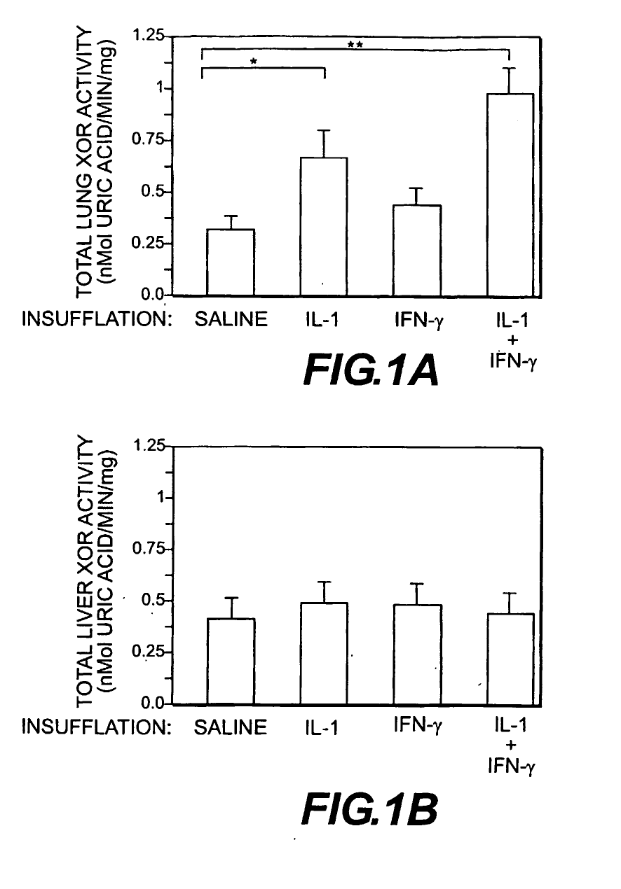

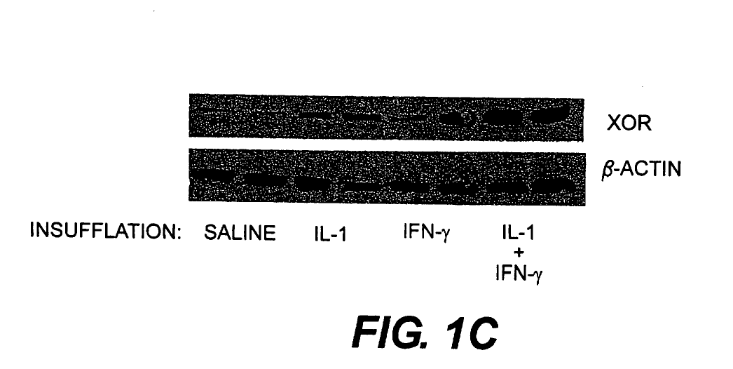

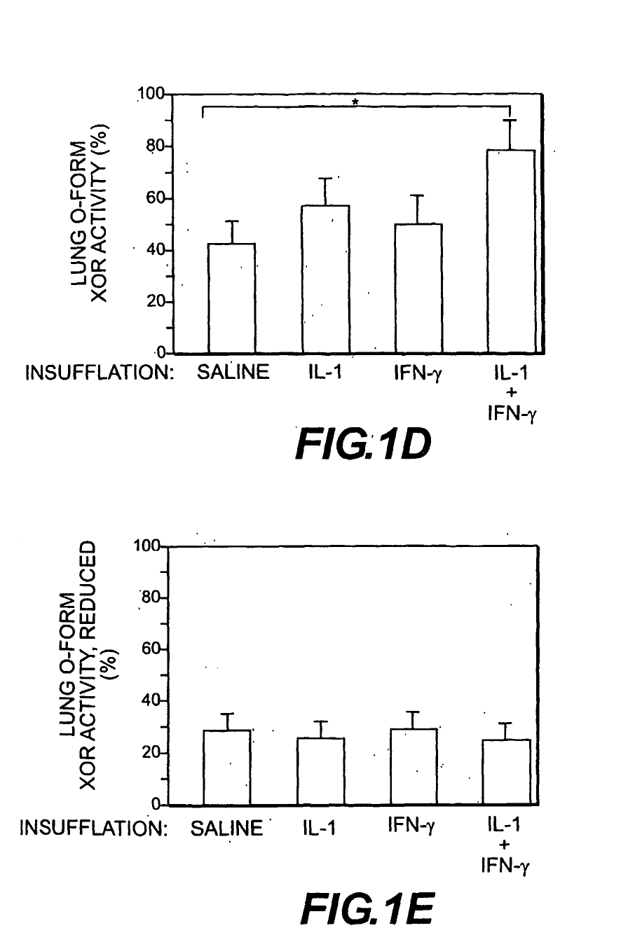

XOR was quantitated in desalted whole-lung protein extracts obtained from lungs and livers of rats subjected to intratracheal insufflation with either IL-1 and IFN-γ, IL-1, IFN-γ, or saline (the cytokine vehicle). The combined insufflation of IL-1 and IFN-γ produced a three-fold induction of total XOR activity 24 hours following cytokine insufflation compared to saline alone (FIG. 1a). By comparison, there was an approximate doubling of XOR after IL-1 insufflation and a negligible response following IFN-γ insufflation alone. In contrast, liver XOR did not increase following cytokine insufflation (FIG. 1b). Western immunoblot analysis of lung protein extracts showed that lung XOR protein also increased several fold following insufflation with IL-1 and IFN-γ (FIG. 1c). XOR recovered from cytokine insufflated lung revealed no evidence of elevated proteolysis.

XOR was Recovered in Predominantly O-Form from the Lungs of Rats Insufflated with IL-1 and IFN-γ

We quantitated the levels of O-form XOR in untreated or cytokine insufflated rat lungs. Untreated, normal rat lungs exhibited 23% O-form XOR, an amount that is typical of most rat tissues, while O-form XOR increased to 78% of the total XOR activity in lungs insufflated with IL-1 and IFN-γ. In contrast, lung O-form content increased to 42% following saline insufflation, 57% following IL-1 insufflation and 52% following IFN-γ insufflation (FIG. 1d). O-form XOR recovered from cytokine insufflated lungs was in a reversible state. Soluble lung protein extracts from insufflated rats were exposed to 5 nM DTT at 37° C. for one hour and were re-chromatographed on sephadex G25 after which D-form and O-form content was re-measured. Regardless of insufflation, O-form XOR in all lung samples was reduced to approximately 23% of the total XOR by DTT (FIG. 1e). Furthermore, native O-form XOR from untreated rat lungs or livers was not further reduced by DTT treatment and remained at approximately 23% of the total XOR following DTT reduction.

Lung Inflammation Increased Following IL-1 and IFN-γ Insufflation

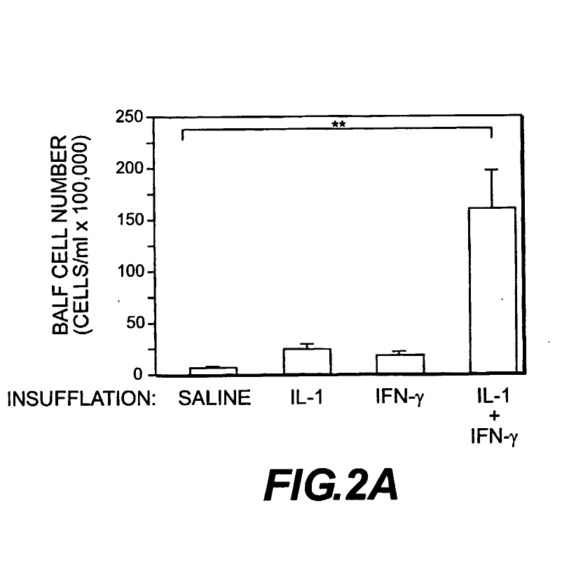

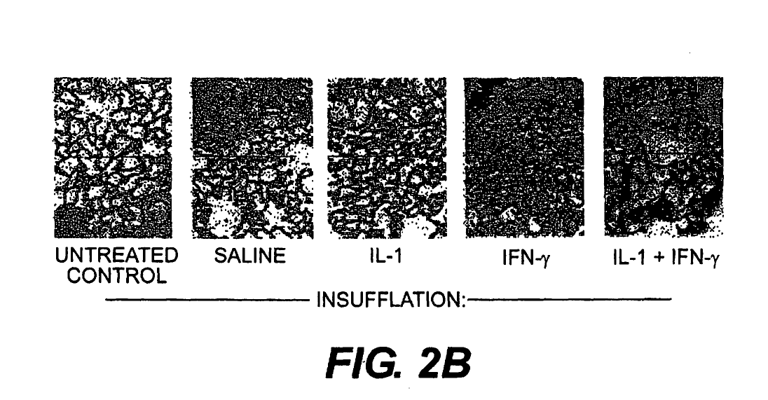

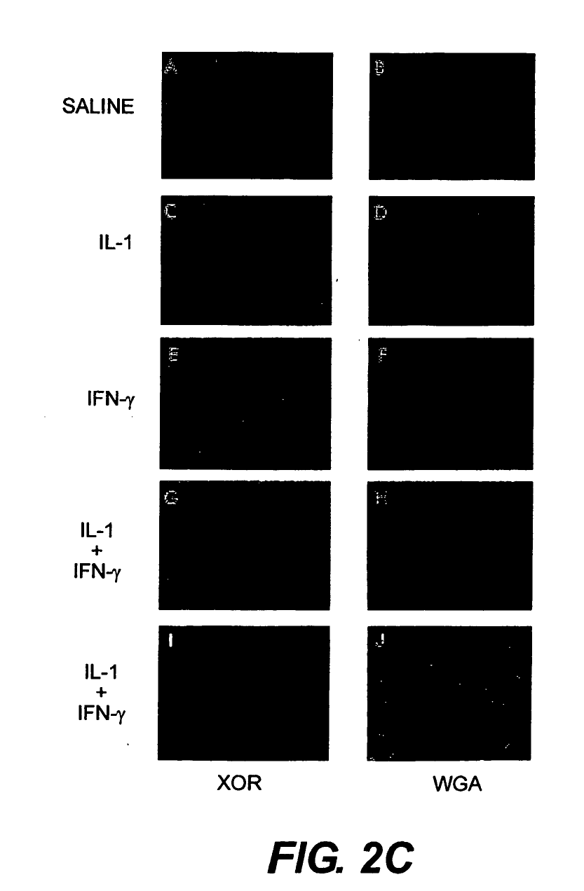

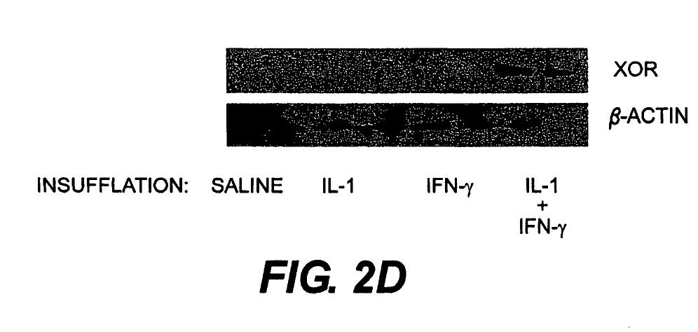

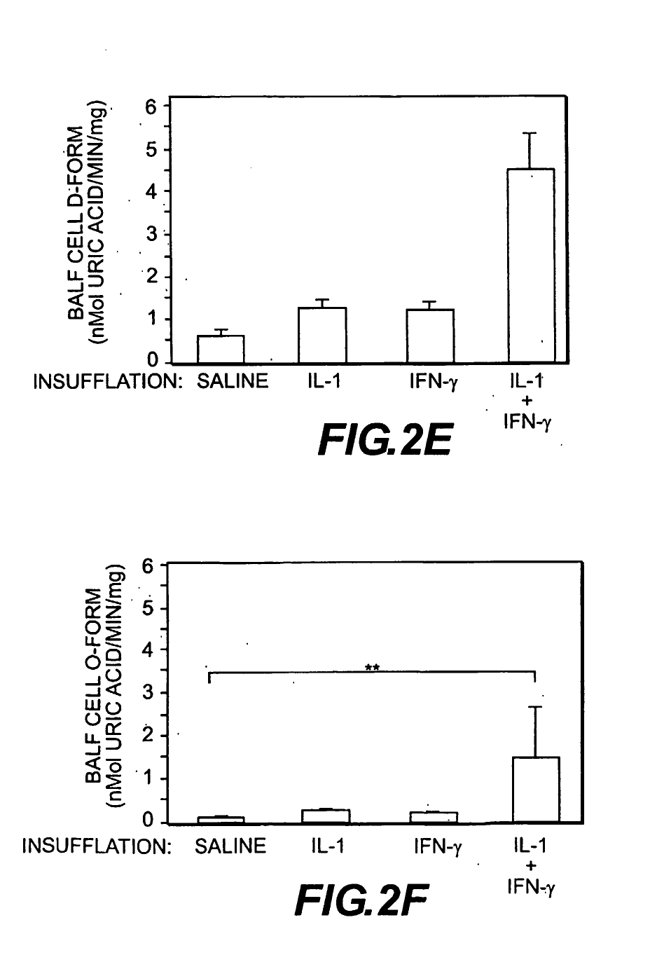

The total number of inflammatory cells recovered from the BALF 24 hours following IL-1 and IFN-γ insufflation was increased compared to saline, IL-1, or IFN-γ insufflated lungs (FIG. 2a). Differential analysis revealed approximately 60% neutrophils and 40% macrophages in BALF from IL-1 and INF-γ insufflated lungs compared to approximately 80% neutrophils and 20% macrophages from IL-1 or INF-γ insufflated lungs and essentially 100% macrophages from non-insufflated lungs of control rats. Lymphocytes were a small, but comparable, percentage of the cells recovered in all instances. Histological examination of cytokine insufflated lungs confirmed the increase in inflammatory cells recovered in BALF from the lungs of IL-1 and IFN-γ insufflated rats and revealed increased numbers of inflammatory cells in the airway and in the perivascular regions of the lung (FIG. 2b). The cellularity observed histologically with IFN-γ alone appears greater than the cells recovered in the BALF, suggesting a possible interstitial localization of MNP in the IFN-γ insufflated lungs.

XOR was Induced in Lung Inflammatory Cells Following IL-1 and IFN-γ Insufflation.

The predominance of O-form XOR in IL-1 and IFN-γ insufflated rat lungs was surprising since XOR exists in native lung tissue in predominantly D-form. Accordingly, we sought to identify lung cells that expressed XOR following cytokine insufflation. Immunofluorescent staining of rat lung tissue preparations demonstrated that XOR was induced primarily in inflammatory cells following IL-1 and IFN-γ insufflation (FIG. 2c). By comparison, XOR immunoreactivity was virtually undetectable in lungs following saline, IL-1, or IFN-γ insufflation. Western immunoblot analysis indicated that XOR was induced dramatically in the BALF cells by the combined action of IL-1 and IFN-γ compared to the response produced by saline, IL-1, or IFN-γ insufflation (FIG. 2d). D-form (FIG. 2e) and O-form (FIG. 2f) XOR activity were also elevated in BALF cells recovered from rats insufflated with IL-1 and IFN-γ compared to the response to saline, IL-1, or IFN-γ insufflation. Furthermore, predominantly O-form XOR was again recovered in the inflammatory cells recovered from IL-1 and IFN-γ insufflated rat lungs.

XOR was Induced in the Infiltrating and Differentiating MNP Following IL-1 and IFN-γ Insufflation.

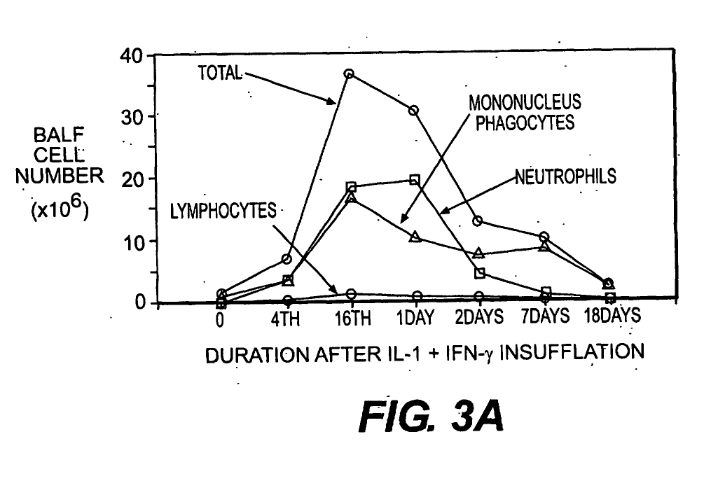

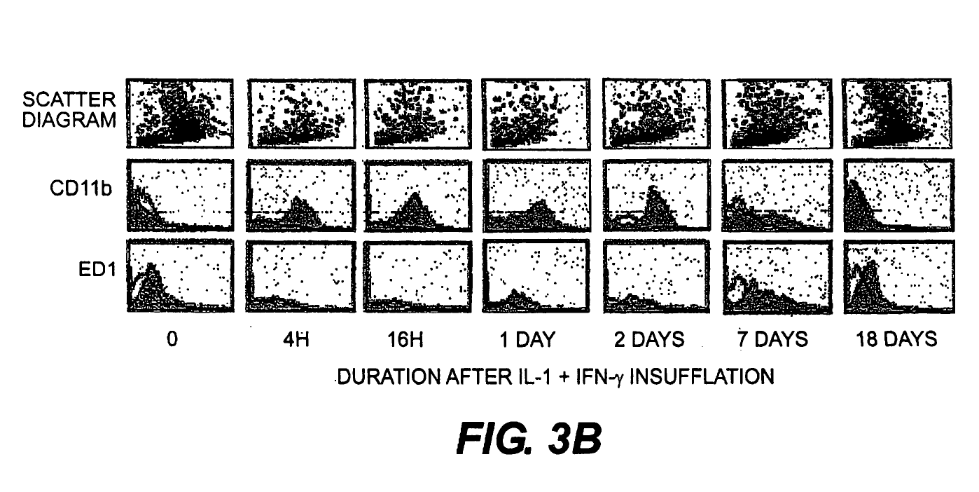

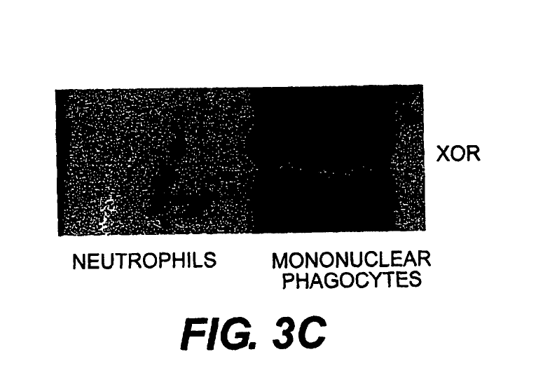

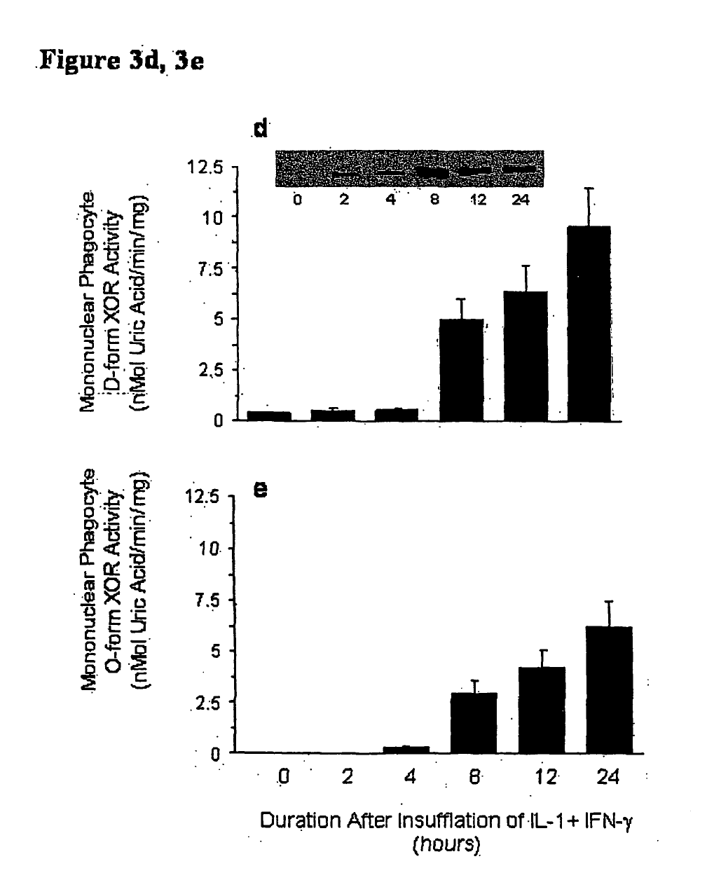

Differential analysis of cells obtained in the BALF following IL-1 and IFN-γ insufflation revealed nearly equivalent numbers of MNP and neutrophils by 24 hours that declined slowly over the next 18 days (FIG. 3a). BALF cells from IL-1 and IFN-γ insufflated rats were stained for the alveolar macrophage marker, ED1, or with the neutrophil/monocyte marker, CD11b, and were then subjected to analysis by FACS (FIG. 3b). The CD11b antigen increased dramatically 4 hours following cytokine exposure, reflecting the appearance of newly infiltrating CD11b positive monocytes and neutrophils. CD11b staining then gradually declined over the next 18 days to a pattern identical to that found in the 0 time resident macrophages. ED1 was well expressed on the 0 time resident macrophages, was absent on the newly infiltrating monocytes and neutrophils at four hours, and was gradually restored to high level expression throughout the 18 day time course. These data reflect the rapid migration of neutrophils and monocytes into the lung following IL-1 and IFN-γ insufflation, followed by the gradual maturation of the newly infiltrating monocytes into mature, ED1 expressing, macrophages and by the relatively rapid decrease of the neutrophil population. Scatter diagrams revealed the broad forward and side scatter produced by resident macrophages at time 0, and subsequently revealed the low scattering, compact nature of infiltrating monocytes and neutrophils, a pattern that was restored to the original pattern typical of the mature macrophages over the course of 18 days.

Neutrophils and MNP were recovered from the BALF 24 hours following IL-1 and IFN-γ insufflation and purified on Percoll gradients. Western immunoblot analysis of protein extracted from these cells showed that XOR was induced in MNP, but not neutrophils (FIG. 3c). BALF cells were also recovered over a 24 hour time course from rats following insufflation of IL-1 and IFN-γ, collected by centrifugation and washed. MNP were then allowed to adhere to plastic dishes by cultivation at 37° C. for one hour in rich medium. Non-adherent cells were then washed off of the plates and the adherent MNP were assayed for D-form and O-form XOR activity and for XOR immunoreactive protein. These data demonstrated that D-form XOR (FIG. 3d) and O-form XOR (FIG. 3e) activity were induced in the adherent MNP, and that most of this XOR was recovered in O-form. Although XOR was induced over a 24 hour period in the infiltrating MNP recovered from the lungs of IL-1 and IFN-γ insufflated rats, neither XOR activity or antigen was detected in circulating rat monocytes (not shown). These observations indicate that XOR induction is associated with monocyte infiltration and/or differentiation following cytokine insufflation.

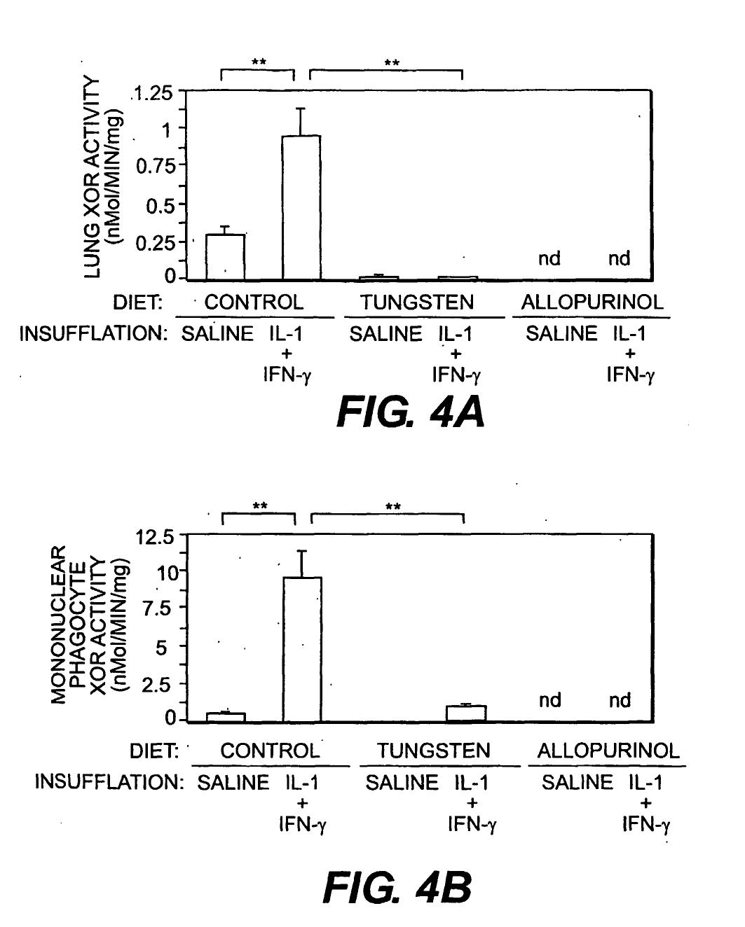

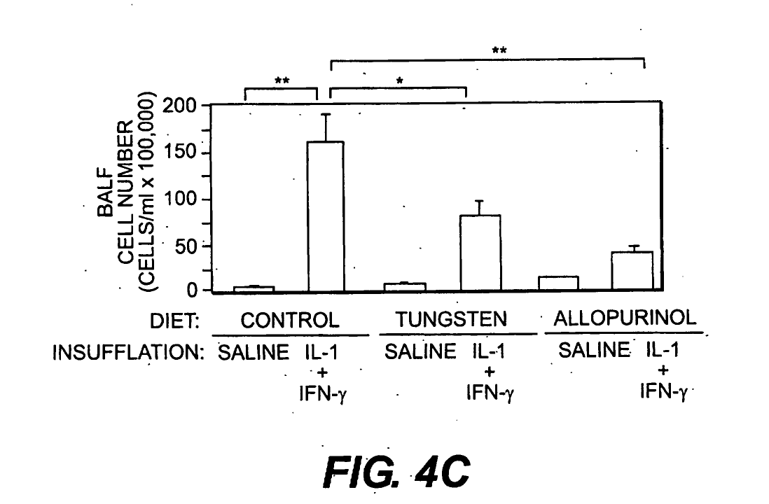

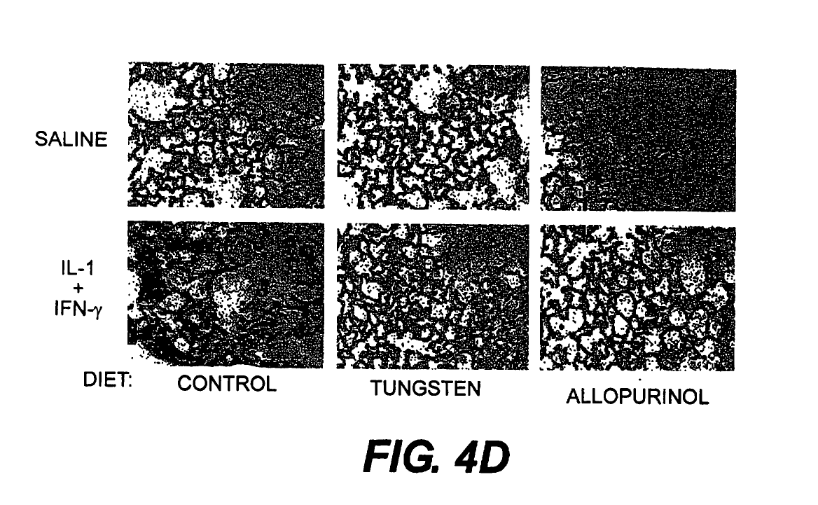

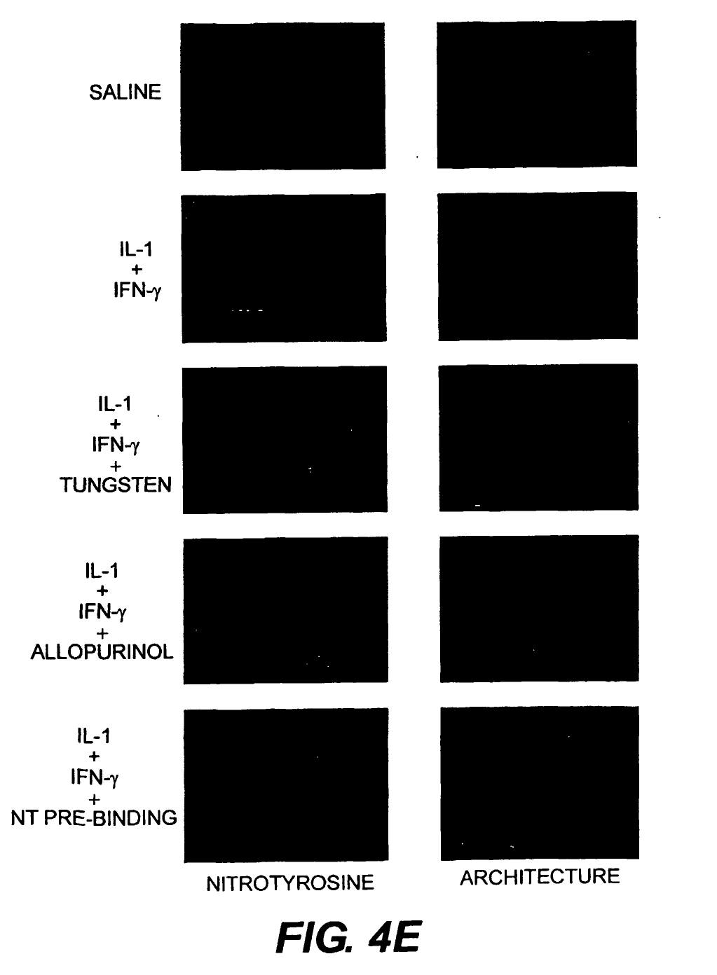

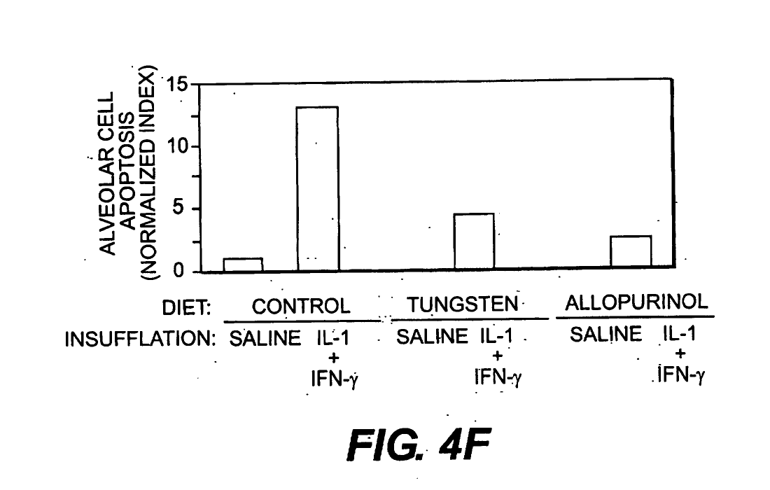

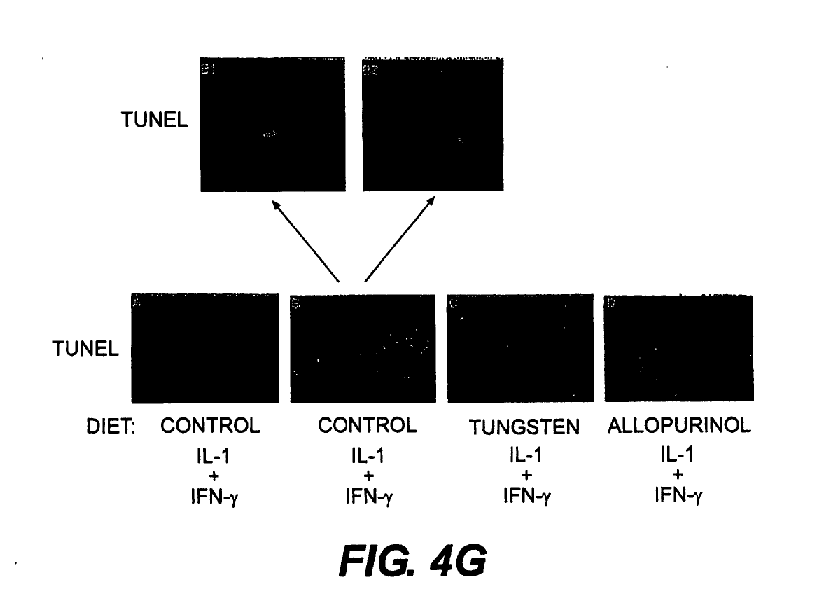

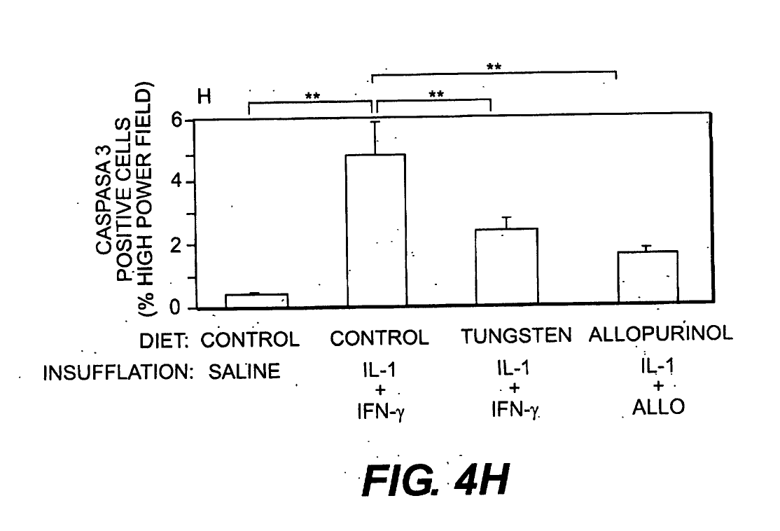

Tungsten or allopurinol feeding decreased lung and MNP XOR activity, lung inflammation, MNP nitrotyrosine staining, and alveolar cell apoptosis in rats insufflated with IL-1 and IFN-γ. The induction of XOR in MNP recovered from lungs following IL-1 and IFN-γ insufflation and its conversion into largely O-form XOR suggested that XOR may participate in the inflammatory process as a source of ROS. We used two systemic inhibitors to assess the involvement of XOR in cytokine induced inflammation. Rats were fed diets deficient in molybdenum and supplemented with tungsten or diets supplemented with allopurinol. Subsequently, rats were insufflated with IL-1 and IFN-γ or the saline vehicle and 24 hours later prepared for analysis. Tungsten feeding decreased XOR activity in the lungs (FIG. 4a) and in the BALF cells (FIG. 4b) from cytokine insufflated rats. Furthermore, tungsten or allopurinol feeding also reduced the accumulation of inflammatory cells (FIG. 4c) and attenuated histologic evidence of airway and perivascular inflammation (FIG. 4d) in the lungs of IL-1 and IFN-γ insufflated rats. Lung tissue specimens from rats insufflated with IL-1 and IFN-γ exhibited increased nitrotyrosine staining of the alveolar MNPs (88% positive staining) compared to saline insufflated control lungs (14% positive staining), and MNP nitrotyrosine staining was decreased to background levels (14% positive staining and autofluorescent cells) in IL-1 and IFN-γ insufflated rats previously fed allopurinol or tungsten diets (FIG. 4e). Finally, since a common feature of ALI and a consequence of inflammation can be the induction of alveolar cell apoptosis, we examined cytokine insufflated rat lungs for apoptosis. By quantitative morphometric analysis, lungs from rats insufflated 24 h before with IL-1 and IFN-γ had a 13 fold increase in TUNEL positive alveolar nuclei staining compared to saline insufflated rat lungs and prior feeding with tungsten or allopurinol diets attenuated the development of alveolar apoptosis (FIG. 4 f,g). We quantitated activated caspase-3 in a similar fashion to corroborate the results from TUNEL assay. Lungs from rats insufflated 24 hours before with IL-1 and IFN-γ had a 12 fold increase in activated caspase-3 compared to saline insufflated control lungs. Prior feeding with tungsten or allopurinol diets blocked the activation of caspase-3 in cytokine insufflated rat lungs (FIG. 4h).

MNP XOR Contributed to the Inflammatory Response in vivo.

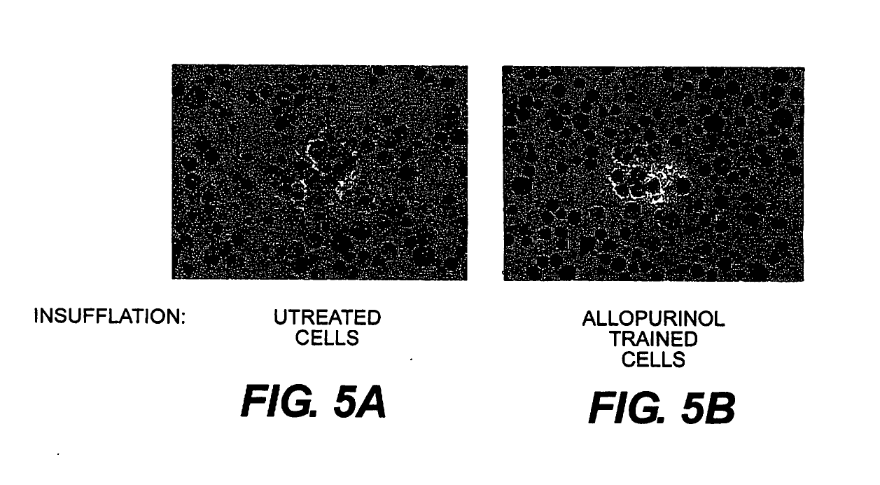

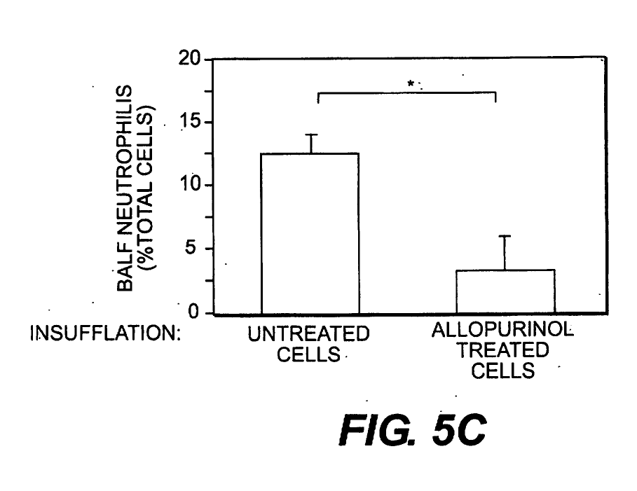

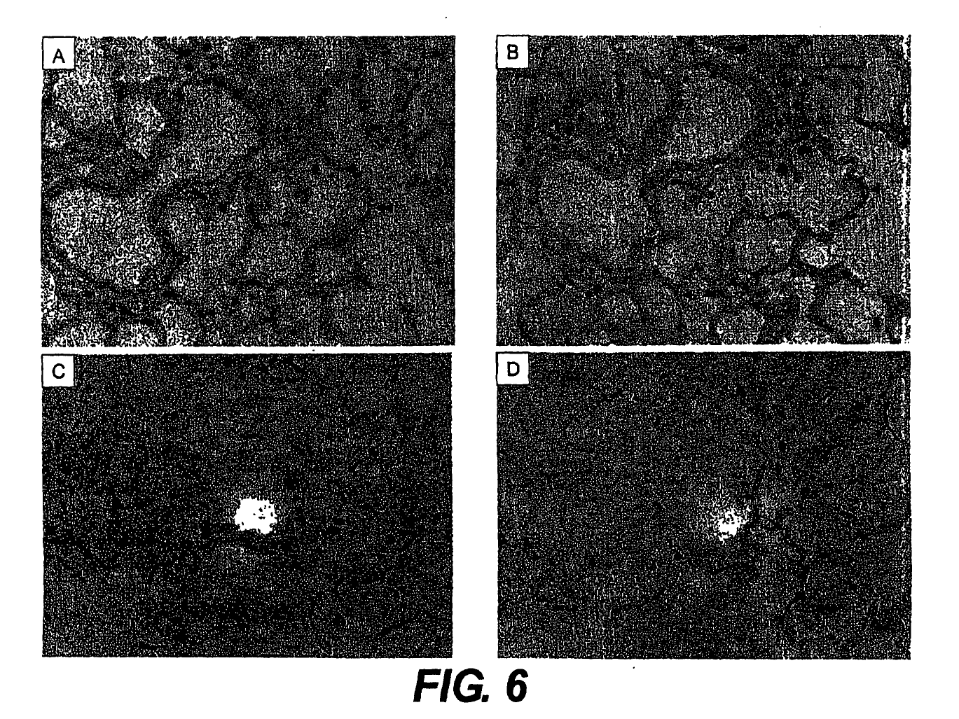

To determine whether XOR induction in MNP could contribute to lung inflammation, we performed a cell transfer experiment. We induced a pulmonary inflammatory response in rats with IL-1 and IFN-γ insufflation, obtained these cells by bronchoalveolar lavage 8 hours later, and then treated these cells with either allopurinol or the vehicle for allopurinol in vitro. After a 15 min exposure to allopurinol or the vehicle, the cells were washed, resuspended in PBS, and then insufflated into control rats. Subsequently, cells were harvested from the BALF of these control rats, stained, and quantitated. BALF recovered from the lungs of rats insufflated 24 hours before with untreated cells had increased numbers of neutrophils compared to BALF recovered following insufflation with allopurinol pretreated cells (FIG. 5a,b). Insufflation of allopurinol treated BALF cells attenuated the subsequent recovery of neutrophils in the BALF (FIG. 5c).

Discussion

We observed the rapid induction of XOR in the infiltrating, differentiating MNP that were recruited into the lungs of rats insufflated with IL-1 and IFN-γ. Lungs of rats insufflated with IL-1 and IFN-γ developed a vigorous inflammatory response characterized by rapid neutrophil and MNP infiltration, oxidative stress, and alveolar cell apoptosis. MNP XOR was recovered in predominantly its reversible O-form, and MNP from the lungs of IL-1 and IFN-γ insufflated rats had increased nitrotyrosine staining compared to MNP in control lungs. Pretreatment of rats with two different XOR inhibitors decreased MNP XOR induction, MNP nitrotyrosine staining, lung inflammation, and alveolar cell apoptosis. Transfer of allopurinol inhibited MNP into normal rat lungs in vivo decreased the recruitment of neutrophils into the lung. While vascular endothelial cell XOR may contribute to inflammation (35), our observations point to a dynamic, previously unrecognized, process by which XOR induction in the MNP contributes to lung inflammation, oxidative stress, and alveolar cell apoptosis.

BALF from untreated rats contained primarily alveolar macrophages and few, if any, neutrophils or monocytes. These resident macrophages had high levels of the macrophage marker, ED1, but expressed low levels of XOR immunoreactive protein and activity. However, this pattern changed rapidly after cytokine insufflation. Beginning four hours after cytokine insufflation, CD11b expressing, ED1-deficient cells exhibiting the compact, low scattering pattern characteristic of monocytes and neutrophils dominated the population of cells recoverable by lung lavage. Over the initial 24 hours, XOR expression increased in the CD11b-positive ED1-negative MNP. This pattern gradually reverted to the zero time control pattern over the next 18 days when a majority of the recoverable cells were again ED1-positive macrophages. Simultaneous histologic and biochemical analyses of these BALF cells revealed that XOR activity was increased in MNP, but not neutrophils. Because XOR activity was not detected in circulating monocytes, the present observations suggest that increased XOR expression occurred in the infiltrating and differentiating MNP.

Predominantly O-form XOR was recovered in MNP throughout the 24 hours following IL-1 and IFN-γ insufflation. As early as 8 hours post-insufflation, O-form content comprised 70-80% of the total XOR activity. Importantly, O-form XOR could be reversed to D-form XOR by DTT reduction leaving a constant O-form content of about 23% regardless of cytokine exposure, and this is exactly the level of O-form XOR found in the lungs of untreated rats or in livers. Thus, the increased amount of O-form XOR recovered from the lung was dependent upon prior cytokine insufflation. Since we observed no increase in XOR proteolysis following cytokine treatment, the combined action of IL-1 and IFN-γ elevated both total XOR activity and most likely promoted the reversible conversion of D-form XOR to the O-form in vivo. Although inflammatory cytokines increased XOR in cultured epithelial cells, they did not cause conversion of D-form to O-form XOR in vitro (42). While these contrasting observations undoubtedly reflect the different response of immortalized cells in culture and in the rat lung in vivo, the increase in XOR along with the increase in O-form content could enhance the ROS generating capacity of MNP in the alveoli.

The development of increased alveolar MNP nitrotyrosine staining following IL-1 and IFN-γ insufflation suggests that oxidative stress was increased in MNP, and nitrotyrosine modification can be supported by O-form XOR which can serve as source of O₂⁻ (34-36). Inhibition of MNP nitrotyrosine staining in rats fed tungsten or allopurinol indicates that XOR served as a source Of O₂⁻ in the MNP following cytokine insufflation. Although other sources of O₂⁻ exist in MNP, they are unlikely to be inhibited by both tungsten and allopurinol. We observed that 88% of the alveolar MNP stained positive for nitrotyrosine following cytokine insufflation, with a background of 14% positive MNP. The increase of 74% due to cytokine induced inflammation is nearly identical to the level of nitrotyrosine staining seen at the onset of ARDS in humans (50), and suggests that oxidative stress in the MNP may be a general, early feature of acute lung inflammation.

In addition to inducing XOR expression in the MNP and generating an inflammatory response, insufflation of IL-1 and IFN-γ also increased alveolar cell apoptosis that was quantitated by TUNEL stain and caspase-3 activation. Alveolar cell apoptosis has been frequently observed in clinical ALI and in experimental lung inflammation and appears to depend on activation of the Fas/FasLigand proteins (51-52) which are highly expressed on both inflammatory cells and lung epithelial cells (53). While the exact mechanism responsible for alveolar cell apoptosis following cytokine insufflation is unclear, the inhibition of alveolar cell TUNEL stain and caspase-3 activation by tungsten or allopurinol treatment implicates the involvement of XOR.

We established the contribution of XOR to cytokine induced lung inflammation using tungsten and allopurinol inhibition. Both of these inhibitors decreased lung inflammation, MNP nitrotyrosine staining, and alveolar cell apoptosis. Allopurinol is a highly specific inhibitor in vivo that inhibits XOR noncompetitively following its conversion into oxypurinol (alloxanthine) which then inhibits the molybdenum center by tight, but reversible, binding (54). In our experiments, rats were fed allopurinol at a dose of 50 mg/Kg for seven days, a regimen well known to inhibit XOR in vivo (26,27). While high concentrations of allopurinol may theoretically have ROS scavenging capability in vivo (55), this results primarily from quenching hydroxyl radical and not O₂⁻ (56). Tungsten feeding is another relatively selective method for inhibiting XOR in vivo (26-29). Tungsten acts by displacing molybdenum from the molybdopterin cofactor necessary for the activity of XOR and other MoCo enzymes (57). The similar effect achieved by treatment with either of these distinct inhibitors indicates that XOR activity contributed to the cytokine induced inflammatory response, MNP nitrotyrosine staining, and alveolar cell apoptosis seen in rats insufflated with IL-1 and IFN-γ.

Because the use of systemic inhibitors like tungsten or allopurinol would not be limited to inhibiting XOR in the MNP, we performed a cell transfer experiment in which XOR was inhibited in MNP in vitro. Insufflation of allopurinol treated, XOR inhibited, MNP demonstrated that MNP XOR could contribute to cytokine induced lung inflammation. BALF cells recovered from rats insufflated with allopurinol treated cells had reduced numbers of neutrophils compared to the numbers of neutrophils in the BALF following transfer of cells treated with the vehicle alone. This experiment indicated that XOR specifically located in MNP participated in the pulmonary inflammatory process. These experiments do not exclude the possible contribution of endothelial or epithelial XOR to the inflammatory process, nor do they address the potential role of interstitial or parenchymal MNP. In fact, our data suggest that substantial levels of interstitial MNP arose following IFN-γ insufflation that were not recovered in the BALF. Nonetheless, our data do point to the unanticipated participation of alveolar cell MNP XOR in cytokine induced lung inflammation and underscore a possible functional interaction between neutrophils and MNP that is dependent on XOR.

Our study demonstrates that XOR can be induced rapidly in infiltrating and differentiating MNP following cytokine insufflation in rats and that this phenomenon is linked to the inflammatory process. We imagine that MNP XOR generates ROS that contribute to increased pulmonary oxidative stress and inflammatory cell recruitment. These observations may explain the oft-postulated “two hit” process leading to the exuberant inflammation characteristic of ALI. If XOR is increased in MNP for as long as 18 days after an initial cytokine or other insult, then the lung may be more susceptible to a second insult for a considerable time. Whether allopurinol would be beneficial in treating or preventing ALI or other inflammatory diseases is unknown. However, the protective effect of allopurinol in diabetes (58,59), renal ischemia-perfusion injury (60), and chronic heart failure (61-63) may be related to this mechanism inasmuch as MNP infiltration appears to be a component of each of these diseases.

### Example 2

Granulomatous lung inflammation (GLI). GLI is a significant health problem reported to affect 10 to 40 individuals per 100,000 each year in the USA alone, with a three fold bias in the black population. GLI is the primary pathogenic mechanism underlying sarcoidosis, although many organs can be affected by the granulomatous inflammation of sarcoidosis (161). GLI/sarcoidosis is a common inflammatory disorder of complex etiology that arises from the induction of a focal inflammatory response in the lung (and elsewhere) and has a significant fatality rate. While human disease is frequently resolved with prednisilone therapy, for unknown reasons this is frequently not successful. However, recent clinical trials in humans have demonstrated the protective effect of allopurinol, an XOR inhibitor, which has been shown to regress late stage cutaneous and pulmonary sarcoidosis. This observation links sarcoidosis to several other human inflammatory diseases in which allopurinol has also proved beneficial. Our studies will provide insight into the mechanisms by which XOR contributes to the pathogenesis of GLI/sarcoidosis and, hopefully, provide new approaches for treating and/or preventing this inflammatory disorder. Rats are being used because they provide a way of testing in vivo in a relevant physiologic way the findings found previously with in vitro analyses or established phenomenologically in humans. Because GLI patients can be very sick and the disease process in humans is very complex, only a limited amount of information can be obtained from studies in humans. In addition, the ability to assess any therapeutic approach is very difficult and expensive in human patients since many parameters of treatment have not been established (for example: optimal XOR blockers, timing, route of administration etc.). Rats have been utilized in prior studies of granulomatous inflammation using intravenous BCG or TDM (trehalose-6,6′-dimycolate) to induce lung granuloma and, consequently, our studies will be directly comparable to these prior analyses. All rats will be housed and cared for in the Webb-Waring Institute's own AAALAC accredited animal care facility.

The pathophysiologic course of GLI/sarcoidosis is typified by a panoply of infectious or antigen sources that incite a Th-1 lymphocytic response that in turn promotes chronic, focal lung inflammation characterized by T-cell/mononuclear phagocyte (MNP) infiltration, cytokine elevation, and granuloma formation (161-164). While both T-helper-1 and T-helper-2 T-cells (Th-1 and Th-2) appear to contribute to the development of granuloma (165,166), the Th-1 response appears to stimulate subsequent MNP influx and granuloma formation (165,166). In human disease, chronic Th-1 stimulation may play a central role in the persistent recruitment and differentiation of MNP into mature macrophages and multinucleate giant cells (MGC) (166,167) with the consequent growth of the granuloma and development of mononuclear cell alveolitis and parenchymal infiltration (166,168). MNP alveolitis and dense packing of MNP in the granuloma are now considered to be defining features of GLI/sarcoidosis (161-164). Importantly, cells obtained in the bronchoalveolar lavage fluid (BALF) of patients with active disease were dominated by MNP showing high levels of the monocyte markers CD11a and CD11b suggesting disruption of MNP differentiation or the presence of MNP in different stages of differentiation (166).

Oxidative stress may contribute to the pathophysiology of GLI/sarcoidosis and other inflammatory lung injuries. Evidence of ongoing oxidative stress has been commonly observed clinically in GLI and related chronic inflammatory lung diseases and involves diverse markers of oxidative stress (169-171). Oxidized protein in the BALF (169), enhanced nitric oxide and inducible nitric oxide synthase (170), and elevated antioxidant enzymes are all indicative of increased oxidative stress associated with sarcoidosis (171). Furthermore, MNP from the BALF of GLI/sarcoidosis patients produce excess ROS compared to controls, unlike circulating monocytes from the same patients (172-174), suggesting that the MN? must first enter the airspace to generate substantial ROS. PMA stimulated expression of ROS from MNP recovered in the BALF of sarcoidosis patients was further enhanced by exposure to interferon-γ (IFN-γ) (175), a defining cytokine of the Th-1 response. Spontaneous and induced release of superoxide anion from MNP cells of sarcoidosis patients has been linked to those cells expressing adhesion molecules indicative of immature MNP (176), suggesting either disruption of MNP differentiation or the continuous influx of new monocytes. While the consequences of increased ROS generation from the alveolar MNP of sarcoidosis patients can be very broad (177), the sources of ROS in the MNP are still not fully defined. In the related chronic, diffuse inflammation of COPD oxidative stress is a well recognized feature (178), and xanthine oxidoreductase has been identified as a critical mediator of ROS generation in COPD (179-181). Furthermore, both the skin and pulmonary manifestations of sarcoidosis have been consistently suppressed by treatment with the XOR inhibitor, allopurinol, and this now an accepted treatment for clinical sarcoidosis (182-186). Thus, it can be concluded that ongoing oxidative stress is an important feature of chronic lung inflammatory states and of GLI/sarcoidosis in particular, and XOR may be a significant, albeit poorly understood mediator of GLI/sarcoidosis.

Cytokines elevated in the lungs of GLI/sarcoidosis patients and released from inflammatory cells, epithelial cells, fibroblasts, and other cells of the lung could promote XOR dependent oxidative stress. Cytokines and other factors that are increased in the bronchoalveolar lavage fluid (BALF) of GLI patients include (in part) TNF-α (219,220), IL-1 (221-223), IFN-γ (162,163), MCP-1 (224,225), MIP-1α (226), MIP-1β (224,226), MIP-3β (227), and TGF-β (228). While these cytokines and chemokines appear to determine the clinical progression of GLI and its outcome, they may be equally critical for the induction and expression of XOR. It can well be imagined that cytokine activation of XOR is a necessary prerequisite for its involvement in an inflammatory process, and two key points about cytokines and XOR are worth considering.

Cytokines that are elevated in GLI can increase lung XOR expression and subsequent ROS production. In particular, IL-1 is increased in the lungs of GLI patients and may contribute to inflammation by inducing the expression of inflammatory chemokines (MCP-1) and adhesion molecules important for migration of inflammatory cells into the air space (ICAM-1) (221-223). Delivery into rats of IL-1 by intratracheal insufflation or as a stable transgene in an adenoviral backbone promotes both acute and chronic inflammatory cell accumulation and lung leak (223,229). Furthermore, IL-1 induces expression of inflammatory chemokines (MCP-1) and adhesion molecules (ICAM-1) in cultured lung epithelial cells in vitro (230). Importantly, XOR steady state RNA and enzyme levels are regulated in several cultured cells by IL-1 through a cyclohexamide and an actinomycin-D inhibitable mechanism (214-216). IL-1 alone induces XOR by approximately 4 fold in cultured endothelial and epithelial cells (215,216). As a signature cytokine of the Th-1 response, IFN-γ is also increased in the BALF of GLI patients (162,163) and induces inflammatory cell recruitment in vivo following intratracheal insufflation (231). IFN-γ also induces XOR steady state RNA, protein, and enzyme activity by mechanisms potentially synergistic with IL-1 in cultured cells in vitro and in vivo in mice (214-216,232,233). While cultured epithelial and endothelial cells express predominantly D-form XOR, the potential for XOR to be converted to its ROS generating O-form by cytokines in vivo is an important consideration that will be assessed.

Expression of XOR in the inflammatory MNP population may potentiate or amplify the inflammatory process of GLI/sarcoidosis in the lung, and this underlies the protective effect of allopurinol. Mononuclear phagocytes (MNP) and T-lymphocytes (Th-1 and Th-2 cells) play essential and distinct roles in GLI/sarcoidosis (161-164). CD4+ and CD8+ T-cells have been recognized as important mediators of the alveolar inflammatory process and granuloma formation that can interact with MNP cells, alveolar epithelial cells, or perhaps B-cells to alter chemokine expression and locally amplify the inflammatory process (236,237). The role played by CD4+ and CD8+ Th-1 lymphocytes in the pathophysiology of GLI is complex. We imagine that recognition of MNP presented antigen, or possibly epithelial cell presented antigen, by locally recruited Th-1 cells results in T-cell activation and expression of IFN-γ which in turn further promotes MNP activation, chemokine release, amplification of the inflammatory response, and growth of the granuloma. Many of these steps are still poorly understood.

MNPs play key roles in the chronic inflammatory process of GLI/sarcoidosis. The participation of MNPs in GLI is not well understood, however, MNP may serve important roles at several stages. First, resident alveolar macrophages (AM) may be the primary cell recognizing and presenting antigen to Th-1 cells. AM are also highly sensitive to IFN-γ activation and, once activated, are potent sources of pro-inflammatory cytokines such as TNF-α, MCP-1, IL-1, MIP-1, TGF-β or reactive oxygen species (172-176). Resident, mature AM comprise close to 100% of the cells found in the BALF of resting, untreated rats, and when detected, lymphocytes comprise well below 1% of the total inflammatory cell population. Thus the resting lung may be considered to harbor an essentially pure population of mature macrophages. The resting mature AM responds to many activating signals including products of antigen primed Th-1 cells like IFN-γ (238,239). Upon activation, AM secrete cytokines involved in the orchestration of the inflammatory response, activate the respiratory burst NADPH oxidase, and release ROS. For example, IL-1, and TNF-α are released in response to IFN-γ (238). IFN-γ signal transduction in the macrophage operates through a system of protein relays that involves activation of the JAK/STAT pathway and NF-κB activation (240), and XOR is now know to be regulated by the JAK/STAT system (217,234). Thus, a reasonable view of the resident AM would imagine these cells to be highly responsive to pathogen by processing and presenting antigen to Th-1 cells. AM may further contribute to local inflammation by response to alveolar cytokines, not only as phagocytic cells, but as cells capable of calling up and amplifying inflammation by mechanisms consistent with the involvement of XOR.

Hypoxic regulation of MNP XOR may also contribute to the pathogenesis of GLI/Sarcoidosis. Acute inflammatory injury to the lung is typified by pronounced hypoxia, and relative arterial hypoxemia is now considered part of the definition of acute lung injuries (164). Importantly, hypoxia is also a feature of GLI/sarcoidosis that is potentially important in the pathophysiology of granuloma persistence and formation (161-164). XOR hypoxic activation and regulation have been well recognized (209,216,235, and references therein). While XOR activation Hypoxic regulation of MNP XOR may also contribute to the pathogenesis of GLI/Sarcoidosis. Acute inflammatory injury to the lung is typified by pronounced hypoxia, and relative arterial hypoxemia is now considered part of the definition of acute lung injuries (164). Importantly, hypoxia is also a feature of GLI/sarcoidosis that is potentially important in the pathophysiology of granuloma persistence and formation (161-164). XOR hypoxic activation and regulation have been well recognized (209,216,235, and references therein). While XOR activation and regulation have not been studied in myeloid cells, hypoxic regulation has been observed in epithelial cells and fibroblasts. Hypoxic growth induces XOR expression by several fold at pre-translational and post-translational levels as evidenced by the block to induction with actinomycin-D and cyclohexamide (235). Recent evidence indicates that post-translational activation by hypoxia may be transduced by site specific phosphorylation of XOR (209), revealing a key mode of XOR regulation. Conversion from D-form to O-form XOR has not been observed during hypoxic growth in cultured cells (209,216,235). However, the potential for MNP to both induce XOR via hypoxia and to convert XOR into O-form in response to inflammatory cytokines is an important feature of XOR regulation that will be examined in the present work. Hypoxic activation in the MNP population, if accompanied by D-form to O-form conversion, would be expected to further activate MNP ROS generation.

Research into the mechanism of vascular inflammatory injury for much of the preceding several decades has focused on the induction of inflammation and the role played by endothelial cells and endothelial cell XOR, an hypothesis that has been brought into serious question. There are many similarities between general vascular inflammatory injury, ALI, and GLI/sarcoidosis, and it is highly significant that the innate immunological mechanisms of GLI/sarcoidosis bear great similarity to the diffuse inflammatory injuries of ALI, ARDS, COPD, and lung I/R injury (164). In GLI/sarcoidosis, inflammatory cells must be recruited from the vasculature to the lung, adhere, and migrate through the endothelium before taking up residence on the epithelial side and contributing to the formation of the granuloma. Recognition that the inflammatory MNPs express significant levels of O-form XOR and that XOR is an important mediator of general vascular injury and has been linked to GLI/sarcoidosis is likely to be significant inasmuch as oxidative stress generated from XOR could potentiate the inflammatory process at many different levels. The expression and conversion into O-form XOR in the MNPs is not well understood, and certainly understanding the role played by XOR in the MNPs during GLI/sarcoidosis may provide new insights regarding the mechanisms responsible for the pathogenesis of GLI/sarcoidosis and related vascular inflammatory injuries.

Sarcoid-like granulomatous inflammation has been produced in mice by intravenous injection of trehalose-6,6′-dimycolate (TDM), an ether extraction product of Mycobacterium smegmatis (228), or in rats by intravenous injection of heat killed BCG (266,267). Both methods elicit pulmonary granulomas that develop within a few days and resolve by approximately 28 days. Both granulomas exhibit Th-1/MNP infiltration, MNP alveolitis, deposition of extracellular matricies, and fibrosis. While it is presently unknown whether allopurinol would modulate granuloma formation induced with TDM or BCG in rats, human pulmonary sarcoidosis attributed to BCG vaccination was completely regressed following allopurinol treatment (268), indicating a key role for XOR in BCG induced pulmonary granulomatous disease. Several questions emerged from these reports concerning the involvement of XOR in inflammatory lung disease and GLI/sarcoidosis that influence the general clinical utility of allopurinol or influence sensitivity to XOR inhibition. In particular, the cellular environment of GLI/sarcoidosis will depend critically upon the time at which inflammation is assessed and the nature of the inducing stimulus. The temporal order of events will modulate the relative composition of T-cells, MNP, and perhaps other cells (161-164) and this is likely to influence the role played by XOR. Furthermore, the cytokine environment of the inflamed lung is likewise subject to modulation by the nature of the inflammation induced and the time at which inflammation is assessed. For example, pro-inflammatory cytokines like IFN-γ, IL-1, IL-2, or IL-6 may play critical early roles during the induction of inflammation while anti-inflammatory cytokines like IL-10 may play roles later in the course of inflammation (269), and these are likely to affect the state of XOR expression. In addition, at any particular point in the course of inflammation a complex and variable cytokine environment will arise. Since XOR can be activated by several inflammatory cytokines or by phagocytosis its state of activation and contribution to inflammation may be dependent on the specific cytokine signals and combinations that arise during inflammation. For example, the so called “suppressors of cytokine signaling” (SOCS proteins) can be induced by IFN-γ, IL-1, or IL-6 attenuating signals from other cytokines (110-114) which would then be anticipated to influence XOR activation and the course of inflammation as a function of the balance of these different cytokines. Furthermore, our data revealed near complete conversion of XOR from D-form to O-form by the combined action of IL-1 and IFN-γ, and it is reasonable to anticipate that conversion may also be subject to the same complex environment and this is anticipated to influence the contribution of MNP XOR to inflammation as well.

Since XOR has not previously been studied in any experimental model of GLI/sarcoidosis, we will initially characterize GLI/sarcoidosis in rats developed in two ways. The proposed studies will then identify specific cellular and cytokine environments in which XOR is induced and will identify the temporal and cellular response patterns of XOR activation. Finally, we will determine the consequences to the lung of inflammatory XOR activation and will then determine the degree to which inhibition of XOR is protective of lung integrity in rat models of GLI/sarcoidosis.

Characterization of the inflammatory response induced by BCG and TDM in rat models of GLI/sarcoidosis. Allopurinol treatment attenuated GLI/sarcoidosis in humans and attenuated cytokine induced ALI in rats, however, it is not known whether allopurinol would be protective in rat models of GLI/sarcoidosis. Accordingly, we propose to induce GLI/sarcoidosis in rats using two well established protocols (266-268), to characterize the inflammation induced, and to characterize the response of XOR.

Induction of GLI/sarcoidosis in rats. (A) Since we are chiefly interested in the inflammatory response induced by clinically relevant inducers, we will first induce GLI/sarcoidosis in Sprague Dawley rats using heat killed BCG (Mycobacterium bovis strain) since this is a well established means of generating a clinically relevant inflammation in rats (266,267). BCG (Aventis Pharmaceuticals Inc, Bridgewater, N.J.) will be applied by intravenous injection (i.v.) first at a variable sensitizing dose (0.1 to 1.0 mg per rat). Three weeks later rats will be challenged by i.v. injection of a five fold higher dose of heat killed BCG. PCR analysis of a large cohort of sarcoidosis patients revealed that the most common DNA associated with pulmonary sarcoidosis was that of M. tuberculosis (275). Thus, we will utilize BCG derived from M. tuberculosis (Aventis Pharmaceuticals) as the second granuloma inducing agent to be tested. (B) We will next examine alternative granuloma inducing protocols since it is unknown whether pulmonary granuloma formation will show uniform dependence on XOR. TDM, from M. smegmatis, will be applied as described (228) but at doses adjusted to the weight of Sprague Dawley rats. For each of these three agents, we will monitor the inflammatory response over a period from 0 to 28 days and will include four rats at each of six points (0,3,7,14,21,28 days) to establish uniformity of response and the time course of development and regression of the granulomas.

Characterization of the granulomatous inflammatory response. Presently, we employ several methods to characterize pulmonary inflammation that produce concrete evidence of inflammation. First, routine H&E (hematoxylin and eosin) histology will be performed on two rats in each group. To achieve high quality specimens, lungs will be perfused blood free and insufflated with paraformaldehyde fixative with low positive pressure delivered by an infusion pump at 20 mm Hg. This will expand the alveolar space without rupture of the underlying architecture, simultaneously fixing the tissue. These tissues will then be embedded in paraffin, sectioned, and mounted on glass slides. H&E evidence for granuloma formation will include the appearance of organized granuloma, the presence of inflammatory cells in the airway (scaled 0, +to ++++), alveolar swelling, and the degree of perivascular infiltration. Paraffin embedded specimens will be stored at room temperature for additional analyses (for example, XOR immunofluorescence). Since the opportunity to study the involvement of XOR in a systemic response arises following I.V. application of heat killed BCG or TDM, we will also harvest liver, kidney, spleen, brain, heart, and thymus for similar analyses.

BALF will be obtained from two rats in each group, sedimented, and the resulting cells and fluid (PBS) separately collected. We will use the BAL fluid fraction and cell fraction to characterize additional markers of inflammation. Cells will be suspended in PBS, counted for total cells, and an aliquot sedimented on to glass slides and Wright's stained. Differential counts of lymphocytes, MNP, and neutrophils will then be obtained from the stained cytospins. Remaining BALF cells will be sedimented and frozen for subsequent biochemical analyses. Protein concentration will be determined from the BAL fluid fraction and the remaining fluid frozen for additional biochemical analyses.

XOR activation and conversion in vivo. We will quantitate and compare both lung tissue specimens and BALF cell fractions (lymphocytes, MNP, PMN) for XOR in several ways. D-form and O-form activity will be assayed biochemically from total lung tissue extracts using our standard spectroscopic assay for the formation of uric acid (217,265). BALF cells will be purified on Percoll gradients which produce lymphocytes, MNP, and PMN populations of well over 95% purity. These fractions will be assayed as well for D-form and O-form XOR activity. Each of these samples will also be assayed by Western-immunoblot to corroborate activation of XOR in the lung and to determine the extent, if any, to which XOR has been proteolyzed. We imagine that the conversion of XOR into O-form may be dependent upon the specific granulomatous inducer and by the time at which XOR is assayed. Verification that XOR has been converted reversibly into O-form will be established by reduction with DTT and re-chromatography on Sephadex G25 followed by re-assay of D-form and O-form content. Our previous data revealed dramatic activation in the MNP population and that 80% of the lung and MNP XOR was converted to O-form by cytokine induced ALI, however, it is unknown whether this degree of activation and conversion will arise in granulomatous injury or when granuloma is induced by different agents.

XOR localization. FACS analysis revealed activation and expression of XOR in the immature and differentiating MNPs following cytokine insufflation (265). However, XOR activation may occur in lung tissue cells, T-cells, in several different MNP populations, activated macrophages, antigen presenting macrophages (dendritic cells), or interstitial macrophages. Tissue samples that had been saved as paraffin embedded specimens also will be used for XOR co-immunofluorescence analysis to identify specific cells expressing XOR in vivo during GLI/sarcoidosis induced by BCG or TDM. We have utilized the MNP specific antibody to ED1 (276) to detect mature alveolar macrophages recovered in the BALF, and this antisera (FITC conjugated) will be used in co-immunofluorescence with XOR (Rhodamine) to confirm localization in the alveolar macrophage. Antisera to ED2, which specifically identifies the interstitial macrophage, will be used in a similar fashion to determine if XOR is activated in this population. CD68 is a nonspecific MNP surface marker (277) that will identify MNP in the rat granuloma and its use in co-immunofluorescence with anti-XOR antisera will serve as a strong evidence for colocalization of XOR to granulomatous MNP, while co-immunofluorescence with OX6 will identify expression specifically in activated rat macrophages (278). CD11b antisera will allow us to identify co-immunolocalization in the immature MNP and PMN cells which can be readily differentiated in situ morphologically. Recent evidence indicates a critical role for CD40+ macrophages in chronic lung inflammation that is consistent with the involvement of immunoreactive T-cells (279-281). Accordingly, we will also conduct co-immunofluorescence with anti-CD40 and anti-XOR antisera to determine whether XOR is expressed in CD40+ macrophages. While there is presently no evidence to suggest expression of XOR in any T-cell population, similar analyses performed with CD4 or CD8 antisera may yield unexpected observations critical to the present analyses. These experiments will allow us to further define the specific T-cell or MNP cell population that expresses XOR in vivo during experimental GLI/sarcoidosis in a defined rat model.

We will also collect each cell fraction from percoll gradient purified BALF cells, or prior to percoll fractionation, by cytospin onto microscope slides and detect XOR by immunofluorescence. Co-immunofluorescence analyses with the above MNP surface markers should allow us to assign XOR expression to specific cells of the MNP (or other) lineage. We have also generated compelling data using FACS analysis of BALF cells recovered from lungs of cytokine insufflated rats, and co-immunofluorescence analyses with antisera for XOR and various MNP surface markers will add substantial verification to the data generated here. Co-immunofluorescence with FACS analysis will simultaneously quantitate the number of XOR expressing MNP cells, the specific population of MNP in which XOR is expressed, and compile data on general cell shape.

The protective effect of XOR inhibition will be tested in two ways in rat models of GLI/sarcoidosis.

XOR inhibition. XOR is routinely inhibited in experimental animals by supplementing diets with allopurinol or feeding them on rodent chow that has been depleted of molybdenum and enriched in tungsten (282). Our data (265) show the results of using both strategies. In the present experiments we will inhibit XOR initially with allopurinol since the period required to achieve XOR inhibition is short, one week, compared to that needed for tungsten inhibition, six weeks. As a soluble competitive inhibitor of XOR, allopurinol has the disadvantage that XOR itself cannot be directly assayed following inhibition. Furthermore, some concern exists that allopurinol may act to scavenge ROS during inflammation, independent of its effects on XOR. Thus, where a role for XOR has been indicated, we will verify these results using molybdenum deficient, tungsten enriched diets that have been applied for six weeks. Corroboration with tungsten inhibition will constitute strong evidence for the involvement of XOR catalytic activity since tungsten substituted XOR is inactive (282).

Evaluation of inflammation. Several elements of the granulomatous response can reflect the potentially beneficial or deleterious affects of XOR inhibition. XOR inhibition attenuated markers of inflammation induced by cytokine insufflation, including an improved histological picture, decrement in alveolar cell apoptosis, decrement in MNP oxidative stress, and decrement in the net number of inflammatory cells recovered in the BALF. However, XOR may contribute to the improved efficiency of macrophage phagocytosis and thereby provide a benefit to the inflammatory response. Our assessment of inflammation induced following XOR inhibition will assess both the advantages and disadvantages of XOR inhibition by quantitating several markers of inflammation in both the lung tissue and the BALF cell fraction.

Lung tissue specimens will be obtained from rats exposed to control diets, allopurinol, or tungsten supplemented diets and subsequently treated to induce pulmonary granuloma using each of the different strategies described above. We will assay these specimens in several ways. Routine H&E histology will be performed to assess the overall status of the lung and characteristics of granuloma formation and regression over the 28 day period. Immunofluorescent assay for XOR antigen will be performed as well. While neither inhibitor strategy will itself block expression of XOR immuno-reactive protein, reduction in recruitment of inflammatory cells will reduce the abundance of XOR. Quantitative morphometric analysis of nitrotyrosine and nitrotyrosine western immunoblot will be performed on these same samples.

Cells will be obtained from the BALF, Wright's stained, and the differential count of lymphocytes, MNP, PMN, and total cell count will be obtained. This will establish the degree to which the net inflammatory response has been affected by XOR inhibition. Since XOR inhibition could adversely affect macrophage phagocytosis, we will also quantitate the number of dead or dying cells recovered in the BALF using propidium iodide staining. An excess of unphagocytized cells in the BALF that can be attributed to allopurinol inhibition may reflect an unanticipated consequence of XOR inhibition, something that may become more evident in later stages of the inflammatory process. The cell free BALF fluid fraction will be assayed for net protein concentration as an additional marker of ongoing lung inflammation. Additional consequences of XOR inhibition will be taken up below.

D-form and O-form XOR activity and immunoreactive protein (western blot) will be assayed from lung tissues and BALF cell fractions as described above for each of the granulomatous inducing strategies in control and tungsten inhibited rats to confirm suppression of XOR activity and possibly protein abundance.

Timing of XOR inhibition. We presently imagine that pretreating rats with XOR inhibiting diets will most effectively reveal a role for XOR in pulmonary granulomatous injury, however, this strategy is of limited clinical value. For this reason, we will determine the effect of inhibiting XOR with allopurinol at the time granuloma is induced (not available for tungsten inhibition). Since it is uncertain that concomitant inhibition can achieve useful attenuation of inflammation, we will initially monitor lungs by H&E histology and BALF cell count. In the event that concomitant inhibition does affect the granulomatous response over the extended 28 day time course, additional markers of inflammation will be assessed as well.

These experiments will determine whether XOR contributes to experimentally induced granulomatous inflammation in rats. While XOR activation in macrophages improves the efficiency of macrophage phagocytosis, inhibition of XOR can also attenuate inflammation and this may reflect the protective effect of allopurinol in numerous inflammatory disorders. Thus, the utility of XOR inhibition needs to be evaluated in the context of its potentially beneficial affects and its potentially deleterious affects. We have proposed to use allopurinol as the drug of choice for inhibiting XOR in vivo because it is well accepted for use in humans, has minimal deleterious side affects, and is relatively rapid in action. While potentially useful data could be obtained using XOR knock out mice, this system may ultimately be less conclusive than that obtained by allopurinol inhibition. Double XOR knock outs (XOR^{-/-}) die within several weeks of birth, revealing some unanticipated role of XOR in development (283). Heterozygous knock-outs (XOR^{+/-}) are unlikely to have sufficiently reduced levels of XOR to draw any conclusive inferences. We propose to confirm data obtained with allopurinol inhibition using tungsten inhibition, a generally well accepted method. While several pharmacological inhibitors of XOR have been developed recently, these are considered to be presently inferior to allopurinol because little is known of their alternative actions or free radical scavenging capability. Nonetheless, sensitivity of experimentally induced GLI/sarcoidosis to allopurinol or tungsten inhibition may suggest the merit of determining the sensitivity to these alternative, and potentially more rapidly acting, XOR inhibitors. Overall, the experiments in this specific aim will determine whether XOR activation is important for granulomatous lung inflammation induced by different strategies. They will establish whether XOR activation and conversion are consistent features of MNP cell infiltration, and they will establish the degree to which MNP XOR is specifically important to granulomatous lung inflammation or whether additional sites of activation (endothelial cells) arise as well.

The mechanism by which XOR activation in the MNP cells contributes to the inflammatory process in a rat model of GLI/sarcoidosis. Since the primary contribution of XOR to inflammation is thought to be that of ROS generation, we will first characterize ROS generation from XOR in MNP cells in vivo and in vitro in the rat models of GLI/sarcoidosis. We will then characterize the response of the whole lung and the MNP cells to ROS generation by XOR in the MNP cells in vitro and in vivo.

Characterization of ROS generation from XOR in MNP cells in vitro and in vivo. We imagine that the critical role played by XOR in pulmonary granulomatous inflammation will be as a source of ROS that serves to promote the inflammatory process or to delay resolution of inflammation. The existence of an ongoing oxidative stress will be determined in several ways that allow us to infer a contribution by XOR.

In vivo generation of oxidative stress resulting from XOR activation. Our previous data demonstrated allopurinol inhibitable nitrotyrosine protein modification in MNP cells in cytokine induced ALI. Since nitrotyrosine formation can reflect the formation of superoxide radical by XOR, it is important to determine whether MNP XOR also generates nitrotyrosine in the rat models of GLI/sarcoidosis. We will use the anti-nitrotyrosine immune recognition analyses to address several questions surrounding the oxidative stress generated in vivo by XOR activation in the MNP. Tissues will be stained from sham or granuloma induced lungs using Hoescht (to stain nuclei), wheat germ agglutinin (to stain tissue architecture), and anti-nitrotyrosine (to stain protein derived nitrotyrosine) as shown previously (265). Nitrosylation of tyrosine proceeds through a well defined pathway that is dependent upon the formation of superoxide anion (the ROS) and NO to form ONOO, the nitrating species. This assay has the great advantage of being able to identify sources of ROS on paraffin embedded, archived, tissue sections since the nitration product is quite stable. Tissue slices derived from the same specimen blocks used for XOR localization will also be used in co-localization analyses using peroxidase conjugated anti-nitrotyrosine antisera and FITC conjugated anti-XOR antisera. Since approximately 14% of lung MNP cells exhibit auto-fluorescence, we have shifted our analyses to a peroxidase/NBT immunoprecipitation assay (265). Quantitative morphometric analysis of cytokine induced ALI revealed that 84% of the alveolar MNP became nitrotyrosine positive, and this was restored to background by allopurinol treatment (265), and peroxidase coupled detection is anticipated to produce even more unambiguous data. These data will allow us to identify and quantitate the specific MNP cell population expressing XOR and to confirm the presence of an ongoing oxidative stress in them. Furthermore, particular attention will be paid to the site in the inflamed lung where these cells derive. For example, it is expected that early in the course of granuloma formation, MNP may exist in the tissue parenchyma or alveolar airspace prior to granuloma formation, and these cells may already exhibit oxidative stress. We presently imagine the granuloma itself to exhibit variable oxidative stress throughout the 28 day time course evaluated.

We will obtain independent corroboration that nitrotyrosine formation has been stimulated by the induction of GLI/sarcoidosis using Western immunoblot analysis of whole lung extracts and of specific cell fractions as described above. We will again substitute the secondary antibodies with peroxidase conjugated antisera and use them in peroxidase/luciferin/luciferase light generation. These data will allow us not only to confirm the formation of nitrotyrosine during the time course of granuloma formation and resolution, but will offer an opportunity to quantitate the nitrotyrosine by image analysis. We will also perform these analyses on cells recovered in the BALF since this may indicate a relatively simple procedure for rapidly obtaining comparable data. These experiments are effective only for cells freely available in the airway, and would therefore provide no information on the interstitial MNP or MNP within the granuloma. Nonetheless, they may indicate an ongoing oxidative stress in MNP prior to entering the granuloma.

To confirm the role of XOR in the genesis of oxidative stress in vivo during granulomatous inflammation, these analyses will also be performed in rats that have been exposed to XOR inhibiting diets. The combination of allopurinol and tungsten inhibitor diets will allow us to assign an unambiguous role to XOR. Clearly, the lung has numerous sources of ROS that may be activated during experimental GLI/sarcoidosis. Failure to block nitro-tyrosine formation with tungsten or allopurinol feeding would indicate activation of ROS generators other than XOR (NADPH oxidase, myeloperoxidase, cyclooxygenase), however, our data reveal the striking contribution to MNP ROS generation by XOR following cytokine induced inflammation, and this is likely to arise in granulomatous inflammation as well. Evidence for XOR derived nitrotyrosine in the rat models of granulomatous inflammation will indicate the importance of conducting corroborative assays for XOR derived carbonyl proteins (169) or lipid peroxidation products (178).

In vitro generation of oxidative stress by XOR activation in MNP. We have quantitated hydrogen peroxide release from whole BALF cells in a Lumistar Luminometer using a coupled peroxidase/luciferin/luciferase assay and have quantitated superoxide release using a spectroscopic assay for cytochrome c reduction (207). We will quantitate the release of hydrogen peroxide and superoxide from BALF cells before and after stimulation with the XOR substrates, xanthine and or hypoxanthine, to confirm that XOR derived ROS is indeed released from these cells. Cells from the same experiment will be sedimented on to glass slides (cytospin), fixed, and stained with Alexa 594 conjugated anti-nitrotyrosine antisera (265). Assay of cells from XOR inhibited rats will allow us confirm XOR, specifically in the BALF cells, as a source of hydrogen peroxide, superoxide anion, and protein nitration.

We will perform comparable experiments on inflammatory cells isolated from induced and inhibited whole lungs. Lungs will be perfused blood free, minced on ice with scissors, and exposed to collagenase for two hours. After straining the tissue debris, MNP will be adhered to plastic dishes for one hour in serum rich cell culture medium (265). Adherent cells will be washed and assayed as above. As in the preceeding section, our analyses will be performed on rats exposed to control and XOR inhibiting diets prior to induction of granuloma.

Contribution of XOR to apoptosis in the lung during granulomatous inflammation. Our previous data revealed that alveolar cell apoptosis was stimulated 13 fold by cytokine induced ALI, and that prior inhibition of XOR with allopurinol attenuated cytokine induced alveolar apoptosis. However, these analyses did not assess apoptosis within the inflammatory cells themselves. It has been postulated that reduced apoptosis in the granuloma is responsible for persistence of the granuloma, while resolution is associated by increased apoptosis in the granuloma (284). Excess levels of the apoptosis inducers Fas/FasL are found in sarcoid granuloma (285-287), but are associated with reduced apoptosis in the granuloma (288). This paradox has been explained by the induction of p21^{Waf1} by IFN-γ in the inflammatory cells (289) which renders them resistant to Fas induced apoptosis (288). Since allopurinol promotes the resolution of established granuloma (182-186), we will assess the level of apoptosis in the rat models of granulomatous inflammation over the course of 28 days from control and allopurinol fed rats. Our assays for in vivo apoptosis will constitute histochemical TUNEL stain and immunocytochemical analysis of caspase 3 activation as previously described (265). Particular attention will be paid to the localization of TUNEL/caspase signals, since it is possible that localized alveolar apoptosis itself may promote the inflammatory process. It can be anticipated that a complex picture will unfold throughout this time course, and the effect of allopurinol on the course and localization of apoptosis is likely to yield highly valuable information.

Considerations. We have proposed three kinds of analyses for XOR dependent oxidative stress. Nitrotyrosine analysis will establish the generation of superoxide in specific cell populations in vivo using lung tissue slices and cells recovered in the BALF following experimentally induced granuloma in rats fed control or XOR inhibited diets. They will also allow us to infer the presence of ROS generating XOR in non-inflammatory cells of the lung. Since very specific control reactions can be conducted for nitrotyrosine (290,291), these reactions are preferred over other in situ methods such as DCFH (difluorofluroescein) oxidation. Furthermore quantitative morphometric analyses will be used to quantitate the increase in nitrotyrosine staining, and these data will also be corroborated by Western immunoblot. The formation of hydrogen peroxide and superoxide in whole cells supplemented with xanthine or hypoxanthine will establish the capacity of XOR to form ROS in the BALF cells or interstitial cells obtained from collagenase treated lung homogenates following granuloma induction, and these data will be confirmed using cells from XOR inhibited rats. We have used other methods of ROS detection in live cells, including DCFH fluorescence, and while these may ultimately prove useful, they are subject to significant artifactual conversion of the DCFH and difficult background problems that generate highly ambiguous results. The proposed studies will be pertinent to our understanding of granulomatous inflammation because suppression of XOR activity in an ongoing inflammatory process has the capacity to attenuate inflammation, oxidative stress, and alveolar cell apoptosis. The mechanism by which XOR contributes to these different processes in sarcoidosis may suggest alternative modes of intervention or specific strategies related to the timing of intervention.

Characterization of the response of the MNP cells to ROS generation by XOR in the MNP cells in vitro and in vivo. Our data have indicated that expression of XOR within the inflammatory MNP cells can modulate cytokine induced ALI. We imagine that the fundamental biology of the MNP cells will be substantially the same in granulomatous inflammation, therefore, it is likely that expression of XOR may also modulate the behavior of MNP in GLI/sarcoidosis.

Cytokine synthesis and release. Inhibition of XOR specifically within the MNP attenuated the recruitment of inflammatory cells to the lung and resulted in the recovery of immature MNP in the BALF. We presently imagine that XOR derived ROS signaling within the MNP cells may stimulate expression of chemotactic factors by a process that is sensitive to allopurinol inhibition. ROS sensitive cytokines and chemokines that may be affected by XOR derived ROS include MCP-1, IL-1, IL-8, IL-6 (292). Several strategies are presently available for determining cytokine levels in BALF. We have used cytokine array immunoblots in which anti-cytokine antibodies have been bound to nitrocellulose. These have been reacted to cell free lavage fluid during IL-1 and IFN-γ induced inflammation and have revealed increases in BALF of IL-4, IL-8, TGF-β, and MCP-1 at 24 hours post-insufflation. While somewhat limited in sensitivity, the blots will detect nanogram levels of cytokine and are thus adequate to detect most changes in cytokine level in the BALF fluid fraction.

Thus, we will obtain BALF from rats subjected to granulomatous inflammation and collect the inflammatory cell fraction and the cell free fluid fraction. Anti-cytokine antibody array blots (RayBiotech custom cytokine array) will be used to assay up to 20 cytokines and chemokines simultaneously. The comparison of sham treated BALF to experimentally induced granuloma with and without allopurinol will allow us to identify a contribution of XOR to cytokine or chemokine alteration during inflammation. Cytokine changes found to correlate with XOR activation will be corroborated with cytokine specific ELISA for which peptide specific competition analysis will provide definitive evidence of cytokine elevation or modulation. In addition, we will assay the differentiation promoting BMP proteins (bone morphogenetic proteins-2,3,4,5,6,7) by Western immunoblot analysis of BALF, since these protein may have significant effects on maturation of lung MNP and may be sensitive to XOR derived ROS (250). Direct assay of the cell free BALF is desirable since many of the important cytokine mediators can be derived from the epithelial cells or other alveolar cells.

We will also use the BALF cell fraction from the same rats to isolate RNA for use in cytokine array RNA analyses by RNA protection analysis (RPA). These experiments will allow us to compare expression of specific cytokines or chemokines from the same rats in which cytokines and chemokines have been assayed at the level of protein expression, but have the additional advantage of being conducted on the inflammatory cells themselves. Corroboration that an RT-PCR signal has indeed been derived from the MNP cells will be obtained by Northern blot of specific cytokines or chemokines using Percoll gradient purified MNP. In combination, these data will allow us to assign a specific role of MNP derived XOR in the stimulation of cytokines or chemokines in an in vivo model of GLI/sarcoidosis.

Adhesion molecule expression. XOR derived ROS specifically in the MNP cells may contribute to adhesion molecule activation or expression and thereby affect the recovery of inflammatory cells in the BALF (242-244). Thus, we will obtain circulating monocytes and BALF cells from rats subjected to experimentally induced granuloma in the presence and absence of prior XOR inhibition. Cells will assayed for expression of ICAM-1, VLA-3, VLA-4, and other integrin molecules by FACS analysis and Western immunoblot analysis since many of these adhesion molecules may also be regulated by ROS sensitive transcription factors.

Transcription factor activation. A specific consequence of ROS generation from MNP XOR expression is expected to be activation of ROS sensitive transcription factors such as NF-κB, AP-1, or STAT factors which may subsequently promote chemokine or adhesion molecule expression. We imagine this to be a necessary prelude for activation of chemotactic factors. However, the activation of such broadly active transcription factors would suggest that many genes could be subject to alteration in activation state with consequences potentially greater than the activation of specific cytokines or chemokines. Accordingly, we will obtain inflammatory cells from the BALF of experimentally induced GLI/sarcoidosis, isolate nuclei and nuclear proteins from these cells, and use the nuclear protein fraction to assay activation of NF-κB, AP-1, and STAT1, STAT3, and STAT5 using a combination of electrophoretic mobility shift assay (EMSA) and Western blot analyses. The contrast between experimental groups in which XOR has been inhibited with allopurinol will allow us to assign a role for MNP XOR in the activation of selected ROS sensitive transcription factors likely to regulate many aspects of the evolving inflammatory process, including stimulation of chemokine expression.

Participation of XOR in MNP differentiation and maturation. Our data revealed that inhibition of XOR specifically within the MNP cells attenuated the recruitment of inflammatory cells to the lung and revealed changes in the MNP population itself (265), suggesting that XOR may exert unanticipated affects on MNP development or maturation. To determine whether XOR contributes to maturation of MNP, BALF cells will be obtained from rats following induction of granulomatous inflammation in rats fed control or allopurinol diets. These cells will be analyzed in several ways to characterize the state and condition of the cells. Aliquots of recovered cells will be stained and quantitated to determine the differential cell count. Additional aliquots will then be analyzed by FACS analysis using FITC conjugated antisera to CD11b, ED1, and ED2 to determine the state of maturation (immature, CD11b+/ED1−/ED2−; mature CD11b−/ED1+ or ED2+). Cells and BALF will also be assayed for expression of cytokines and chemokines as described above since cytokine expression profile is an important characteristic of MNP cells that changes during differentiation and maturation. Cytospin or Western immunoblot analysis of the differentiation determinants, Id-1 and Id-2, from control and allopurinol inhibited BALF cells will yield important insight into the mechanism by which XOR may influence lung MNP differentiation itself since these proteins are key determinants of leukocyte and lymphocyte development (293,294). We envision a mechanism by which XOR or XOR derived ROS may promote MNP maturation that would itself modulate cytokine synthesis and release. Blockade of this process with allopurinol may then result in the accumulation of the differentiation inhibitor, Id-2 (below), and arrest of MNP development, perhaps affecting the capacity for expression of cytokines or chemokines.

Western immunoblot analysis of the differential inhibitor, Id-2, from BALF cells obtained 24 hours following cytokine insufflation in allopurinol (A) or control (C) fed rats. These data show XOR dependent regulations of MNP Id-2 levels in cytokin induced ALI.

In parallel experiments, we will determine the effect of allopurinol inhibition on MNP differentiation and viability using BALF cells obtained from rats that have been exposed to BCG/TDM or sham injection after feeding on control or allopurinol supplemented diets. These cells will be assayed as above to determine the degree of viability and apoptosis, the state of cytokine expression, surface markers of differentiation, and expression of Id determinants of differentiation. The data obtained from these experiments will allow us to determine whether XOR inhibition within the MNP cells has increased the rate of alveolar macrophage cell death or has altered the state of maturation of MNP newly recruited to the lung.

Use of animals. We will use approximately 200 male and female Sprague Dawley rats each year. Rats will be given TDM or heat killed BCG intravenously to induce granulomatous inflammation. They will first receive a sensitizing dose as described and will then be stimulated intravenously three weeks later with a five fold higher dose of BCG. TDM will be administered as a single intravenous dose. At various times thereafter rats will be sacrificed. Lungs and BALF cells will be recovered following vascular perfusion with saline and both will be examined in vitro. We will quantitate several parameters of lung granulomatous inflammation from harvested lungs. Rats will used in four experimental groups: sham injected controls, induced rats, and rats induced with BCG or TDM that had been previously fed on XOR inhibiting diets. Diets will consist of allopurinol substituted feed or feed depleted in molybdenum and supplemented with tungsten as described (193,206,265). This procedure has been approved by the Webb-Waring Institute's AAALAC accredited animal care facility.

Veterinary care. All rats are monitored daily for illness or evidence of distress by a certified laboratory animal technician who is under the supervision of a laboratory animal veterinarian. Sick rats will either be euthanized immediately or provided with appropriate medical care. The veterinarian is on site during the daytime and available at other times by pager.

Pain control. Rats will be treated with inflammatory inducers by intravenous, tail-vein, injection recovered in cages under constant supervision. Sample collection will be done as a terminal procedure with rats under deep anesthesia. Rats will be given sodium pentobarbital and the chest will be opened only after the rat is totally unresponsive to any stimulus. Thus, rats should not experience any suffering other than the prick of a 25 gauge needle to administer the inducer or the anesthesia. Euthanasia of all rats will follow AVMA approved methods.

Other Modulators. Compounds that can be added as modulators are Diphenyleneiodonium dichloride to block nitric oxide synthesis; and glutathione, glutathione precursors and and dimethylthiourea (DMTU)to inhibit D-form to O-form conversion.

All cited patents, patent applications and publications and other documents cited in this application are herein incorporated by reference in their entirety.

### LITERATURE CITED

1. Prince, J, E., Kheradmand, F., and Corry, D. B.: Immunologic lung disease. J. Allergy Clin. Immunol. 111: S613-S623, 2003.

2. Ware, L. B. and Matthay, M. A.: The acute respiratory distress syndrome. New England J. Med. 342: 1334-1349, 2000.

3. Matthay, M. A., Zimmerman, G. A., Esmon, C., Bhattacharya, J., Coller, B., et al.: Future research directions in acute lung injury. Summary of a NHLBI working group. Am. J. Respir. Crit. Care Med. 167: 1027-1035, 2003.

4. MacNee, W.: Oxidative stress and lung inflammation in airway disease. Eur. J. Pharm. 429: 195-207, 2001.

5. Newman, L. S., Rose, C. S. and Maier, L. A.: Sarcoidosis. New England J Med 336: 1224-1234, 1997.

6. Agostini, C, Facco, M., Chilosi, M., Semenzato, G.: Alveolar macrophage-T cell interactions during Th-1 type sarcoid inflammation. Microsc. Res. Tech. 53: 278-287, 2001.

7. Landmesser, U., Spiekermann, S., Dikalov, S., Tatge, H., et al.: Vascular oxidative stress and endothelial cell dysfunction in patients with chronic heart failure. Role of xanthine oxidase and extracellular superoxide dismutase. Circulation 106: 3073-3078, 2002.

8. Farquharson, C. A. J., et al.: Allopurinol improves endothelial dysfunction in chronic heart failure. Circulation 106:221-226, 2002.

9. Doehner, W., et al.: Effects of xanthine oxidase inhibition with allopurinol on endothelial function and peripheral blood flow in hyperuricemic patients with chronic heart failure. Circulation 105:2619-2624, 2002.

10. Cappola, T. P., et al.: Allopurinol improves myocardial efficiency in patients with idiopathic dilated cardiomyopathy. Circulation 104:2407-2411, 2001.

11. Clancy, R. R., McGaurn, S. A., Goin, J. E., Hirtz, D. G., et al.: Allopurinol neurocardiac protection trial in infants undergoing heart surgery using deep hypothermic circulatory arrest. Pediatrics 108: 61-70, 2001.

12. Weimert, N. A., Tanke, W. F., and Sims, J. J.: Allopurinol as a cardioprotectant during coronary artery bypass graft surgery. Ann. Pharmacotherapy 37: 1708-1711, 2003.

13. Garattini, E., Mendel, R., Romao, M. J., Wright, R. M., and Terao, M.: Mammalian molybdo-flavoenzymes, an expanding family of proteins: structure, genetics, regulation, function, and pathophysiology. Biochem. J. 372: 15-32, 2003.

14. Desco, M. C., et al.: Xanthine oxidase is involved in free radical production in type 1 diabetes: protection by allopurinol. Diabetes51:1118-1124, 2002.

15. Butler, R., et al.: Allopurinol normalizes endothelial dysfinction in type 2 diabetes with mild hypertension. Hypertension 35:746-751, 2000.

16. Aliciguzel, Y., Ozen, I., Aslan, M., and Karayacin, U.: Activities of xanthine oxidoreductase and antioxidant enzymes in different tissues of diabetic rats. J., Lab. Clin. Med. 142:172-177, 2003.

17. Matsumoto, S., Koshiishi, I., Inoguchi, T., Nawata, H., and Utsumi, H.: Confirmation of superoxide generation via xanthine oxidase in streptozocin induced diabetic mice. Free Rad. Res. 37: 767-772, 2003.

18. Matsumura, N., Ochi, K., Ichimura, M., Mizushima, T., et al.: Study on free radicals and pancreatic fibrosis. Pancreatic fibrosis induced by repeated injections of superoxide dismutase inhibitor. Pancreas 22: 53-57, 2001.

19. Czako, L., Takacs, T., Varga, I., Tiszlavicz, L., et al: Involvement of oxygen derived free radical in L-arginine induced pancreatitis. Dig. Dis. Sci. 43: 1770-1777, 1998.

20. Kruidenier, L., Kuiper, I., Lamers, C. B., and Verspaget, H. W.: Intestinal oxidative damage in inflammatory bowel disease: semi-quantification, localization, and association with mucosal antioxidants. J. Pathol. 201: 28-36, 2003.

21. Riaz, A. A., Schramm, R., Sato, T., Menger, M. D., et al.: Oxygen radical dependent expression of CXC chemokines regulate ischemia/reperfusion induced leukocyte adhesion in the mouse colon. Free Rad. Biol. Med. 35: 782-789, 2003.

22. Riaz, A. A., Wan, M. X., Schafer, T., Dawson, P., et al.: Allopurinol and superoxide dismutase protect against leukocyte endothelium interaction in a novel model of colonic ischemia/reperfusion. Br. J. Surgery 89: 1572-1580, 2002.

23. Xiang, L., Klintman, D., and Thorlacius, H.: Allopurinol inhibits CXC chemokine espression and leukocyte adhesion in endotoxemic liver injury. Inflamm. Res. 52: 353-358, 2003.

24. Ohta, Y., Kobayashi, T., and Ishiguro, I.: Participation of xanthine oxidase system and neutrophils in development of acute gastric mucosal lesions in rats with a single treatment of compound 48/80, a mast cell degranulator. Dig. Dis. Sci. 44: 1865-1874, 1999.

25. Sekundo, W. and Augustin, A. J.: Difference in composition of inflammatory cell infiltrate in lens induced uveitis under therapy with allopurinol or steroids. Eur. J. Ophthalmol. 11: 264-268, 2001.

26. Sekundo, W. and Augustin, A. J., Strempel, I: Topical allopurinol or corticosteroids and acetylcysteine in the early treatment of experimental corneal alkali burns: a pilot study. Eur. J. Ophthalmol. 12: 366-372, 2002.

27. Grus, F. H., Augustin, A. J., Loeffer, K., Lutz, J., and Pfeiffer, N.: Immunological effects of allopurinol in the treatment of experimental autoimmune uveitis after onset of the disease. Eur. J. Ophthalmol. 13: 185-191, 2003.

28. Quinlan, G. J., Lamb, N. J., Tilley, R., Evans, T. W., and Gutteridge, J.M.C.: Plasma hypoxanthine levels in ARDS: implications for oxidative stress, morbidity, and mortality. Am. J. Resp. Crit. Care Med. 155: 479-484, 1997.

29. Grum, C. M., R. A. Ragsdale, L. H. Ketai, and R. H. Simon.: Plasma xanthine oxidase activity in patients with adult respiratory distress syndrome. J. Crit. Care 2:22-27, 1987.

30. Chinnaiyan, A. M., Huber-Lang, M., Kumar-Sinha, C., Barrette, T. R. et al.: Molecular signatures of sepsis. Multiorgan gene expression profiles of systemic inflammation. Am. J. Path. 159: 1199-1209, 2001.

31. Pinamonti, S., Muzzoli, M., Chicca, M. C., Papi, A., Ravenna, F., Fabbri, L. M., and Ciaccia, A.: Xanthine oxidase activity in bronchoalveolar lavage fluid from patients with chronic obstructive pulmonary disease. Free Rad Biol Med 21: 147-155, 1996.

32. Pinamonti, S., Leis, M., Barbieri, A., Leoni, D., Muzzoli, M., Sostero, S., Chicca, M. C., Carrieri, A., Ravenna, F., Fabbri, L. M., and Ciaccia, A.: Detection of xanthine oxidase activity products by EPR and HPLC in bronchoalveolar lavage fluid from patients with chronic obstructive pulmonary disease. Free Rad. Biol. Med. 25: 771-779, 1998.

33. Heunks, L. M., Vina, J., Herwaarden, C. L., Folgering, H.T., Gimeno, A., and Dekhuijzen, P. P. N.: Xanthine oxidase is involved in exercise induced oxidative stress in chronic obstructive pulmonary disease. Am. J. Phyisol. 277: R1697-1704, 1999.

34. Ichinose, M., Sugiura, H., Yamagata, S., Koarai, A., et al.: Xanthine oxidase inhibition reduces reactive nitrogen species production in COPD airways. Eur. Resp. J. 22: 457-461, 2003.

35. Anderson, B. O., Moore, E. E., Moore, F. A., Leff, J. A., Terada, L. S., Harken, A. H., and Repine, J. E.: Hypovolemic shock promotes neutrophil sequestration in lungs by a xanthine oxidase related mechanism. J. Appl. Physiol. 71: 1862-1865, 1991.

36. Shenkar, R. and Abraham, E.: Plasma from hemorrhaged mice activates CREB and increases cytokine expression in lung mononuclear cells through a xanthine oxidase-dependent mechanism. Am. J. Respir. Cell Mol. Biol. 14:198-206, 1996.

37. Modelska, K., Matthay, M. A., Brown, L. A. S., Deutch, E., Lu, L.N., and Pittet, J. F.: Inhibition of B-adrenergic-dependent alveolar epithelial clearance by oxidant mechanisms after hemorrhagic shock. Am. J. Physiol. 276: L844-L857, 1999.

38. Adkins, W. K. and Taylor, A. E.: Role of xanthine oxidase and neutrophils in ischemia reperfusion injury in rabbit lung. J. Appl. Physiol. 69: 2012-2018, 1990.

39. Shibata, K., Cregg, N., Engelberts, D., Takeuchi, A., Fedorko, L., and Kavanagh, B. P.: Hypercapnic acidosis may attenuate acute lung injury by inhibition of endogenous xanthine oxidase. Am. J. Resp. Crit. Care Med. 158: 1578-1584, 1998.

40. Wright, R. M., Ginger, L. A., Kosila, N., Elkins, N. E., Essary, B., McManaman, J. L., and Repine, J. E.: Mononuclear phagocyte xanthine oxidoreductase contributes to cytokine induced acute lung injury. Am. J. Respir. Cell Mol. Biol. 30: 1-12, 2003.

41. Pfau, A., Stolz, W., Karrer, S., Szeimies, R. M., and Landthaler, M.: Allopurinol treatment of cutaneous sarcoidosis. Hautarzt 49: 216-218, 1998.

42. Okamoto, H., Mizuno, K., and Horio, T.: Monocyte derived multinucleated giant cells and sarcoidosis. J. Dermatol. Sci. 31: 119-128, 2003.

43. Mizuno, K., Okarnoto, H., and Horio, T.: Inhibitory influences of xanthine oxidase inhibitor and ACE inhibitor on multinucleate giant cell formation from monocytes by down regulation of adhesion molecules and purinergic receptors. Br. J. Dermatol. (In press).

44. Antony, F., and Layton, A. M.: A case of cutaneous acral sarcoidosis with response to allopurinol. Br. J. Dermatol. 142: 1052-1053, 2000.

45. El-Euch, D., Mokni, M., Trojjet, S., Khouaja, A., and Ben-Osman, A.: Sarcoidosis in a child treated successfully with allopurinol. Br. J. Dermatol. 140: 1184-1185, 1999.

46. Guthikonda, S., Sinkey, C., Barenz, T., and Haynes, W. G.: Xanthine oxidase inhibition reverses endothelial dysfunction in heavy smokers. Circulation 107: 416-421, 2003.

47. Wang, H. Y., Ma, L., Li, Y., and Cho, C. H.: Exposure to cigarette smoke increases apoptosis in the rat gastric mucosa through a reactive oxygen species mediated and P53 independent pathway. Free Rad. Biol. Med. 28: 1125-1131, 2000.

48. Ma, L., Chow, J. Y., and Cho, C. H.: Mechanistic study of adverse actions of cigarette smoke exposure on acetic acid induced gastric ulceration in rats. Life Sci. 62: 257-266, 1998.

49. Akgur, F. M., Brown, M. F., Zibari, G. B., McDonald, J. C., et al.: Role of superoxide in hemorrhagic shock induced P-selectin expression. Am. J. Physiol. Heart Circ. Physiol. 279: H791-H797, 2000.

50. Gunnett, C. A., Heistad, D. D., Berg, D. J., and Faraci, F. M.: IL-10 deficiency increases superoxide and endothelial dysfunction during inflammation. Am. J. Physiol. Heart Circ. Physiol. 279: H1555-H1562, 2000.

51. Hille, R., and Nishino, T.: Xanthine oxidase and xanthine dehydrogenase. FASEB J. 9: 995-1003, 1995.

52. Nishino, T and Nishino, T.: The conversion from the dehydrogenase type to the oxidase type of rat liver xanthine dehydrogenase by modification of cysteine residues with fluorodinitrobenzene. J. Biol. Chem. 272: 29859-29864, 1997.

53. Enroth, C., Eger, B. T., Okamoto, K., Nishino, T., Nishino, T., and Pai, E. F.: Crystal structure of bovine milk xanthine dehydrogenase and xanthine oxidase: structure based mechanism of conversion. Proc. Natl. Acad. Sci. USA 97: 10723-10728, 2000.

54. Hille R, Massey V. Molybdenum containing hydroxylases: Xanthine oxidase, aldehyde oxidase, and sulfite oxidase. In Molybdenum Enzymes. T. G. Spiro, editor. John Wiley & Sons., New York, N. Y. 443-519, 1985.

55. Faure M, Lissi E A, Videla L A. Antioxidant capacity of allopurinol in biological systems. Biochem. Int. 1990; 21:356-366.

56. Zimmerman B J, Parks D A, Grisham M B, Granger D N. Allopurinol does not enhance antioxidant properties of extracellular fluid. Am. J. Physiol. 1988;255:H202-206.

57. Beswick, R. A., Dorrance, A. M., Leite, R., and Webb, R. C.: NADH/NADPH oxidase and enhanced superoxide production in the mineralcorticoid hypertensive rat. Hypertension 38: 1107-1111, 2001.

58. Li, L., Fink, G. D., Watts, S. W., Northcott, C. A., et al.: Endothelin-1 increases vascular superoxide via endothelin-NADPH oxidase pathway in low rennin hypertension. Circulation 107: 1053-1058, 2003.

59. Ungvari, Z., Csiszar, A., Edwards, J. G., Kaminski, P. M., et al.: Increased superoxide production in coronary arteries in hyperhomocysteinemia. Arterioscier. Thromb. Vasc. Biol. 23: 418-424, 2003.

60. Hille R. Molybdenum and tungsten in biology. Trends in Biochem. Sci. 27: 360-367, 2002.

61. Millar, T. M., Stevens, C. R., Benjamin, N., Eisenthal, R., et al.: Xanthine oxidoreductase catalyses the reduction of nitrates and nitrite to nitric oxide under hypoxic conditions. FEBS Lett. 427: 225-228, 1998.

62. Doel, J. J., Godber, B. L. J., Goult, T. A., Eisenthal, R., and Harrison, R.: Reduction of organic nitrites to nitric oxide catalyzed by xanthine oxidase: possible role in metabolism of nitrovasodilators. Biochem. Biophys. Res. Comm. 270: 880-885, 2000.

63. Godber, B. L. J., Doel, J. J., Sapkota, G. P., Blake, D. R., et al.: Reduction of nitrite to nitric oxide catalyzed by xanthine oxidoreductase. J. Biol. Chem. 275: 7757-7763, 2000.

64. Godber, B. L. J., Doel, J. J., Durgan, J., Eisenthal, R., and Harrison, R.: A new route to peroxynitrite: a role for xanthine oxidoreductase. FEBS Lett. 475: 93-96, 2000.

65. Li, H., Samouilov, A., Liu, X., and Zweier, J. L.: Characterization of the magnitude and kinetics of xanthine oxidase catalyzed nitrate reduction: evaluation of its role in nitrite and nitric oxide generation in anoxic tissues. Biochem. 42: 1150-1159, 2003.

66. McCord, J. M.: Oxygen derived free radicals in post-ischemic tissue injury. New Engl. J. Med. 312, 159-163, 1985.

67. Harrison, R.: Structure and finction of xanthine oxidoreductase: where are we now? Free Rad. Biol. Med. 33: 774-797, 2002.

68. Grosso, M. A., Brown, J. M., Viders, D. E., Mulvin, D. W., Banerjee, A., Velasco, S. E., Repine, J. E., and Harken, A. H.: Xanthine oxidase derived oxygen radicals induce pulmonary edema via direct endothelial injury. J. Surgical Res. 46: 355-360, 1989.

69. Terada, L. S., Willingham, I. R., Rosandich, M., Leff, J., Kindt, G., and Repine, J. E.: Generation of superoxide anion by brain endothelial xanthine oxidase. J. Cell. Physiol. 148:191-196, 1991.

70. Zweier, J. L., Broderick, R., Kuppusamy, P., Thompson-Gorman, S., and Lutty, G. A.: Determination of the mechanism of free radical generation in human aortic endothelial cells exposed to anoxia and reoxygenation. J. Biol. Chem. 269: 24156-24162, 1994.

71. Kayyali, U. S., Donaldson, C., Huang, H., Abdelnour, R., and Hassoun, P. M.: Phosphorylation of xanthine dehydrogenase/oxidase in hypoxia. J. Biol. Chem. 276: 14359-14365, 2001.

72. Bulkley, G. B.: Endothelial xanthine oxidase: a radical transducer of inflammatory signals for reticuloendothelial activation. Br. J. Surg. 80: 684-686, 1993.

73. Rieger, J. M., Shah, A. R., and Gidday, J. M.: Ischemia/reperfusion injury of retinal endothelium by cyclooxygenase and xanthine oxidase derived superoxide. Exp. Eye Res. 74: 493-501, 2002.

74. Kayyali, U. S., Budhiraja, R., Pennella, C. M., Cooray, S., et al.: Up regulation of xanthine oxidase by tobacco smoke condensate in pulmonary endothelial cells. Toxicol. App. Pharn. 188: 59-68, 2003.

75. Paler-Martinez, A., Panus, P. C., Chumley, P. H., Ryan, U., Hardy, M. M., and Freeman, B. A.: Endogenous xanthine oxidase does not significantly contribute to vascular endothelail production of reactive oxygen species. Arch. Biochem. Biophys. 311: 79-85, 1994.

76. Zulueta, J. J., Yu, F. S., Hertig, I. A., Thannickal, V. J., and Hassoun, P. M.: Release of hydrogen peroxide in response to hypoxia reoxygenation: role of NAD(P)H oxidase like enzyme in endothelial cell plasma membrane. Am. J. Respir. Cell Mol. Biol. 12: 41-49, 1995.

77. Parinandi, N. L., Kleinberg, M. A., Usatyuk, P. V., et al.: Hyperoxia induced NAD(P)H oxidase activation and regualtion by MAP kinases in human lung endothelial cells. Am. J. Physiol. Lung Cell Mol. Physiol. 284: L26-L38, 2002.

78. Pfeffer, K. D., Heucksteadt, T. P., and Hoidal, J. R.: Xanthine dehydrogenase and xanthine oxidase activity and gene expression in renal epithelial cells. J. Immuno. 153:1789-1797, 1994.

79. Page, S., Powell, D., Benboubetra, M., Stevens, C. R., Blake, D. R., Selase, F., Wolstenhoim, A. J., and Harrison, R.: Xanthine oxidoreductase in human mammary epithelial cells: activation in response to inflammatory cytokines. Biochem. Biophys. Acta 1381: 191-202, 1998.

80. Hassoun, P. M., Yu, F. S., Cote, C. G., Zulueta, J. J., Sawhney, R., Skinner, K. A., Skinner, H. B., Parks, D. A., and Lanzillo, J. J.: Upregulation of xanthine oxidase by lippopolysaccharide, interleukin-1, and hypoxia. Am. J. Respir. Crit. Care Med. 158: 299-305, 1998.

81. McManaman, J. L., Neville, M. C., and Wright, R. M.: Mouse mammary gland xanthine oxidoreductase: purification, characterization, and regulation. Arch. Biochem. Biophys. 371: 308-316, 1999.

82. Moriwaki, Y., Yamamoto, T., Suda, M., Masako, Y., Takahashi, S., Agbedana, O. E., Hada, T., and Hagashino, K.: Purification and immunohistochemical tissue localization of human xanthine oxidase. Biochem. Biophys. Acta 164:327-330, 1993.

83. Vorbach, C., Harrison, R., and Capecchi, M. R.: Xanthine oxidoreductase is central to the evolution and function of the innate immune system. Trends in Immunol. 24: 512-517, 2003.

84. Rosen, G. M., Pou, S., Ramos, C. L., Cohen, M. S., and Britigan, B. E.: Free Radicals and phagocytic cells. FASEB J. 9: 200-209, 1995.

85. Takao S, Smith, E. H., Wang, D., Chan, C. K., Bulkley, G. B., and Klein, A. S.: Role of reactive oxygen metabolites in murine peritoneal macrophage phagocytosis and phagocytic killing. Am. J. Physiol. 271: C1278-C1284, 1996.

86. Umezawa, K., Akaike, T., Fujii, S., Suga, M., et al.: Induction of nitric oxide synthsis and xanthine oxidase and their roles in the antimicrobial mechanism against Salmonella typhimurium infection in mice. Infect. hnmun. 65: 2932-2940, 1997.

87. Tubaro, E., Lotti, B., Santiangeli, C., and Cavallo, G.: Xanthine oxidase increases in polymorphonuclear leukocytes and macrophages in mice in three pathological situations. Biochem. Pharm. 29:1945-1948, 1980.

88. Segal, B. H., Sakamoto, N., Patel, M., Maemura, K., et al.: Xanthine oxidase contributes to host defense against Burkholderia cepacia in the p47^{phox4-/-}mouse model of chronic granulomatous disease. Infect. Immun. 68: 2374-2378, 2000.

89. Chow, C. W., Clark, M., Rinaldo, J., Chalkley, R.: Identification of the rat xanthine dehydrogenase /oxidase promoter. Nuc. Acids Res. 22:1846-1854, 1994.

90. Clark, M. P., Chow, C. W., Rinaldo, J. E., and Chalkley, R.: Multiple domains for initiator binding proteins TFII-I and YY-1 are present in the initiator and upstream regions of the rat XOR TATA-less promoter. Nuc. Acids Res. 26: 2813-2820, 1998.

91. Clark, M. P., Chow, C. W., Rinaldo, J. E., and Chalkley, R.: Correct usage of multiple transcription initiation sites and C/EBP dependent transcription activation of the rat XDH/XO TATA-less promoter requires downstream elements located in the coding region of the gene. Nuc. Acids Res. 26: 1801-1806, 1998.

92. Porcu, P., Cripe, L. D., Ng, E. W., Bhatia, S., et al.: Hyperleukocytic leukemias and leukostasis: a review of pathophysiology, clinical presentation, and management. Leuk. Lymphoma 39: 1-18, 2000.

93. Lascari, A. D.: Improvement of leukemic hyperleukocytosis with only fluid and allopurinol therapy. Am. J. Dis. Child. 145: 969-970, 1991.

94. Maurer, H. S., Steinherz, P. G., Gaynon, P. S., Finklestein, J. Z., et al.: The effect of initial management of hyperleukocytosis on early complications and outcome of children with acute lymphoblastic leukemia. J. Clin. Oncol. 6: 1425-1432, 1988.

95. Nelson, S. C., Bruggers, C. S., Kurtzberg, J., and Friedman, H. S.: Management of hyperleukocytosis with hydration, urinary alkalinization, and allopurinol. Are cranial irradiation and invasive cytoreduction necessary? Am. J. Pediatr. Hematol. Oncol. 15: 351-355, 1993.

96. Basade, M., Dhar, A. K., Kulkarni, S. S., Sastry, P. S., et al.: Rapid cytoreduction in childhood leukemic hyperleukocytosis by conservative therapy. Med. Pediatr. Oncol. 25: 204-207, 1995.

97. Masson, E., Synold, T. W., Relling, M. V., Schuetz, J. D., et al.: Allopurinol inhibits de novo purine synthesis in lymphoblasts of children with acute lymphoblastic leukemia. Leukemia 10: 56-60, 1996.

98. Beutler, B., and Rietschel, E. T.: Innate immune sensing and its roots: the story of endotoxin. Nat. Rev. Immuno. 3: 169-176, 2003.

99. Dobrovolskaia, M. A., and Vogel, S. N.: Toll receptors, CD14, and macrophage activation and deactivation by LPS. Microbes Infect. 4: 903-914, 2002.

100. Ehrt, S., Schnappinger, D., Bekiranov, S., et al.: Reprogramming the macrophage transcriptome in response to Interferon-y and Mycobacterium tuberculosis. J. Exp. Med. 194: 1123-1139, 2001.

101. Kovarik, P., Stoiber, D., Novy, M., and Decker, T.: STATI combines signals derived from IFN-y and LPS receptors during macrophage activation. The EMBO J. 17: 3660-3668, 1998.

102. Bach, E. A., Aguet, M., and Schreiber, R. D.: The IFN-y receptor: a paradigm for cytokine receptor signaling. Ann. Rev. Immunol. 15: 563-591, 1997.

103. Morimoto, K., Amano, H., Sonoda, F., et al.: Alveolar macrophages that phagocytose apoptotic neutrophils produce HGF during bacterial pneumonia in mice. Am. J. Respir. Cell Mol. Biol. 24: 608-615, 2001.

104. Gunn, M. D., Nelken, N. A., Liao, X., and Williams, L. T.: Monocyte chemoattractant protein-1 is sufficient for the chemotaxis of monocytes and lymphocytes in transgenic mice but requires an additional stimulus for inflammatory activation. J. Immunol. 158: 376-383, 1997.

105. Maus, U., Huwe, J., Maus, R., Seeger, W., and Lohmeyer, J.: Alveolar JE/MCP-1 and endotoxin synergize to provoke lung cytokine upregulation, sequential neutrophils and monocytes influx, and vascular leakage in mice. Am. J. Respir. Crit. Care Med. 164: 406-411, 2001.

106. Maus, U., vonGrote, K., Kuziel, W. A., Mack, M., et al.: The role of CC chemokine receptor 2 in alveolar monocytes and neutrophils immigration in intact mice. Am. J. Respir. Crit. Care Med. 166: 268-273, 2002.

107. Rosseau, S., Hammerl, P., Maus, U., et al.: Phenotypic characterization of alveolar monocyte recruitment in acute respiratory distress syndrome. Am. J. Physiol. Lung Cell Mol. Physiol. 279: L25-L35, 2000.

108. Rosseau, S., Selhorst, J., Wiechmann, K., et al.: Monocyte migration through the alveolar epithelial barrier: adhesion molecule mechanisms and impact of chemokines. J. Immuno. 164: 427-435, 2000.

109. Maus, U., Herold, S., Muth, H., et al.: Monocytes recruited into the alveolar air space of mice show a monocytic phenotype but upregulate CD 14. Am. J. Physiol. Lung Cell Mol. Physiol. 280: L58-L68, 2001.

110. Li, X. C., Miyasaka, M., and Issekutz, T. B.: Blood monocyte migration to acute lung inflammation involves both CD11/CD18 and very late activation antigen-4 dependent and independent pathways. J. Immuno. 161: 6258-6264, 1998.

111. Friedman, A. D.: Transcriptional regulation of granulocyte and monocyte development. Oncogene 21: 3377-3390, 2002.

112. Valledor, A. F., Borras, F. E., Cullell-Young, M., and Celeda, A.: Transcription factors that regulate monocyte/macrophage differentiation. J. Leukocyte Biol. 63: 405-417, 1998.

113. Coccia, E. M., Russo, N. D., Stellacci, E., Testa, U., Marziali, G., and Battistini, A.: STAT I activation during monocyte to macrophage maturation: role of adhesion molecules. Intemat. Immuno. 11: 1075-1083, 1999.

114. Skalnik, D. G.: Transcriptional mechanisms regulating myeloid specific genes. Gene 284: 1-21, 2002.

115. Detmer, K., and Walker, A. N.: Bone morphogenetic proteins act synergistically with hematopoietic cytokines in the differentiation of hematopoietic progenitors. Cytokine 17: 36-42, 2002.

116. Rosendahl, A., Pardali, E., Speletas, M., Dijke, P. T., Heldin, C. H., and Sideras, P.: Activation of bone morphogenetic protein/Smad signaling in bronchial epithelial cells during airway inflammation. Am. J. Respir. Cell Mol. Biol. 27: 160-169, 2002.

117. Tjin, R. M., Sjin, T., Krishnaraju, K., Hoffman, B., and Liebermann, D. A.: Transcriptional regulation of myeloid differentiation primary response (MYD) genes during myeloid differentiation is mediated by nuclear factor Y (NF-Y). Blood 100: 80-88, 2002.

118. Wetzel, M. A., Steele, A. D., Eisenstein, T. K., Adler, M. W., Henderson, E. E., and Rogers, T. J.: Mu-opioid induction of MCP-1, RANTES, and IFN-γ inducible protein 10 expression in human peripheral blood mononuclear cells. J. Immunol. 165: 6519-6524, 2000.

119. Gavrilin, M. A., Deucher, M. F., Boeckman, F., and Kolattukudy, P. E.: MCP-1 upregulates IL-1 expression in human monocytes. Biochem. Biophys. Res. Comm. 277: 37-42, 2000.

120. Foey, A. D., Feldman, M., and Brennan, F. M.: Route of monocyte differentiation determines their cytokine production profile. Cytokine 12: 1496-1505, 2000.

121. Ragno, S., Romano, M., Howell, S., Pappin, D. J., Jenner, P. J., and Colston, M. J.: Changes in gene expression in macrophages infected with Mycobacterium. Immunology 104, 99-108, 2001.

122. Carvalho-Pinto, C. E., Garcia, M. I., Mellado, M., et al.: Autocrine production of IFN-γ by macrophages controls their recruitment to kidney and the development of glomerulonephritis in MRlpr mice. J. Immunol. 169: 1058-1067, 2002.

123. Lucas, D. M., Lokuta, M. A., McDowell, A., Doan, J. E., and Paltnock, D. M.: Analysis of the IFN-γ signaling pathway in macrophages at different stages of maturation. J. Immunol. 160: 43374342, 1998.

124. Maus, U., Henning, H., Wenschuh, K., Mayer, W., et al.: Role of endothelial MCP-1 in monocytes adhesion to inflamed human endothelium under physiological flow. Am. J. Physiol. Heart Circ. Physiol. 283: H2584-H2591, 2002.

125. Robson, R. L., McLoughlin, R. M., Witowski, J., Loetscher, P., et al.: Differential regulation of chemokine production in human peritoneal mesothelial cells: IFN controls neutrophil migration across the mesothelium in vitro and in vivo. J. Immunol. 167:1028-1038, 2001.

126. Maus, U., Koay, M. A., Delbeck, T., Mack, M., et al.: Role of resident alveolar macrophages in leukocyte traffic into the alveolar air space of intact mice. Am. J. Physiol. Lung Cell Mol. Physiol. 282: L1245-L1252, 2002.

127. Maus, U., Waelsch, K., Kuziel, W. A., Delbeck, T., et al.: Monocytes are potent facilitators of alveolar neutrophil migration during lung inflammation. Role of the CCL2-CCR2 axis. J. Immunol. 170: 3273-3278, 2003.

128. Varney, M. L., Olsen, K. J., Mosley, R. L., et al.: Monocyte/macrophage recruitment, activation and differentiation modulate IL-8 production. In vivo 16: 471-477, 2002.

129. Hunninghake, G. W., Gallin, J. I., and Fauci, A. S.: Immunologic reactivity of the lung: the in vivo and in vitro generation of a neutrophil chemotactic factor by alveolar macrophages. Am. Rev. Respir. Dis. 117: 15-23, 1978.

130. Hunninghake, G. W., Gadek, J. E., Lawley, T. J., and Crystal, R. G.: Mechanism of neutrophil accumulation in the lungs of patients with idiopathic pulmonary fibrosis. J. Clin. Invest. 68: 259-262, 1981.

131. Chisolm, G. M., Hazen, S. L., Fox, P. L., and Cathcart, M. K.: The oxidation of lipoproteins by monocytes-macrophages. J. Biol. Chem. 274: 25959-25962, 1999.

132. Bey, E. A. and Cathcart, M. K.: In vitro knockout of human p47phox blocks superoxide anion production and LDL oxidation by activated human monocytes. J. Lipid Res. 41: 489-495, 2000.

133. Cathcart, M. K.: Regulation of superoxide anion production by NADPH oxidase in monocytes/macrophages. Contribution to atherosclerosis. Arterioscler. Thromb. Vasc. Biol. 23: 1-6, 2003.

134. Forman, H. J. and Torres, M.: Redox signaling in macrophages. Mol. Aspects Med. 22: 189-216, 2001.

135. Forman, H. J. and Torres, M.: Reactive oxygen species and cell signaling. Respiratory burst in macrophage signaling. Am. J. Respir. Crit. Care Med. 166: S4-S8, 2002.

136. Arnold, R. S., Shi, J., Murad, E., Whalen, A. M., et al.: Hydrogen peroxide mediates the cell growth and transformation caused by the mitogenic oxidase Noxl. PNAS 98: 5550-5555, 2001.

137. Behrend, L., Henderson, G., and Zwacka, R. M.: Reactive oxygen species in oncogenic transformation. Biochem. Soc. Trans. 31:1441-1444, 2003.

138. Zhu, J., and Emerson, S. G.: Hematopoietic cytokines, transcription factors and lineage commitment. Oncogene 21: 3295-3313, 2002.

139. Martinez-Moczygemba, M. and Huston, D. P.: Biology of common β-receptor signaling cytokines: IL-3, IL-5, and GM-CSF. J. Allergy Clin. Immunol. 112: 653-665, 2003.

140. Trapnell, B. C. and whitsett, J. A.: GM-CSF regulates pulmonary surfactant homeostasis and alveolar macrophage mediated innate host defense. Ann. Rev. Physiol. 64: 775-802, 2002.

141. Sattler, M., Winkler, T., Verma, S., Byrne, C. H., et al.: Hematopoietic growth factors signal through the formation of reactive oxygen species. Blood 93: 2928-2935, 1999.

142. Carcamo, J. M., Ojeda, O. B., and Golde, D. W.: Vitamin C inhibits GM-CSF induced signaling pathways. Blood 99: 3205-3212, 2002.

143. Bozinovski, S., Jones, J., Beavitt, S. J., Cook, A. D., et al.: Innate immune responses to LPS in mouse lung are suppressed and reversed by neutralization of GM-CSF via repression of TLR4. Am. J. Physiol. Lung Cell Mol. Physiol. E-pub. 00275, 2003.

144. Bitterman, P. B., Saltzman, L. E., Adelberg, S., Ferrans, V. J., and Crystal, R. G.: Alveolar macrophage replication. One mechanism for the expansion of the mononuclear phagocyte population in the chronically inflamed lung. J. Clin. Invest. 74: 460-469, 1984.

145. Martin, T. R.: Lung cytokines and ARDS. Chest 116: 2-8, 1999.

146. Shimabukuro, D. W., Sawa, T., and Gropper, M. A.: Injury and repair in lung and airways. Crit. Care Med. 31: S524-S531, 2003.

147. Goodman, R. B., Pugin, J., Lee, J. S., and Matthay, M. A.: Cytokine mediated inflammation in acute lung injury. Cytokine Growth Fact. Rev. 14: 523-535, 2003.

148. Krakauer, T.: Pentoxifylline inhibits ICAM-1 expression and chemokine production induced by proinflammatory cytokines in human pulmonary epithelial cells. Immunopharm. 46: 253-261, 2000.

149. Leff, J. A., Baer, J. W., Bodman, M. E., Kirkman, J. M., et al.: Interleukin-1 induced lung neutrophil accumulation and oxygen metabolite mediated lung leak in rats. Am. J. Physiol. (Lung Cell. Mol. Physiol. ) 266: 2-8, 1994.

150. Guidot, D. M., Stevens, E. E., Repine, M. J., Lucca-Broco, A. E. and Repine, J. E.: Intratracheal but not intravascular interleukin-1 causes acute edematous injury in isolated neutrophil-perfused rat lungs through an oxygen radical mediated mechanism. J. Lab. Clin. Med. 123:605-609, 1994.

151. Zhang, P., Quniton, L. J., Bagby, G. J., Summer, W. R., and Nelson, S.: Interferon-y enhances the pulmonary CXC chemokine response to intratracheal lipopolysaccharide challenge. J. Infect. Dis. 187: 62-69, 2003.

152. Ribeiro, R. A., Cunha, F. Q., and Ferreira, S. H.: Recombinant gamma interferon causes neutrophil migration mediated by the release of a macrophage neutrophil chemotactic factor. Int. J. Exp. Path. 71: 717-725, 1990.

153. Wen, L. P., Madani, K., Fahrni, J. A., Duncan, S. R., and Rosen, G. D.: Dexamethasone inhibits lung epithelial cell apoptosis induced by IFN-γ and Fas. Amer. J. Physiol: Lung Cell. Molec. Physiol. 273: L921-L929, 1997.

154. Terao, M., Cazzaniga, G., Ghezzi, P., Bianchi, M., Falciani, F., Perani, P., and Garattini, E.: Molecular cloning of a cDNA coding for mouse liver xanthine dehydrogenase. Regulation of its transcript by interferons in vivo. Biochem. J. 283: 863-870, 1992.

155. Falciani, F., Ghezzi, P., Terao, M., Cazzaniga, G., and Garattini, E.: Interferons induce xanthine dehydrogenase gene expression in L929 cells. Biochem. J. 285: 1001-1008, 1992.

156. McManaman, J. L., Hanson, L., Neville, M. E., and Wright, R. M.: Lactogenic hormones regulate xanthine oxidoreductase and beta casein levels in mammary epithelial cells by distinct mechanisms. Arch. Biochem. Biophys. 373: 318-327, 2000.

157. Kurosaki, M., Zanotta, S., Calzi, M. C., Garattini, E., and Terao, M.: Expression of xanthine oxidoreductase in mouse mammary epithelium during pregnancy and lactation. Biochem. J. 319: 801-810, 1996.

158. Terada, L. S., Piermattei, D., Shibao, G. N., McManaman, J. L., and Wright, R. M.: Hypoxia regulates xanthine dehydrogenase activity at pre- and post-translational levels. Arch. Biochem. Biophys. 348:163-168, 1997.

159. Xu, P., LaVallee, P., and Hoidal, J. R.: Repressed expression of the human xanthine oxidoreductase gene. J. Biol. Chem. 275: 5918-5926, 2000.

160. Martelin, E., Palvimo, J. J., Lapatto, R., and Raivio, K. O.: Nuclear factor Y activates the human xanthine oxidoreductase gene promoter. FEBS Lett. 480: 84-88, 2000.

161. Newman, L. S., Rose, C. S. and Maier, L. A.: Sarcoidosis. New England J Med 336: 1224-1234, 1997.

162. Thomas, K. W. and Hunninghake, G. W.: Sarcoidosis. J Am Med Assoc 289: 3300-3303, 2003.

163. Agostini, C, Facco, M., Chilosi, M., Semenzato, G.: Alveolar macrophage-T cell interactions during Th-1 type sarcoid inflammation. Microsc Res Tech 53: 278-287, 2001.

164. Prince, J. E., Kheradmand, F., and Corry, D. B.: Immunologic lung disease. J Allergy Clin Immunol 111: 613-623, 2003.

165. Kunkel, S. L., Lukacs, N. W., Strieter, R. M., and Chensue, S. W.: Th1 and Th2 responses regulate experimental lung granuloma development. Sarcoidosis Vasc Diffuse Lung Dis 13: 120-128, 1996.

166. Wahlstrom, J., Berlin, M., Skold, C. M., Wigzell, H., Eklund, A., and Grunewald, J.: Phenotypic analysis of lymphocytes and monocytes/macrophages in peripheral blood and bronchoalveolar lavage fluid from patients with pulmonary sarcoidsis. Thorax. 54: 339-346, 1999.

167. Thole, A. A., Rodrigues, C. A., Milward, G., Negreiros, Porto, L. C., and Carvalho, L.: Ultrastructural study of expression of adhesion molecules between blood monocytes and alveolar macrophages from patients with pulmonary sarcoidosis. J Submicrosc Cytol Pathol. 33: 419-424, 2001.

168. Dalhoff, K., Bohnet, S., Braun, J., Kreft, B., and Wiessmann, K. J.: Intercellular adhesion molecule 1 (ICAM-1) in the pathogenesis of mononuclear cell alveolitis in puhnonary sarcoidosis. Thorax. 48: 1140-1144, 1993.

169. Lenz, A. G., Costabel, U., and Maier, K. L.: Oxidized BAL fluid proteins in patients with interstitial lung diseases. Eur Resp J 9: 307-312, 1996.

170. Moodley, Y. P., Chetty, R., and Lalloo, U. G.: Nitric oxide levels in exhaled air and inducible nitric oxide synthase immunolocalization in pulmonary sarcoidosis. Eur Resp J 14: 822-827, 1999.

171. Lakari, E., Paakko, P., Pietarinen-Runtti, P., and Kinnula, V. L.: Manganese superoxide dismutase and catalase are coordinately expressed in the alveolar region in chronic interstitial pneumonia and granulomatous diseases of the lung. Am J Respir Crit Care Med 161: 615-621, 2000.

172. Aerts, C., Wallaert, B., Grosbois, J. M., and Voisin, C.: Release of superoxide anion by alveolar macrophages in pulmonary sarcoidosis. Ann NY Acad Sci 465: 193-200, 1986.

173. Barth, J., Entzian, P., and Petermann, W.: Increased release of free oxygen radicals by phagocytosing and nonphagocytosing cells from patients with active pulmonary sarcoidosis as revealed by luminol-dependent chemiluminescence. Klin Wochenschr. 666: 292-297, 1988.

174. Calhoun, W. J., Salisbury, S. M., Chosy, L. W., and Busse, W. W.: Increased alveolar macrophage chemiluminescence and airspace superoxide production in active pulmonary sarcoidosis. J Lab Clin Med. 112: 147-156, 1988.

175. Cassatella, M. A, Berton, G., Agostini, C., Zambello, R., Trentin, L., Cipriani, A., and Semenzato, G.: Generation of superoxide anion by alveolar macrophages in sarcoidosis: evidence for the activation of the oxygen metabolism in patients with high intensity alveolitis. Immunol. 666: 451-458, 1989.

176. Schaberg, T., Rau, M., Stephan, H., and Lode, H.: Increased numbers of alveolar macrophages expressing surface molecules of the CD/11CD18 family in sarcoidosis and idiopathic pulmonary fibrosis is related to the production of superoxide anions by these cells. AM Rev Respir Dis 147: 1507-1513, 1993.

177. Rhaman, I.: Oxidative stress, chromatin remodeling, and gene transcription in inflammation and chronic lung disease. J Biochem Mol Biol 36: 95-109, 2003.

178. MacNee, W.: Oxidative stress and lung inflammation in airways disease. Eur J Pharm 429:195-207, 2001.

179. Pinamonti, S., Muzzoli, M., Chicca, M. C., Papi, A., Ravenna, F., Fabbri, L. M., and Ciaccia, A.: Xanthine oxidase activity in bronchoalveolar lavage fluid from patients with chronic obstructive pulmonary disease. Free Rad Biol Med 21: 147-155, 1996.

180. Pinamonti, S., Leis, M., Barbieri, A., Leoni, D., Muzzoli, M., Sostero, S., Chicca, M. C., Carrieri, A., Ravenna, F., Fabbri, L. M., and Ciaccia, A.: Detection of xanthine oxidase activity products by EPR and HPLC in bronchoalveolar lavage fluid from patients with chronic obstructive pulmonary disease. Free Rad Biol Med 25: 771-779, 1998.

181. Heunks, L. M., Vina, J., Herwaarden, C. L. ., Folgering, H. T., Gimeno, A., and Dekhuijzen, P. P. N. .: Xanthine oxidase is involved in exercise induced oxidative stress in chronic obstructive pulmonary disease. Am J Phyisol 277:R1697-1704, 1999.

182. Pfau, A., Stolz, W., Karrer, S., Szeimies, R. M., and Landthaler, M.: Allopurinol treatment of cutaneous sarcoidosis. Hautarzt 49: 216-218, 1998.

183. Okamoto, H., Mizuno, K., and Horio, T.: Monocyte derived multinucleated giant cells and sarcoidosis. J Dermatol Sci 31: 119-128, 2003.

184. Mizuno, K., Okamoto, H., and Horio, T.: Inhibitory influences of xanthine oxidase inhibitor and ACE inhibitor on multinucleate giant cell formation from monocytes by down regulation of adhesion molecules and purinergic receptors. Br J Dermatol (In press).

185. Antony, F., and Layton, A. M.: A case of cutaneous acral sarcoidosis with response to allopurinol. Br J Dermatol. 142: 1052-1053, 2000.

186. El-Euch, D., Mokni, M., Trojjet, S., Khouaja, A., and Ben-Osman, A.: Sarcoidosis in a child treated successfully with allopurinol. Br J Dermatol 140: 1184-1185, 1999.

187. Hille, R., and Nishino, T.: Xanthine oxidase and xanthine dehydrogenase. FASEB J 9: 995-1003, 1995.

188. Nishino, T and Nishino, T.: The conversion from the dehydrogenase type to the oxidase type of rat liver xanthine dehydrogenase by modification of cysteine residues with fluorodinitrobenzene. J. Biol. Chem. 272: 29859-29864, 1997.

189. Enroth, C., Eger, B. T., Okamoto, K., Nishino, T., Nishino, T., and Pai, E. F.: Crystal structure of bovine milk xanthine dehydrogenase and xanthine oxidase: structure based mechanism of conversion. Proc. Natl. Acad. Sci. USA 97: 10723-10728, 2000.

190. Quinlan, G. J., Lamb, N. J., Tilley, R., Evans, T. W., and Gutteridge, J. M. C.: Plasma hypoxanthine levels in ARDS: implications for oxidative stress, morbidity, and mortality. Am. J. Resp. Crit. Care Med. 155: 479-484, 1997.

191. Grum, C. M., R. A. Ragsdale, L. H. Ketai, and R. H. Simon.: Plasma xanthine oxidase activity in patients with adult respiratory distress syndrome. J. Crit. Care 2:22-27, 1987.

192. Chinnaiyan, A. M., Huber-Lang, M., Kumar-Sinha, C., Barrette, T. R. et al.: Molecular signatures of sepsis. Multiorgan gene expression profiles of systemic inflammation. Am. J. Path. 159: 1199-1209, 2001.

193. Anderson, B. O., Moore, E. E., Moore, F. A., Leff, J. A., Terada, L. S., Harken, A. H., and Repine, J. E.: Hypovolemic shock promotes neutrophil sequestration in lungs by a xanthine oxidase related mechanism. J. Appl. Physiol. 71: 1862-1865, 1991.

194. Shenkar, R. and Abraham, E.: Plasma from hemorrhaged mice activates CREB and increases cytokine expression in lung mononuclear cells through a xanthine oxidase-dependent mechanism. Am. J. Respir. Cell Mol. Biol. 14:198-206, 1996.

195. Modelska, K., Matthay, M. A., Brown, L. A. S., Deutch, E., Lu, L. N., and Pittet, J. F.: Inhibition of B-adrenergic-dependent alveolar epithelial clearance by oxidant mechanisms after hemorrhagic shock. Am. J. Physiol. 276:L844-L857, 1999.

196. Adkins, W. K. and Taylor, A. E.: Role of xanthine oxidase and neutrophils in ischemia reperfusion injury in rabbit lung. J. Appl. Physiol. 69: 2012-2018, 1990.

197. Shibata, K., Cregg, N., Engelberts, D., Takeuchi, A., Fedorko, L., and Kavanagh, B. P.: Hypercapnic acidosis may attenuate acute lung injury by inhibition of endogenous xanthine oxidase. Am. J. Resp. Crit. Care Med. 158: 1578-1584, 1998.

198. Desco, M. C., et al.: Xanthine oxidase is involved in free radical production in type 1 diabetes: protection by allopurinol. Diabetes 51:1118-1124, 2002.

199. Butler, R., et al.: Allopurinol normalizes endothelial dysftmction in type 2 diabetes with mild hypertension. Hypertension 35:746-751, 2000.

200. Rhoden, E., et al.: Protective effect of allopurinol in the renal ischemia-reperfusion in uninephrectomized rats. Gen. Pharm 35:189-193, 2002.

201. Cappola, T. P., et al.: Allopurinol improves myocardial efficiency in patients with idopathic dilated cardiomyopathy. Circulation 104:2407-2411, 2001.

202. Doehner, W., et al.: Effects of xanthine oxidase inhibition with allopurinol on endothelial finction and peripheral blood flow in hyperuricemic patients with chronic heart failure. Circulation 105:2619-2624, 2002.

203. Farquharson, C. A. J., et al.: Allopurinol improves endothelial dysfimction in chronic heart failure. Circulation 106:221-226, 2002.

204. McCord, J. M.: Oxygen derived free radicals in post-ischemic tissue injury. New Engl. J. Med. 312, 159-163, 1985.

205. Harrison, R.: Structure and finction of xanthine oxidoreductase: where are we now? Free Rad. Biol. Med. 33: 774-797, 2002.

206. Grosso, M. A., Brown, J. M., Viders, D. E., Mulvin, D. W., Banerjee, A., Velasco, S. E., Repine, J. E., and Harken, A. H.: Xanthine oxidase derived oxygen radicals induce pulmonary edema via direct endothelial injury. J. Surgical Res. 46: 355-360, 1989.

207. Terada, L. S., Willingham, I. R., Rosandich, M., Leff, J., Kindt, G., and Repine, J. E.: Generation of superoxide anion by brain endothelial xanthine oxidase. J. Cell. Physiol. 148: 191-196, 1991.

208. Zweier, J. L., Broderick, R., Kuppusamy, P., Thompson-Gorman, S., and Lutty, G. A.: Determination of the mechanism of free radical generation in human aortic endothelial cells exposed to anoxia and reoxygenation. J. Biol. Chem. 269: 24156-24162, 1994.

209. Kayyali, U. S., Donaldson, C., Huang, H., Abdelnour, R., and Hassoun, P. M.: Phosphorylation of xanthine dehydrogenase/oxidase in hypoxia. J. Biol. Chem. 276: 14359-14365, 2001.

210. Bulkley, G. B.: Endothelial xanthine oxidase: a radical transducer of inflammatory signals for reticuloendothelial activation. Br. J. Surg. 80: 684-686, 1993.

211. Paler-Martinez, A., Panus, P. C., Chumley, P. H., Ryan, U., Hardy, M. M., and Freeman, B. A.: Endogenous xanthine oxidase does not significantly contribute to vascular endothelail production of reactive oxygen species. Arch. Biochem. Biophys. 311: 79-85, 1994.

212. Zulueta, J. J., Yu, F. S., Hertig, I. A., Thannickal, V. J., and Hassoun, P. M.: Release of hydrogen peroxide in response to hypoxia reoxygenation: role of NAD(P)H oxidase like enzyme in endothelial cell plasma membrane. Am. J. Respir. Cell Mol. Biol. 12: 41-49, 1995

213. Parinandi, N. L., Kleinberg, M. A., Usatyuk, P. V., et al.: Hyperoxia induced NAD(P)H oxidase activation and regualtion by MAP kinases in human lung endothelial cells. Am. J. Physiol. Lung Cell Mol. Physiol. 284: L26-L38, 2002.

214. Pfeffer, K. D., Heucksteadt, T. P., and Hoidal, J. R.: Xanthine dehydrogenase and xanthine oxidase activity and gene expression in renal epithelial cells. J. Immuno. 153:1789-1797, 1994.

215. Page, S., Powell, D., Benboubetra, M., Stevens, C. R., Blake, D. R., Selase, F., Wolstenholm, A. J., and Harrison, R.: Xanthine oxidoreductase in human mammary epithelial cells: activation in response to inflammatory cytokines. Biochem. Biophys. Acta 1381: 191-202, 1998.

216. Hassoun, P. M., Yu, F. S., Cote, C. G., Zulueta, J. J., Sawhney, R., Skinner, K. A., Skinner, H. B., Parks, D. A., and Lanzillo, J. J.: Upregulation of xanthine oxidase by lippopolysaccharide, interleukin-1, and hypoxia. Am. J. Respir. Crit. Care Med. 158: 299-305, 1998.

217. McManaman, J. L., Neville, M. C., and Wright, R. M.: Mouse mammary gland xanthine oxidoreductase: purification, characterization, and regulation. Arch. Biochem. Biophys. 371: 308-316, 1999.

218. Moriwaki, Y., Yamamoto, T., Suda, M., Masako, Y., Takahashi, S., Agbedana, O. E., Hada, T., and Hagashino, K.: Purification and immunohistochemical tissue localization of human xanthine oxidase. Biochem. Biophys. Acta 164:327-330, 1993.

219. Pantelidis, P., McGrath, D. S., Southcott, A. M., and du-Bois, R. M.: Single cell analysis: a novel approach to tumor necrosis factor-alpha synthesis and secretion in sarcoidosis. Eur Respir J 20: 1179-1184, 2002.

220. Fehrenbach, H., Zissel, G., Goldman, T., Tschernig, T., Vollmer, E., Pabst, R., and Muller-Querheim, J.: Alveolar macrophages are the main source for tumor necrosis factor-alpha in patients with sarcoidosis. Eur Respir J 21: 421-428, 2003.

221. Mikuniya, T., Nagai, S., Takeuchi, M., Mio, T., Hoshino, Y., Miki, H., Shigematsu, M., Hamada, K., and Izumi, T.: Quantitative evaluation of the IL-1 beta and IL-1 receptor antafonist obtained from BALF macrophages in patients with interstitial lung diseases. Sarcoidosis Vasc Diffuse Lung Dis. 14: 39-45, 1997.

222. Mikuniya, T., Nagai, S., Takeuchi, M., Mio, T., Hoshino, Y., Miki, H., Shigematsu, M., Hamada, K., and Izumi, T.: Significance of the interleukin-1 receptor antagonist/interleukin-1 beta ratio as a prognostic factor in patients with pulmonary sarcoidosis. Respiration 67: 389-396, 2000.

223. Kolb, M., Margetts, P. J., Anthony, D. C., Pitossi, F. and Gauldie, J.: Transient expression of IL-1beta induces acute lung injury and chronic repair leading to pulmonary fibrosis. J Clin Invest 107: 1529-1536, 2001.

224. Capelli, A., DiStefano, A., Gnemmi, I., Balbo, P., Cerutti, C. G., Balbi, B., Lusuardi, M., and Donner, C. F.: Increased MCP-1 and MIP-1 beta in bronchoalveolar lavage fluid of chronic bronchitics. Eur Respir J 14: 160-165, 1999.

225. Petrek, M., Kolek, V., Szotkowska, J., and DuBois, R. M.: CC and C chemokine expression in pulmonary sarcoidosis. Eur Respir J 20: 1206-1212, 2002.

226. Capelli, A., DiStefanop, A., Lusuardi, M., Gnemmi, I., and Donner, C. F.: Increased MIP-1alpha and MIP-1beta levels in bronchoalveolar lavage fluid of patients affected by different stages of pulmonary sarcoidosis. Am J Respir Crit Care Med 165: 236-241, 2002.

227. Gibejova, A., Mrazek, F., Subrtova, D., Sekerova, V., Szotkowska, J., Kolek, V., DuBois, R. M., and Petrek, M.: Expression of macrophage inflammatory protein-3beta in pulmonary sarcoidosis. Am J Respir Crit Care Med 167: 1695-1703, 2003.

228. Roman, J., Jeon, Y. J., Gal, A., and Perez, R. L.: Distribution of extracellular matrices, matrix receptors, and TGF-beta1 in human and experimental lung granulomatous inflammation. Am J Med Sci 309: 124-133, 1995.

229. Leff, J. A., Bodman, M. E., Cho, O. J., Rohrbach, S., Reiss, O. K., Vannice, J. L and Repine, J. E.: Post-insult treatment with interleukin-1 receptor antagonist decreases oxidative lung injury in rats given intratracheal interleukin-1. Amer. J. Respir. Crit. CareMed. 150:109-112, 1994.

230. Krakauer, T.: Pentoxifylline inhibits ICAM-1 expression and chemokine production induced by proinflammatory cytokines in human pulmonary epithelial cells. Immunopharm. 46: 253-261, 2000.

231. Steinmuller, C., Franke-Ullman, G., Lohmann-Matthes, M. L., and Emmendorffer, A.: Local activation of nonspecific defense against a respiratory model infection by application of interferon-gamma. Am. J. Respir. Cell Mol. Biol. 22: 481-490, 2000.

232. Terao, M., Cazzaniga, G., Ghezzi, P., Bianchi, M., Falciani, F., Perani, P., and Garattini, E. Molecular cloning of a cDNA coding for mouse liver xanthine dehydrogenase. Regulation of its transcript by interferons in vivo. Biochem. J. 283: 863-870, 1992.

234. Falciani, F., Ghezzi, P., Terao, M., Cazzaniga, G., and Garattini, E.: Interferons induce xanthine dehydrogenase gene expression in L929 cells. Biochem. J. 285: 1001-1008, 1992.

235. McManaman, J. L., Hanson, L., Neville, M. E., and Wright, R. M.: Lactogenic hormones regulate xanthine oxidoreductase and beta casein levels in mammary epithelial cells by distinct mechanisms. Arch. Biochem. Biophys. 373: 318-327, 2000.

236. Terada, L. S., Piermattei, D., Shibao, G. N., McManaman, J. L., and Wright, R. M.: Hypoxia regulates xanthine dehydrogenase activity at pre- and post-translational levels. Arch. Biochem. Biophys. 348:163-168, 1997.

237. Zhao, M. Q., Stoler, M. H., Liu, A. N., Wie, B., Soguero, C., Hahn, Y. S., and Enelow, R. I.: Alveolar epithelial cell chemokine expression triggered by antigen specific cytolytic CD8+T cell recognition. J. Clin. Invest. 106: R49-R58, 2000.

238. Zhao, M. Q., Foley, M. P., Stoler, M. H., and Enelow, R. I.: Alveolar epithelial cell chemokine expression induced by specific antiviral CD8+T cell recognition plays a critical role in the perpetuation of experimental interstitial pneumonia. Chest 120:1 S-13S, 2001.

239. Ehrt, S., Schnappinger, D., Bekiranov, S., et al.: Reprogramming the macrophage transcriptome in response to Interferon-γ and Mycobacterium tuberculosis. J. Exp. Med. 194: 1123-1139, 2001.

240. Kovarik, P., Stoiber, D., Novy, M., and Decker, T.: STATI combines signals derived from IFN-γ and LPS receptors during macrophage activation. The EMBO J. 17: 3660-3668, 1998.

241. Bach, E. A., Aguet, M., and Schreiber, R. D.: The IFN-γ receptor: a paradigm for cytokine receptor signaling. Ann. Rev. Immunol. 15: 563-591, 1997.

242. Rosseau, S., Hammerl, P., Maus, U., et al.: Phenotypic characterization of alveolar monocyte recruitment in acute respiratory distress syndrome. Am. J. Physiol. Lung Cell Mol. Physiol. 279: L25-L35, 2000.

243. Rosseau, S., Selhorst, J., Wiechmann, K., et al.: Monocyte migration through the alveolar epithelial barrier: adhesion molecule mechanisms and impact of chemokines. J. Immuno. 164: 427-435, 2000.

244. Maus, U., Herold, S., Muth, H., et al.: Monocytes recruited into the alveolar air space of mice show a monocytic phenotype but upregulate CD14. Am. J. Physiol. Lung Cell Mol. Physiol. 280: L58-L68, 2001.

245. Li, X. C., Miyasaka, M., and Issekutz, T. B.: Blood monocyte migration to acute lung inflammation involves both CD11/CD18 and very late activation antigen-4 dependent and independent pathways. J. Immuno. 161: 6258-6264, 1998.

246. Friedman, A. D.: Transcriptional regulation of granulocyte and monocyte development. Oncogene 21: 3377-3390, 2002.

247. Valledor, A. F., Borras, F. E., Cullell-Young, M., and Celeda, A.: Transcription factors that regulate monocyte/macrophage differentiation. J. Leukocyte Biol. 63: 405-417, 1998.

248. Coccia, E. M., Russo, N. D., Stellacci, E., Testa, U., Marziali, G., and Battistini, A.: STAT1 activation during monocyte to macrophage maturation: role of adhesion molecules. Internat. Immuno. 11: 1075-1083, 1999.

249. Skalnik, D. G.: Transcriptional mechanisms regulating myeloid specific genes. Gene 284: 1-21, 2002.

250. Detmer, K., and Walker, A. N.: Bone morphogenetic proteins act synergistically with hematopoietic cytokines in the differentiation of hematopoietic progenitors. Cytokine 17: 36-42, 2002.

251. Rosendahl, A., Pardali, E., Speletas, M., Dijke, P. T., Heldin, C. H., and Sideras, P.: Activation of bone morphogenetic protein/Smad signaling in bronchial epithelial cells during airway inflammation. Am. J. Respir. Cell Mol. Biol. 27: 160-169, 2002.

252. Tjin, R. M., Sjin, T., Krishnaraju, K., Hoffinan, B., and Liebermann, D. A.: Transcriptional regulation of myeloid differentiation primary response (MYD) genes during myeloid differentiation is mediated by nuclear factor Y (NF-Y). Blood 100: 80-88, 2002.

253. Wetzel, M. A., Steele, A. D., Eisenstein, T. K., Adler, M. W., Henderson, E. E., and Rogers, T. J.: Mu-opioid induction of MCP-1, RANTES, and IFN-γ inducible protein 10 expression in human peripheral blood mononuclear cells. J. Immunol. 165: 6519-6524, 2000.

254. Gavrilin, M. A., Deucher, M. F., Boeckman, F., and Kolattukudy, P. E.: MCP-1 upregulates IL-1 expression in human monocytes. Biochem. Biophys. Res. Comm. 277: 37-42, 2000.

255. Foey, A. D., Feldman, M., and Brennan, F. M.: Route of monocyte differentiation determines their cytokine production profile. Cytokine 12: 1496-1505, 2000.

256. Ragno, S., Romano, M., Howell, S., Pappin, D. J., Jenner, P. J., and Colston, M. J.: Changes in gene expression in macrophages infected with Mycobacterium. Immunology 104, 99-108, 2001.

257. Carvalho-Pinto, C. E., Garcia, M. I., Mellado, M., et al.: Autocrine production of IFN-γ by macrophages controls their recruitment to kidney and the development of glomerulonephritis in MRL/1pr mice. J. Immunolo. 169: 1058-1067, 2002.

258. Lucas, D. M., Lokuta, M. A., McDowell, A., Doan, J. E., and Patlnock, D. M.: Analysis of the IFN-γ signaling pathway in macrophages at different stages of maturation. J. Immunol. 160: 4337-4342, 1998.

259. Varney, M. L., Olsen, K. J., Mosley, R. L., et al.: Monocyte/macrophage recruitment, activation and differentiation modulate IL-8 production. In Vivo 16: 471-477, 2002.

260. Chow, C. W., Clark, M., Rinaldo, J., Chalkley, R.: Identification of the rat xanthine dehydrogenase /oxidase promoter. Nuc. Acids Res. 22:1846-1854, 1994.

261. Clark, M. P., Chow, C. W., Rinaldo, J. E., and Chalkley, R.: Multiple domains for initiator binding proteins TFII-I and YY-1 are present in the initiator and upstream regions of the rat XOR TATA-less promoter. Nuc. Acids Res. 26: 2813-2820, 1998.

262. Clark, M. P., Chow, C. W., Rinaldo, J. E., and Chalkley, R.: Correct usage of multiple transcription initiation sites and C/EBP dependent transcription activation of the rat XDH/XO TATA-less promoter requires downstream elements located in the coding region of the gene. Nuc. Acids Res. 26: 1801-1806, 1998.

263. Martelin, E., Palvimo, J. J., Lapatto, R., and Raivio, K. O.: Nuclear factor Y activates the human xanthine oxidoreductase gene promoter. FEBS Lett. 480: 84-88, 2000.

264. Rinaldo, J. E., Clark, M., Parinello, J., and Shepherd, V. L.: Nitric oxide inactivates xanthine dehydrogenase and xanthine oxidase in interferon-y stimulated macrophages. Am. J. Respir. Cell Mol. Biol. 11: 625-630, 1994.

265. Takao S, Smith, E. H., Wang, D., Chan, C. K., Bulkley, G. B., and Klein, A. S.: Role of reactive oxygen metabolites in murine peritoneal macrophage phagocytosis and phagocytic killing. Am. J. Physiol. 271: C1278-C1284, 1996.

266. Wright, R. M., Ginger, L. A., Kosila, N., Elkins, N. E., Essary, B., McManaman, J. L., and Repine, J. E.: Mononuclear phagocyte xanthine oxidoreductase contributes to cytokine induced acute lung injury. Am J Respir Cell Mol Biol (In Press) 29: 2003.

267. Shirai, M., Sato, A., and Chida, K.: The influence of ovarian hormones on the granulomatous inflammatory process in the rat lung. Eur Respir J 8: 272-277, 1995.

268. Tsuchiya, T., Chida, K., Suda, T., Scheenberger, E. E., and Nakamura, H.: Dendritic cell involvement in pulmonary granuloma formation elicited by BCG in rats. Am J Respir Crit Care Med 165: 1640-1646, 2002.

269. Pfau, A., Abd-el-Raheem, T., and Landthaler, M.: Positive tuberculin reaction in sarcoidosis. Hautarzt 46: 250-254, 1995.

270. Moore, K. W., Malefyt, R., Coffman, R. L., and O'Garra, A.: Interleukin 10 and the interleukin 10 receptor. Ann Rev Immunol 19: 6833-765, 2001.

271. Alexander, W. S., Starr, R., Fenner, J. E., et al.: SOCS1 is a critical inhibitor of interferon-γ signaling and prevents the potentially fatal neonatal actions of this cytokine. Cell 98: 597-608, 1999.

272. Stoiber, D., Kovatik, P., Cohney, S., Johnston, J. A., Steinlein, P., and Decker, T.: Lipopolysaccharide induces in macrophages the synthesis of the suppressor of cytokine signaling 3 and suppresses signal transduction in response to the activating factor IFN-γ. J Immunol 163: 2640-2647, 1999.

273. O'Keefe, G. M., Nguyen, V. T., Tang, L. P., and Benveniste, E. N.: IFN-γ regulation of class II transactivator promoter IV in macrophages and microglia: involvement of the SOCS-1 protein. J Immunol 166: 2260-2269, 2001.

274. Dickensheets, H. L., Venkataraman, C., Schindler, U., and Donnelly, R. P.: Interferons inhibit activation of STAT6 by interleukin4 in human monocytes by inducing SOCS-1 gene expression. Proc Nat Acad Sci USA 96: 10800-10805, 1999.

275. Crespo, A., Filla, M. B., and Murphy, W. J.: Low responsiveness to IFN-γ after pretreatment of mouse macrophages with LPS develops via diverse regulatory pathways. Eur J Immunol 32: 710-719, 2002.

276. Drake, W. P., Pei, Z., Pride, D. T., Collins, R. D., Cover, T. L., and Blaser, M. J.: Molecular analysis of sarcoidosis tissues for Mycobacterium species DNA. Emerg Infect Dis 11: 1334-1341, 2002.

277. Dijkstra, C. D., Dopp, E. A., Joling, P., and Kraal, G.: The heterogeneity of mononuclear phagocytes in lymphoid organs: distinct macrophage subpopulations in rat recognized by monoclonal antibodies ED1, ED2 and ED3. Adv Exp Med Biol 186: 409-19, 1985.

278. Hubeau, C., Lorenzato, M., Couetil, J. P., et al.: Quantitative analysis of inflammatory cells infiltrating the cystic fibrosis airway mucosa. Clin Exp Immunol 124: 69-76, 2001.

279. Umemoto, E. Y., Brokaw, J. J., Dupuis, M., and McDonald, D. M.: Rapid changes in shape and number of MHC class II expressing cells in rat airways after Mycobacterium pulmonis infection. Cell Imnmunol 220: 107-115, 2002.

280. Nicod, L. P., and Isler, P.: Alveolar macrophages in sarcoidosis coexpress high levels of CD86, CD40, and CD30L. Am J Resp Cell Mol Biol 17: 91-96, 1997.

281. Ferrari-Lacraz, S., Nicod, L. P., Chicheportiche, R., Welgus, H. G., and Dayer, J. M.: Human lung tissue macrophages but not alveolar macrophages express matrix metalloproteinases after direct contact with activated T lymphocytes. Am J Resp Cell Mol Biol 24: 442-451, 2001.

282. Moore, T. M., Shirah, W. B., Khimenko, P. L., et al.: Involvement of CD40-CD40L signaling in postischemic lung injury. Am J Physiol Lung Cell Mol Physiol 283: L1255-L1262, 2002.

283. Hille, R.: Molybdenum and tungsten in biology. Trends in Biochem Sci. 27:360-367, 2002.

284. Vorbach, C., Scriven, A., and Capecchi, M. R.: The housekeeping gene Xanthine oxidoreductase is necessary for milk fat droplet enveloping and secretion: gene sharing in the lactating mammary gland. Genes & Dev 16: 3223-3235, 2002.

285. Agostini, C., Perin, A., and Semenzato, G.: Cell apoptosis and granulornatous lung diseases. Curr Opin Pulm Med 4: 261-266, 1998.

286. Kunitake, R., Kuwano, K., Miyazaki, H., Hafimoto, N., Nomoto, Y., and Hara, N: Apoptosis in the course of granulomatous inflammation in pulmonary sarcoidosis. Eur Resp J 13: 1329-1337, 1999.

287. Dai, H., Guzman, J., and Costabel, U.: Increased expression of apoptosis signaling receptors by alveolar macrophages in sarcoidosis. Eur Respir J 13: 1451-1454, 1999.

288. Shikuwa, C., Kadota, J., Mukae, H., Iwashita, T., Kaida, H., Ishii, H., Ishimatsu, Y., and Kohno, S.: High concentrations of soluble Fas ligand in bronchoalveolar lavage fluid of patients with pulmonary sarcoidosis. Respiration 69: 242-246, 2002.

289. Stridh, H., Planck, A., Giglotti, D., Eklund, A., and Grunewald, J.: Apoptosis resistant bronchoalveolar lavage fluid (BAL) lymphocytes in sarcoidosis. Thorax 57: 897-901, 2002.

290. Xaus, J., Besalduch, N., Comalada, M., Marcoval, J., Pujol, R., ana, J., and Celada, A.: High expression of p21wafl in sarcoid granuloma: a putative role for long-lasting inflammation. J Leuko Biol 74: 295-301, 2003.

291. Baldus, S., Eiserich, J. P., Brennan, M. L., Jackson, R. M., Alexander, C. B., and Freeman, B. A.: Spatial mapping of pulmonary and vascular nitrotyrosine reveals the pivotal role of myeloperoxidase as a catalyst for tyrosine nitration in inflammatory diseases. Free Rad Biol Med 33: 1010-1019, 2002.

292. Haddad, I. Y., Pataki, G., Hu, P., Galliani, C., Beckman, J. S., Matalon, S.: Quantitation of nitrotyrosine in lung sections of patients and animals with acute lung injury. J Clin Invest 94: 2407-2413, 1994.

293. Martin, T. R.: Lung cytokines and ARDS. Chest 116: 2-8, 1999.

294. Hacker, C., Kirsch, R. D., Ju, X. S., et al.: Transcriptional profiling identifies Id2 function in dendritic cell development. Nature Immunol 4:380-386, 2003.

295. Xu, M., Nie, L., Kim, S. H., and Sun, X. H.: STAT5 induced Id-1 transcription involves recruitment of HDAC1and deacetylation of C/EBP-β. The EMBO J. 22: 893-904, 2003.

We claim:

1. A method of modulating inflammatory reactions involving leukocytes and leukocyte precursor cells in a subject comprising contacting a subject in need of said modulating with an amount of a xanthine oxidoreductase (XOR) inhibitor effective to modulate said inflammatory reaction mediated by leukocytes and leukocyte precursor cells in said subject.

2. The method of claim 1, wherein said leukocytes and leukocyte precursor cells are selected from the group consisting of mononuclear phagocytes, neutrophils and eosinophils.

3. The method of claim 1 wherein said inflammatory reaction is a disease selected from the group consisting of chronic heart failure, cardiomyopathy, diabetes, pancreatic inflammation, liver inflammation, Crohn's disease, uveitis, acute lung injury, COPD, sarcoidosis, granulomatous lung inflammation (GLI), acute lymphoblastic leukemia (ALL), ischemia reperfusion injury, hemorrhagic shock and renal transplant rejection.

4. The method of claim 3 wherein said inflammatory disease is granulomatous lung inflammation.

5. The method of claim 3 wherein said inflammatory disease is acute or chronic lung injury.

6. The method of claim 1 wherein said oxidoreductase inhibitor is selected from the group consisting of allopurinol, oxypurinol, tungsten, amflutizole, (−)BOF-4272 ([(−)-8-(3-methoxy-4-phenylsulfinylphenyl)pyrazolo(1, 5-alpha)-1, 3, 5-triazine-4-monohydrate]), diphenyleneiodonium dichloride, glutathione, glutathione precursors and dimethylthiourea (DMTU).

7. A method of modulating XOR activity in leukocytes and leukocyte precursor cells comprising contacting said leukocytes and leukocyte precursor cells with an agent which modulates the expression, synthesis, degradation, secretion, release, half-life, conversion or catalysis of XOR in leukocytes and leukocytes precursor cells thereby modulating XOR activity.

8. The method of claim 7, wherein said leukocytes and leukocyte precursor cells are selected from the group consisting of mononuclear phagocytes, neutrophils and eosinophils.

9. The method of claim 7, wherein said contacting occurs in vitro.

10. The method of claim 7, wherein said contacting occurs in vivo.

11. The method of claim 7, wherein said leukocytes and leuckocyte precursor cells are involved in inflammatory reactions.

12. The method of claim 11, wherein said inflammatory reaction is an inflammatory disease selected from the group consisting of chronic heart failure, cardiomyopathy, diabetes, pancreatic inflammation, liver inflammation, Crohn's disease, uveitis, acute lung injury, COPD, sarcoidosis, granulomatous lung inflammation (GLI), acute lymphoblastic leukemia (ALL), ischemia reperfusion injury, hemorrhagic shock and renal transplant rejection.

13. The method of claim 12, wherein said inflammatory disease is granulomatous lung inflammation.

14. The method of claim 12, wherein said inflammatory disease is acute lung injury.

15. The method of claim 7, wherein said agent is selected from the group consisting of allopurinol, oxypurinol, tungsten, amflutizole, (−)BOF-4272, diphenyleneiodonium dichloride, glutathione, glutathione precursors and dimethylthiourea (DMTU).

16. A method of modulating inflammatory reactions involving leukocytes and leukocyte precursor cells in a subject comprising contacting said subject in need of said modulating with an amount of an agent which is effective to modulate the expression, synthesis, degradation, secretion, release, half-life, conversion or catalysis of XOR in leukocytes and leukocyte precursor cells thereby modulating said inflammatory reactions.

17. The method of claim 16, wherein said leukocytes and leukocyte precursor cells are selected from the group consisting of mononuclear phagocytes, neutrophils and eosinophils.

18. The method of claim 16, wherein said inflammatory reaction is an inflammatory disease selected from the group consisting of chronic heart failure, cardiomyopathy, diabetes, pancreatic inflammation, liver inflammation, Crohn's disease, uveitis, acute lung injury, COPD, sarcoidosis, granulomatous lung inflammation (GLI), acute lymphoblastic leukemia (ALL), ischemia reperfusion injury, hemorrhagic shock and renal transplant rejection.

19. The method of claim 18, wherein said inflammatory disease is granulomatous lung inflammation.

20. The method of claim 18, wherein said inflammatory disease is acute lung injury.

21. The method of claim 16, wherein said agent is selected from the group consisting of allopurinol, oxypurinol, tungsten, amflufizole, (−)BOF-4272, diphenyleneiodonium dichloride, glutathione, glutathione precursors and dimethylthiourea (DMTU).

22. A method of modulating cytokine-induced inflammation in a subject comprising
a) removing leukocytes and leukocyte precursor cells from said subject to form a population of leukocytes and leukocyte precursor cells;
b) contacting said population of leukocytes and leukocyte precursor cells formed in part a) with an effective amount of one or more agents to obtain a treated cell population, wherein said one or more agents are effective to modulate the expression, synthesis, degradation, secretion, release, half-life, conversion or catalysis of XOR in leukocytes and leukocyte precursor cells; and,
c) administering said treated cell population of part b) to said subject;
wherein said administered cell population modulates said cytokine-induced inflammation in said subject.

23. The method of claim 22, wherein said leukocytes and leukocyte precursor cells are selected from the group consisting of mononuclear phagocytes, neutrophils and eosinophils.

24. The method of claim 22, wherein said agent is an inhibitor.

25. The method of claim 24, wherein said inhibitor is selected from the group consisting of allopurinol, oxypurinol, tungsten, amflutizole, (−)BOF-4272, diphenyleneiodonium dichloride, glutathione, glutathione precursors and dimethylthiourea (DMTU).
